SEQUENCE LISTING (21 sequences)

SEQ ID NO: 1 - DNA, 20 bp, Artificial Sequence
  Synthetic
gagagtcctggtctttgtca

SEQ ID NO: 2 - DNA, 20 bp, Artificial Sequence
  Synthetic
acagtgcctaggaagggtat

SEQ ID NO: 3 - DNA, 20 bp, Artificial Sequence
  Synthetic
acagtgcctaggaagggtat

SEQ ID NO: 4 - DNA, 20 bp, Artificial Sequence
  Synthetic
tacttggtttctgcatcctt

SEQ ID NO: 5 - DNA, 20 bp, Artificial Sequence
  Synthetic
tcactaaagaaaccgttcgt

SEQ ID NO: 6 - DNA, 19 bp, Artificial Sequence
  Synthetic
gagcagaggtgggagttag

SEQ ID NO: 7 - DNA, 19 bp, Artificial Sequence
  Synthetic
gagcagaggtgggagttag

SEQ ID NO: 8 - DNA, 126 bp, Artificial Sequence
  Synthetic
  misc_feature (84)..(102): n is a, c, g, or t
aaaaaaagcaccgactcggtgccactttttcaagttgataacggactagccttattttaacttgctatttctagctctaaaacnnnnnnnnnnnnnnnnnnnccggtgtttcgtcctttccacaag

SEQ ID NO: 9 - DNA, 23 bp, Artificial Sequence
  Synthetic
gagggcctatttcccatgattcc

SEQ ID NO: 10 - DNA, 25 bp, Artificial Sequence
  Synthetic
  misc_feature (6)..(25): n is a, c, g, or t
caccgnnnnnnnnnnnnnnnnnnnn

SEQ ID NO: 11 - DNA, 25 bp, Artificial Sequence
  Synthetic
  misc_feature (5)..(24): n is a, c, g, or t
aaacnnnnnnnnnnnnnnnnnnnnc

SEQ ID NO: 12 - DNA, 22 bp, Artificial Sequence
  Synthetic
agagctggaacgttacctccag

SEQ ID NO: 13 - DNA, 22 bp, Artificial Sequence
  Synthetic
agagctggaacgttacctccag

SEQ ID NO: 14 - DNA, 17 bp, Artificial Sequence
  Synthetic
tccctccagggctggtg

SEQ ID NO: 15 - DNA, 23 bp, Artificial Sequence
  Synthetic
ttgagttggcagtctttgcttgg

SEQ ID NO: 16 - DNA, 20 bp, Artificial Sequence
  Synthetic
ccatgactcggttttgctct

SEQ ID NO: 17 - DNA, 20 bp, Artificial Sequence
  Synthetic
ggtgaactgggcttacagga

SEQ ID NO: 18 - DNA, 17 bp, Artificial Sequence
  Synthetic
tagagctaattgtggct

SEQ ID NO: 19 - DNA, 19 bp, Artificial Sequence
  Synthetic
gatttgctcaatttgaaag

SEQ ID NO: 20 - DNA, 20 bp, Artificial Sequence
  Synthetic
actgaagatgagatcactgt

SEQ ID NO: 21 - DNA, 14935 bp, Artificial Sequence
  Synthetic
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagccccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaaataacttcgtatagcatacattatacgaagttatgataagagacggtggtggcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccactgcatgacgtctccacaattaattaagggtgcagcggcctccgcgccgggttttggcgcctcccgcgggcgcccccctcctcacggcgagcgctgccacgtcagacgaagggcgcaggagcgttcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagttgcgggctgctgggctggccggggctttcgtggccgccgggccgctcggtgggacggaagcgtgtggagagaccgccaagggctgtagtctgggtccgcgagcaaggttgccctgaactgggggttggggggagcgcacaaaatggcggctgttcccgagtcttgaatggaagacgcttgtaaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcgggtttgtcgtctggttgcgggggcggcagttatgcggtgccgttgggcagtgcacccgtacctttgggagcgcgcgcctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagcttgggctgcaggtcgactctagagccaccatggactacaaagaccatgacggtgattataaagatcatgacatcgattacaaggatgacgatgacaagatggcccccaagaagaagaggaaggtgggccgcggaatggacaagaagtactccattgggctcgccatcggcacaaacagcgtcggctgggccgtcattacggacgagtacaaggtgccgagcaaaaaattcaaagttctgggcaataccgatcgccacagcataaagaagaacctcattggcgccctcctgttcgactccggggaaaccgccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatgccatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtggctagccgcgccgacgcgctggacgatttcgatctcgacatgctgggttctgatgccctcgatgactttgacctggatatgttgggaagcgacgcattggatgactttgattggacatgctcggctccgatgctctggacgatttcgatctcgatatgttaatcgctagcgagggcagaggaagtcttctaacatgcggtgacgtggaggagaatcccggccctggtaccgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagaccggttgataatagataacttcgtatagcatacattatacgaagttatgaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

(19) United States
(12) Patent Application Publication
(10) Pub. No.: US 2022/0125851 A1
(43) Pub. Date: Apr. 28, 2022

(54) MATERIALS AND METHODS FOR BONE MARROW TRANSPLANTATION

(71) Applicant: UNIVERSITY OF MIAMI, Miami, FL (US)

(72) Inventors: Maria E. Figueroa, Miami, FL (US); Alejandro Roisman, Miami, FL (US)

(21) Appl. No.: 17/508,779

(22) Filed: Oct. 22, 2021

Related U.S. Application Data
(60) Provisional application No. 63/198,517, filed on Oct. 23, 2020.

(51) Int. Cl. A61K 35/28; C12N 5/0789; C12N 15/11; C12N 9/22

(52) U.S. Cl. CPC A61K 35/28; C12N 5/0647; A61K 2035/124; C12N 9/22; C12N 15/111

(57) ABSTRACT

The disclosure provides a method of preparing HSPC for bone marrow transplantation, the method comprising (a) obtaining donor hematopoietic stem and progenitor cells and (b) upregulating expression of Kruppel-like factor 6.

### CROSS REFERENCE TO RELATED APPLICATION

This application claims priority to U.S. Provisional Patent Application No. 63/198,517, filed Oct. 23, 2020, which is hereby incorporated by reference in its entirety.

### FIELD OF DISCLOSURE

The disclosure provides materials and methods for enhancing donor material for bone marrow transplantation.

### INCORPORATION BY REFERENCE OF MATERIALS SUBMITTED ELECTRONICALLY

The Sequence Listing, which is a part of the present disclosure, is submitted concurrently with the specification as a text file. The name of the text file containing the Sequence Listing is “56047A_Seqlisting.txt”, which was created on Jan. 7, 2022 and is 23,795 bytes in size. The subject matter of the Sequence Listing is incorporated herein in its entirety by reference.

### BACKGROUND

The hematopoietic system comprises a hierarchy of cells that evolve from a stem cell condition to the differentiated state that will give rise to all blood linages. Hence, precise and accurate hematopoietic stem cell (HSC) function is fundamental in order to maintain the normal production of all blood linages through an organismal lifespan. However, aging remains as the most predominant influence for hematological disorders and as an organism ages, generally there is a continuous and progressive inability to maintain homeostasis which results in gradual impairment of HSC function. As such, aged HSC are characterized by impairment in self-renewal and homing capabilities, decreased lymphoid potential eliciting a myeloid differentiation bias as well as an increased risk of malignant transformation. (Lopez-Otin et al, Cell, 153 (6), P1194-1217, 2013). These effects of aging impact the suitability of hematopoietic stem and progenitor cells (HSP) for bone marrow transplantation. Indeed, in many cases, physicians request donors aged 45 years or less because donor material from younger subjects are associated with better outcomes.

There remains a need in the art for materials and methods of treating donor material to improve properties associated with successful bone marrow transplantation.

### SUMMARY

The disclosure provides method of preparing HSPC for bone marrow transplantation, the method comprising (a) obtaining donor hematopoietic stem and progenitor cells and (b) upregulating expression of Kruppel-like factor 6 (KLF6). In various aspects, method further comprises (c) administering the HSPC of step (b) to a subject in need thereof.

### BRIEF DESCRIPTION OF THE DRAWINGS

This patent or application file contains at least one drawing executed in color. Copies of this patent or patent application publication with color drawing(s) will be provided by the United States Patent and Trademark Office upon request and payment of the necessary fee.

FIGS. 1A-1G: Loss of KLF6 in HSC Recapitulates Physiological Hematopoietic Aging In Vivo. FIG. 1A illustrates an experimental design to determine the hematopoietic reconstitution upon KLF6 KO. FIG. 1B illustrates the results of kinetics examination of peripheral blood engraftment in KLF6 KO and NTC xenotransplanted mice. FIG. 1C illustrates peripheral blood linage reconstitution analysis in peripheral blood between KLF6 KO and NTC specimens. FIG. 1D is a graph illustrating peripheral blood myeloid to lymphoid ratio weeks 4 and 16 post-injection. FIG. 1E illustrates bone marrow engraftment examination in KLF6 KO and NTC xenotransplanted mice. FIG. 1F illustrates HSC compartment comparison in specimens injected with KLF6 KO and control HSPCs. FIG. 1G illustrates progenitor comparison output analysis in bone marrow at the endpoint of the experimental design.

FIGS. 2A-2I: KLF6 is regulated by an enhancer region found 20 kb upstream of its promoter in HSPCs. FIG. 2A is a comparison of KLF6 expression in KLF6 disrupted cells and −20 Kb enhancer, −25 Kb enhancer and −66 Kb enhancer targeted cells. FIG. 2B illustrates flow cytometry (FC) analysis of CD33 expression in in KLF6 disrupted cells, −20 Kb enhancer, −25 Kb enhancer and −66 Kb enhancer targeted HSPCs. FIG. 2C is FC analysis of CD11b expression in in KLF6 disrupted cells and −20 Kb enhancer, −25 Kb enhancer and −66 Kb enhancer targeted cells. FIG. 2D is FC analysis of CD71 expression in KLF6 disrupted cells and −20 Kb enhancer, −25 Kb enhancer and −66 Kb enhancer targeted cells. FIG. 2E is a Venn diagram displaying the transcriptional overlap between KLF6 KO and −20 Kb enhancer targeted HSPC. FIG. 2F is a bar plot representation of normalized enrichment score (NES) for select genes enrichment pathways that are upregulated in KLF6 KO and −20 Kb enhancer targeted HSPC. FIG. 2G is a bar plot representation of normalized enrichment score (NES) for select genes enrichment pathways that are downregulated in KLF6 KO and −20 Kb enhancer targeted HSPC. FIG. 2H is bar graphs and illustrations providing DNA damage quantification of γH2AX in KLF6 disrupted cells and −20 Kb enhancer, −25 Kb enhancer and −66 Kb enhancer targeted cells. FIG. 2I is a bar graph and illustration showing representative apoptosis quantification in KLF6 disrupted cells and −20 Kb enhancer, −25 Kb enhancer and −66 Kb enhancer targeted cells.

FIGS. 3A-3F: KLF6 Enhancer Disruption Promotes Aging Like Features In Vivo. FIG. 3A relates to kinetics examination of peripheral blood engraftment in −20 Kb enhancer targeted and NTC HSPCs xenotransplanted mice. FIG. 3B relates to peripheral blood linage reconstitution analysis in peripheral blood between −20 Kb enhancer targeted and NTC specimens. FIG. 3C relates to peripheral blood myeloid to lymphoid dynamics weeks 4 and 16 post-injection. FIG. 3D relates to bone marrow engraftment examination measurement in −20 Kb enhancer targeted and NTC specimens. FIG. 3E relates to HSC compartment comparison in specimens injected with −20 Kb enhancer targeted and NTC HSPCs. FIG. 3F relates to progenitor comparison output analysis in bone marrow at the endpoint of the experimental design in specimens injected with −20 Kb enhancer targeted and NTC.

FIGS. 4A-4D: KLF6 induction in aged HSCs improves phenotypic changes associated with HSC aging. FIG. 4A illustrates an experimental design to determine the impact of activating KLF6 in aged normal donors in comparison with young unmanipulated HSPCs, young NTC HSPC, aged unmanipulated HSPCs and aged NTC HSPCs. FIG. 4B is representative flow cytometry histograms of KLF6 expression in young and aged controls and KLF6 activated HSPCs. FIG. 4C is flow cytometry (FC) analysis of CD33 expression in KLF6 activated aged donors in comparison with young unmanipulated HSPCs, young NTC HSPC, aged unmanipulated HSPCs and aged NTC HSPCs. FIG. 4D is FC analysis of CD11b expression in KLF6 activated aged donors in comparison with young unmanipulated HSPCs, young NTC HSPC, aged unmanipulated HSPCs and aged NTC HSPCs.

FIG. 5: Illustration of experimental strategy.

FIG. 6: Illustration of age-related changes in histone modifications that target regulatory elements.

FIG. 7: Illustrations relating to enhancer deregulation as a feature of aged HSCe.

FIG. 8: Illustrations relating to enhancer deregulation as a feature of aged HSCe which targets immune and cancer pathways.

FIG. 9: Illustrations relating to transcription factors that are enriched at deregulated enhancers.

FIG. 10: Illustrations demonstrating that aged HSCe display down-regulation of epigenetic modifiers and hematopoietic transcription factors.

FIG. 11: Illustrations demonstrating epigenetic changes at enhancers and promoters that mediate age-related expression changes.

FIG. 12: Illustrations demonstrating that age-related epigenetic changes are the result of HSCe reprogramming.

FIG. 13: Illustrations demonstrating that KLF6 is downregulated with aging in HSCe.

FIG. 14: Illustrations relating to experimental strategy.

FIG. 15: Illustrations relating to KLF6 locus editing using CRISPR-Cas9 in human CD34+ cells.

FIG. 16: Illustrations demonstrating that loss of KLF6 leads to increased colony formation and differentiation block.

FIG. 17: Illustrations demonstrating that loss of KLF6 recapitulates aging HSCe and AML profiles.

FIG. 18: Illustrations demonstrating that loss of KLF6 leads to epigenetic reprogramming.

FIG. 19: Illustrations demonstrating that loss of KLF6 leads to aged-like hematopoiesis differentiation in vivo.

FIG. 20: Illustration relating to KLF6 enhancer.

FIG. 21: Illustration demonstrating that deletion of KLF6 enhancer recapitulates differentiation and CFU phenotypes.

FIG. 22: Illustration demonstrating that deletion of KLF6 enhancer recapitulates profile of KLF6 knock out.

FIG. 23: Illustration demonstrating that deletion of KLF6 enhancer leads to aged-like hematopoiesis in vivo.

FIG. 24: Illustrations relating to experimental strategy.

FIG. 25: Illustration demonstrating that re-expression of KLF6 reverses differentiation defect.

FIGS. 26A-26D: Profound epigenetic reprogramming with aging in primary human HSCe. (FIG. 26A) Heatmap representation of enhancer regions with age-related reduction in H3K27ac. (FIG. 26B) Bubble plot representation of pathways associated with genes annotated to these enhancers (FDR <0.05). (FIG. 26C) Heatmap representation of bivalent promoters with age-related reduction in H3K4me3. (FIG. 26D) Bubble plot representation of pathways associated with bivalent promoters (FDR <0.05).

FIGS. 27A-27C: Age-related changes in 5hmC are enriched for GATA and KLF motifs. Heatmaps and density plots showing differentially hydroxymethylated regions (DHMR) with gains in 5hmC in aged HSCe. Distribution of 5hmC peaks in young compared to regions with gained peaks in aging. (FIG. 27C) DNA motifs enriched in age-related DHMRs.

FIGS. 28A-28B: Age-related epigenetic reprogramming results in downregulation of KLF6 (FIG. 28A) Volcano-plot of genes that are differentially expressed in aged HSCe compared to young. (FIG. 28B) UCSC tracks of pooled replicates for young and aged HSCe at the KLF6 locus highlighting 3 of 8 putative enhancers identified with age-associated decrease in H3K27ac (black boxes).

FIGS. 29A-29D: KLF6 KO results in increased CFU and strong myeloid and erythroid differentiation block. (FIG. 29A) KLF6 editing in CD34+ validated by T7E1 assay (left), qRT-PCR (middle) and flow cytometry quantification of KLF6 protein levels (right). (FIG. 29B) CFU assays in which KLF6 was knocked out in healthy CD34+ cells (n=5). (FIG. 29C) Representative flow cytometry histograms for 2 donors and contour plots from 1 donor for CD34+ cells transfected with sgCTRL or sgKLF6 and cultured in myeloid promoting conditions for 7 days. (FIG. 29D) Dot plot representation of the percentage of CD34+ cells (top) CD34−CD11b+ (middle) and CD235a− CD71+ (bottom) cells assessed by flow cytometry after culturing in myeloid or erythroid conditions (n=5) (*p<0.05; **p<0.01).

FIGS. 30A-30C: KLF6 knock-out results in expression changes similar to those observed in aging and in cancer. (FIG. 30A) Heatmap of genes differentially expressed upon KLF6 KO (p adj<0.05, fold-change>1.5). (FIG. 30B) Enrichment by GSEA of the HSCe aging signature upon KLF6 KO. (FIG. 30C) Top 5 up and downregulated gene sets enriched by GSEA in KLF6 deficient CD34+ cells show string enrichment for leukemia-associated gene sets.

FIGS. 31A-31D: Deletion of a KLF6 enhancer recapitulates the gene knock-out phenotype. (FIG. 31A) UCSC tracks showing the KLF6 locus and the location of three putative enhancers at −20, −25 and −66 kb with reduction of the H3K27ac signal (purple tracks) in aged HSCe (FIG. 31B) Despite genomic effective deletion of all three putative enhancers, only the −20 kb deletion resulted in downregulation of the mRNA (by qPCR) and protein (by FACS) (FIG. 31C) Contour plot of a representative experiment out of 3 replicates, showing block in myeloid and erythroid differentiation in liquid culture after 7 days with deletion of the −20 enhancer (FIG. 31D) CFU analysis demonstrated that deletion of the −20 kb, but not the −25 or the −66 kb regions recapitulates the enhanced colony-formation phenotype observed with deletion of the gene itself.

FIGS. 32A-32B: Re-expression of KLF6 in aged CD34+ via CRISPRa improves the myeloid differentiation phenotype of these cells. (FIG. 32A) qRT-PCR quantification of KLF6 levels 7 days post dCas9-VP64 activation of KLF6. (FIG. 32B) Flow cytometry evaluation of the myeloid differentiation markers CD33 (top) and CD11b (bottom) after 7 days under myeloid differentiation culture conditions. Unmanipulated (No CRISPRa) and NTC aged cells differentiated more efficiently into myeloid cells than their younger counterparts, but this phenotype was abrogated when KLF6 was re-expressed using CRISPRa and KLF6-targeting sgRNA in aged cells, while KLF6 CRISPRa had no effect in young cells. (Representative experiment out of 3 biological replicates).

FIG. 33: KLF6 KO results in myeloid bias and HSC expansion in NSGS mice. Peripheral blood engraftment analysis of percentage of human CD45+ cells, CD19+ cells and CD33+ cells in our study showed no significant differences in engraftment between non-targeting control (NTC) and KLF6 KO groups, but KLF6 KO resulted in a significant myeloid bias (*p<0.01). In addition, analysis of BM composition at 14 weeks post-transplant revealed that KLF6 KO resulted in a statistically significant increase in HSC, while GMP and MEP showed a trend towards an increase (data not shown).

FIGS. 34A-34D: Experimental design and validation of KLF6 expression in primary human CD34+ and leukemia cell lines. (FIG. 34A) Experimental design for studying the effects of KLF6 loss in HSPC in-vitro. (FIG. 34B) Bar plot representation of mRNA quantification in young healthy CD34+ cells targeted for NTC or KLF6. (FIG. 34C) Validation of KLF6 expression protein levels in leukemia cell lines by western blot (top left). Correlation of flow cytometry and western blot expression intensity in leukemia cell lines (bottom left). Densitometry was performed using the LI-COR Image Studio software. Intensity of KLF6 was normalized to that of a loading control, histone H3. Pearson coefficient was used to calculate the correlation between the flow cytometry and western blot expression data. Representative histograms displaying KLF6 protein levels in young healthy human CD34+ cells targeted for NTC of KLF6 (middle). Quantification of KLF6 positive cells in young healthy human CD34+KLF6 KO or NTC cells (right). (FIG. 34D) Representative FACS gating strategy plots for evaluation of peripheral blood lineage reconstitution (left) and for assessing bone marrow hematopoietic reconstitution (right).

FIGS. 35A-35J: KLF6 deficiency reprograms epigenetic states and recapitulates the myeloid bias and HSPC expansion observed during aging in vivo. FIG. 35A) Experimental design for studying the effects of KLF6 depletion in HSPC in NSGS xenotransplants. (FIG. 35B) Fluorescence-activated cell sorting (FACS) plot of human CD19 and CD33 expression in peripheral blood 6 weeks after xenotransplant with KLF6 KO or NTC HSPCs and quantification of these markers throughout engraftment. (FIG. 35C-FIG. 35D) Representative FACS plot of the HSC (FIG. 35C) and progenitor (FIG. 35D) compartments in bone marrow of NSGS mice 16 weeks after transplantation with KLF6 KO HSPCs and quantification of these populations. (FIG. 35E) Heatmap representation of regions with either loss or gain (FDR <0.05) of H3K27ac or H3K4me3 signal in young NTC HSPC compared to young KLF6 KO HSPC. The log 2(IP/Input) signal is plotted for each replicate, centered on the differential peak +/−5 kb. Each column is representative of an individual replicate. (FIG. 35F) Bubble plot representation of select gene ontology biological processes enriched for H3K27ac after KLF6 depletion in HSPCs. The size of each bubble corresponds to the number of genes within the cluster that are within the given gene set. Highly significant (FDR <0.05) categories are colored in purple. (FIG. 35G) UCSC genome browser track examples of genes with reduced H3K27ac signal after KLF6 KO in HSPCs. Tracks are normalized to input and read number. (FIG. 35H) Example of transcription factor binding DNA motifs enriched in regions that display loss of H3K27ac after KLF6 KO. (FIG. 35I) UCSC genome browser track examples of genes with increased H3K27ac signal in KLF6 KO HSPCs. (FIG. 35J) Example of transcription factor binding DNA motifs enriched in regions that display gains of H3K27ac after loss of KLF6. p-values p-values from linear mixed-effects model or paired t-test, calculated with R with the nlme package or Prism, p>0.05=ns, p≤0.05=*, p≤0.01=**, p≤0.001=***, p≤0.0001=****.

FIGS. 36A-36K: Effects of KLF6 depletion in hematopoietic reconstitution and epigenomic distribution. (FIG. 36A) Representative flow cytometry plot showing peripheral blood contribution of human CD45+ in KLF6 disrupted and NTCs HSPCs at week 6 of reconstitution and summary of reconstitution throughout the experiment (n=18 per group). Significance was assessed using a linear mixed-effects model with the parameter examined. (FIG. 36B) Representative flow cytometry plot assessing peripheral blood myeloid to lymphoid ratio observed in KLF6 disrupted and NTCs HSPCs at week 8 post-transplant and summary throughout the experiment. Significance was assessed using a linear mixed-effects model with the parameter examined. Representative flow cytometry plots and bar plot summary showing MPP (FIG. 36C), CMP (FIG. 36D), MEP (FIG. 36E), GMP (FIG. 36F) and lymphoid (FIG. 36G) fractions in NSGS mice transplanted with KLF6 KO or NTC HSCPs. Significance was assessed using a two-tailed unpaired t-test (n=8, 7 respectively). (FIG. 36H) Heatmap representation of regions with reduced (FDR <0.05) H3K4me3 signal in young NTC HSPC compared to young KLF6 KO HSPC. The log 2(IP/Input) signal is plotted for each replicate, centered on the differential peak+/−5 kb. Each column is representative of an individual replicate. (FIG. 36I) Bar plots illustrating the percentage of peaks that are gained or lost in KLF6 KO HSPCs compared to NTC HSPCs. (FIG. 36J-FIG. 36K) Overlap between the significant differential peaks that lose H3K27ac (FIG. 36J) or H3K4me3 (FIG. 36K) with normal aging in HSCe and with KLF6 depletion.

FIGS. 37A-37E: KLF6 enhancer E1 regulates KLF6 activity in vitro in HSPCs. (FIG. 37A) UCSC ChIP-seq tracks of H3K4mel, H3K4me3, and H3K27ac at the KLF6 promoter region in young and aged human HSCe. (FIG. 37B) T7E1 assays performed in E1, E2 or E3 disrupted HSPCs. PCR amplicons were either treated (+) or untreated (−) with T7E1. Arrowheads indicate the bands with expected size based on the Cas9 cleavage site. The numbers below the gel image represent the cleavage efficiency determined by densitometric analysis. (FIG. 37C) Colony-forming unit assays targeting NTC, KLF6, E1, E2 or E3 region in young healthy human CD34+ cells. Colony numbers per 500 CD34+ cells plated are plotted for total colony number, granulocyte-macrophage (GM), granulocyte-erythrocyte-macrophage-megakaryocyte (GEMM) and burst-forming unit erythroid (BFU-E). Colony numbers for each biological replicate (n=3) are normalized to the total colony number for that replicate. (FIG. 37D-FIG. 37E) Representative flow cytometry plots of NTC, KLF6, E1, E2 or E3 of targeted young healthy human CD34+ cells cultured in myeloid (FIG. 37D) or erythroid (FIG. 37E) promoting conditions. Bar plot (right) representing the differentiation potential of young healthy human CD34+ cells at each targeted region.

FIGS. 38A-38J: Identification of a transcriptionally regulatory region responsible for KLF6 function in HSPCs. (FIG. 38A) H3K4mel and H3K27ac UCSC genome browser tracks of KLF6 and putative enhancers E1, E2 and E3 in young and aged HSCe. Tracks are of pooled replicates for HSCe of young and age healthy donors, normalized to reads per million and to the corresponding Input. (FIG. 38B) Representative histograms displaying KLF6 protein levels in young healthy human CD34+ cells targeted for NTC, KLF6, E1, E2 or E3, as measured by flow cytometry 48 hr after CRISPR-Cas9 transduction (left) and barplot of quantification (n=3) (right). (FIG. 38C) Bar plot representation of mRNA quantification in young healthy CD34+ cells targeted for NTC, KLF6 and E1, 48 hr after CRISPR-Cas9 transduction. (FIG. 38D) Bar plot of colony-forming unit assays performed with young healthy human CD34+ cells targeted with NTC, KLF6, E1, E2 or E3. Colony numbers per 500 CD34+ cells plated are plotted for total colony number, granulocyte-macrophage (GM), granulocyte-erythrocyte-macrophage-megakaryocyte (GEMM) and burst-forming unit erythroid (BFU-E). Colony numbers for each biological replicate (n=3) are normalized to the total colony number for that replicate. (FIG. 38E-FIG. 38F) Representative flow cytometry plots of NTC, KLF6 and E1 targeted young healthy human CD34+ cells cultured in myeloid (FIG. 38E) or erythroid (FIG. 38F) promoting conditions. Bar plot (right) summarizing the differentiation potential of young healthy human CD34+ cells at each targeted region (n=3). (FIG. 38G) Venn-diagram that shows the overlap of genes that are differentially expressed (FDR <0.05 and absolute fold-change >2) with loss of KLF6 or disruption of E1 in young healthy human CD34+. (FIG. 38H) Bubble plot representation of the normalized enrichment score (NES) for select gene set enrichment pathways that are up or downregulated in KLF6 KO or E1 KO young healthy human CD34+ cells (FDR<0.1). (FIG. 38I) Dot plot of FLI1, ERG, and RUNX1 gene expression in young and aged HSCe (n=10 per group) as previously assayed by RNA-seq. Adjusted p-values and fold-change (FC) were determined by DESeq2. (FIG. 38J) Bar plots of KLF6 mRNA expression in HUDEP-2 cells with siRNA mediated knock-down of FLI1, RUNX1 or ERG in HUDEP-2 cells and mRNA expression of the siRNA targeting. p-values from t-test, calculated with Prism, p>0.05=ns, p≤0.05=*, p≤0.01=**, p≤0.001=***, p≤0.0001=****.

FIGS. 39A-39I: Effects of KLF6 enhancer disruption in hematopoietic reconstitution. (FIG. 39A) Bar plot representation of mRNA quantification after targeting E1 region or NTC in HSPCs. (FIG. 39B) T7E1 assay performed in E1 disrupted HSPCs. PCR amplicons targeting KLF6 or the E1 region were either treated (+) or untreated (−) with T7E1. While KLF6 genomic regions remains unchanged after E1 targeting, E1 region shows the corresponding cleavage sites. Arrowheads indicate the bands with expected size based on the Cas9 cleavage site. The numbers below the gel image represent the cleavage efficiency determined by densitometric analysis. (FIG. 39C) Representative flow cytometry histograms displaying KLF6 protein levels in young healthy human CD34+ cells after E1 disruption (left) and quantification of KLF6 positive cells in NTC or E1 targeted cells (right). (FIG. 39D) Representative flow cytometry plots showing peripheral blood contribution of human CD45+ in E1 KO and NTCs HSPCs at week 6 post-transplant (left) and summary of reconstitution throughout the experiment (right) (n=10 per group). Significance was assessed using a linear mixed-effects model with the parameter examined. (FIG. 39E) Representative flow cytometry plots assessing peripheral blood myeloid to lymphoid ratio observed in E1 disrupted and NTCs HSPCs at week 6 post-transplant (left) and summary of the ratio throughout the experiment (right). (n=10 per group) Significance was assessed using a linear mixed-effects model with the parameter examined. Representative FACS plot showing FMP (FIG. 39F), MEP (FIG. 39G), MPP (FIG. 39H) and lymphoid frequency (FIG. 39I) in NSGS mice transplanted with E1 KO or NTC HSCPs. Significance was assessed using a two-tailed unpaired t-test.

FIGS. 40A-40K: KLF6 restoration reverses aging like phenotypes in age HSPC in vitro. (FIG. 40A) Schematic representation of the experimental design used for assessing the effects of KLF6 restoration in young and aged HSPCs in vitro. (FIG. 40B) Representative flow cytometry histograms showing KLF6 protein levels in young and aged KLF6 induced or NTC healthy human CD34+ cells 96 hr after induction and bar plot summary for all replicates (n=3). (FIG. 40C) Bar plot representation of mRNA quantification of KLF6 in KLF6 induced or NTC young and aged healthy human CD34+ cells 96 hr after induction (n=3). (FIG. 40D) Representative flow cytometry plots of the myeloid markers CD11b and CD33 in KLF6 induced or NTC young and aged healthy human CD34+ cells, after 7 days in myeloid promoting liquid culture, and bar plot summarizing all replicates (n=3). (FIG. 40E) Volcano plot of the log 2 fold change (aged KLF6-a/aged NTC) of differentially expressed genes after KLF6 induction in aged HSPCs. Significant upregulated and downregulated genes in aged KLF6 induced HSPCs compared with aged NTC HSPCs are colored in red and blue, respectively (FDR <0.05, absolute(fold-change) >2). Select differentially expressed genes are labeled. (FIG. 40F) Bar plot representation of the normalized enrichment score (NES) for select gene enrichment pathways that are up or downregulated with KLF6 induction in aged HSPCs. (FIG. 40G) Heatmap of the GSEA gene sets that are significantly (FDR <0.1) enriched in unmanipulated aged HSCe compared to young, plotting the NES for young vs. aged HSCe, young KLF6-a vs. young NTC, and aged KLF6-a vs. aged NTC. Gene sets that were not significant (FDR>0.1) in the KLF6-a comparisons are colored grey. Select Biocarta and Reactome gene sets are labeled. (FIG. 40H) Heatmap of the core set of genes that are deregulated (FDR <0.05, ainbsolute fold-change >2) with loss of KLF6 in young HSPC and have rescued gene expression with KLF6-a in aged HSPC. The log 2foldchange is plotted for each comparison. Select genes are labeled. (FIG. 40I) Heatmap representation of regions with either loss or gain (FDR <0.05) of H3K27ac or H3K4me1 signal in aged NTC HSPC compared to aged KLF6 induced HSPC. The log 2(IP/Input) signal is plotted for each condition, centered on the differential peak+/−5 kb. Biological replicates (n=2) were pooled for plotting. (FIG. 40J) Bubble plot representation of select gene ontology biological processes (GOBP) pathways enriched in regions with increased or decreased H3K4me1 or H3K27ac after KLF6 induction in aged HSPCs. The size of each bubble in the plot corresponds to the number of genes within the cluster that are within the given gene set. Highly significant (FDR <0.05) categories are colored in yellow. (FIG. 40K) Examples of transcription factor binding DNA motifs enriched in regions that display gain of H3K27ac or H3K4me1 loss after induction of KLF6 in aged HSPCs. p-values from paired t-test, calculated with Prism, p>0.05=ns, p≤0.05=*, p≤0.01=**, p≤0.001=***, p≤0.0001=****.

FIGS. 41A-41G: KLF6 Enhancer disruption promotes aging-like features in vivo. (FIG. 41A) Schematic of the xenotransplantation of Cy3-Cas9 E1 targeted young human HSPCs. (FIG. 41B) Representative flow cytometry plots of the lymphoid CD19 marker verse the myeloid CD33 marker, in peripheral blood of NSGS mice 6 weeks after xenotransplant and quantification of these markers throughout reconstitution (n=10 per group). Representative FACS plot of the HSC (CD34+/CD38-) (FIG. 41C) and progenitor (CD34+/CD38+) (FIG. 41D) compartments in bone marrow of NSGS mice 16 weeks after transplantation with E1 KO HSPCs and quantification of these populations. (FIG. 41E) Representative flow cytometry plot of GMP specific expansion detected after E1 KO HSPCs, in bone marrow of NSGS mice 16 weeks after transplantation. p-values from linear mixed-effects model or paired t-test, calculated with R with the nlme package or Prism, p>0.05=ns, p≤0.05=*, p≤0.01=**, p≤0.001=***, p≤0.0001=****.

FIGS. 42A-42H: KLF6 induction restores aging like phenotypes in age HSPC in vitro. (FIG. 42A) Bar plot representation of mRNA quantification from KLF6 induced and aged HSCPs obtained at different timepoints of the experiment. (FIG. 42B) Representative flow cytometry histograms displaying KLF6 protein levels in aged healthy human CD34+ cells after KLF6 induction or NTC (left). Quantification of KLF6 positive cells in aged healthy human CD34+ KLF6-a or NTC cells (right). (FIG. 42C) Representative flow cytometry plots of young and aged unmanipulated human CD34+ cells cultured in myeloid promoting conditions (left). Bar plot quantification representing the differentiation potential of young and aged unmanipulated human CD34+ cells cultured in myeloid promoting conditions (right). (FIG. 42D) Colony-forming unit potential of aged NTC, aged unmanipulated, aged KLF6-a, young NTC and young unmanipulated HSPCs. Colony numbers per 500 CD34+ cells plated are plotted for total colony number, granulocyte-macrophage (GM), granulocyte-erythrocyte-macrophage-megakaryocyte (GEMM) and burst-forming unit erythroid (BFU-E). Colony numbers for each biological replicate (n=3) are normalized to the total colony number for that replicate. (FIG. 42E) Functional annotation using EnrichR Gene Ontology Biological Processes for core genes deregulated with KLF6 depletion in CD34+ cells from human young healthy donors and rescued with KLF6 induction in CD34+ cells from human age healthy individuals. (FIG. 42F) Genomic annotation of regions that exhibit either loss or gain of H3K27ac or H3K4me1 after KLF6 induction in aged CD34+ cells. (FIG. 42G) Venn diagram illustrating the number of peaks that are lost with normal aging in HSCe and have increased H3K27ac upon KLF6 induction in aged CD34+ cells. (FIG. 42H) Density plot of log 2 (pooled IP/pooled Input) H3K4me1 or H3K27ac signal for aged and young NTC as well as age and young KLF6 induced samples at regions previously shown to lose H3K4me1 (left) or H3K27ac (right) in aged HSCe.

FIGS. 43A-43F: KLF6 induction rejuvenates aged HSPCs in vivo. (FIG. 43A) Experimental design for studying the effects of KLF6 induction in aged HSPC in NSGS mice. (FIG. 43B) Representative flow cytometry plots of the lymphoid CD19 marker verse the myeloid CD33 marker, in peripheral blood of NSGS mice 6 weeks after xenotransplant and quantification of these markers throughout reconstitution (n=8 per group). (FIG. 43C-FIG. 43D) Representative FACS plot of the HSC (CD34+/CD38−) (FIG. 43C) and progenitor (CD34+/CD38+) (D) compartments in bone marrow of NSGS mice 16 weeks after transplantation with aged KLF6 induced HSPCs and quantification of these populations (n=8 per group). (FIG. 43E-FIG. 43F) Representative FACS plots of the MEP (FIG. 43E) and MPP (FIG. 43F) populations in bone marrow of NSGS mice 16 weeks after transplantation with aged KLF6 induced HSPCs and quantification of these populations (n=8). p-values from linear mixed-effects model or paired t-test, calculated with R with the nlme package or Prism, p>0.05=ns, p≤0.05=*, p≤0.01=**, p≤0.001=***, p≤0.0001=****.

FIGS. 44A-44H: KLF6 activation rejuvenates aged HSCs in vivo. (FIG. 44A) Bar plot representation of mRNA quantification from peripheral blood aged healthy KLF6 induced human CD45+ cells and aged NTC human CD45+ obtained at different timepoints of the experiment. (FIG. 44B) Necropsy analysis of 16-week NSGS mice injected with KLF6 induced or NTC aged HSCPs. Representative photographs of liver, spleen and pancreas of 16-week NSGS mice injected with KLF6 induced or NTC aged HSPCs. (FIG. 44C) Liver (left), spleen (middle) and pancreas (right) weighs of 16-week NSGS mice injected with KLF6 induced or NTC aged HSCPs. (FIG. 44D) Total body weights of 16-week NSGS mice injected with NTC or KLF6-a HSCPs. (FIG. 44E) Representative flow cytometry plots assessing peripheral blood myeloid to lymphoid ratio at week 6 post-transplant of KLF6 induced and NTCs aged HSPCs (right) and summary of observed ratios throughout the experiment (left). Significance was assessed using a linear mixed-effects model with the parameter examined. (FIG. 44F) Representative flow cytometry plots showing GMP (FIG. 44F), CMP (FIG. 44G) and lymphoid (FIG. 44H) fractions in NSGS mice transplanted with KLF6-a or NTC aged HSCPs. Significance was assessed using a two-tailed unpaired t-test.

FIGS. 45A-45E: KLF6 modulates aging associated phenotypes in human HSPCs. (FIG. 45A) Representative immunofluorescence (IF) micrograph showing γH2AX and 53BP1 double strand breaks markers in young human KLF6 KO, E1 KO or aged KLF6-a HSPCs harvested from NSGS mice 16 weeks after transplant and bar plot summary of all replicates (n=3). (FIG. 45B) 4′,6-diamidino-2-phenylindole (DAPI) staining representing nuclear [symbol] morphological changes in young KLF6 depleted or aged KLF6 induced HSPCs. harvested from NSGS mice 16 weeks after transplant and bar plot summary of all replicates (n=3). (FIG. 45C) SA-β-galactosidase staining in young KLF6 depleted or aged KLF6 induced HSPCs. harvested from NSGS mice 16 weeks after transplant and bar plot summary of all replicates (n=3). (FIG. 45D-FIG. 45E) Representative flow-cytometry plots of total or mitochondrial reactive oxygen species (ROS) in young KLF6 depleted or aged KLF6 induced HSPCs harvested from NSGS mice 16 weeks after transplant and bar plot summary of all replicates (n=3).

FIGS. 46A-46G: KLF6 is required for modulating aging associated phenotypes in human cells. (FIG. 46A) Representative immunofluorescence (IF) micrograph showing γH2AX and 53BP1 double strand breaks markers 6 weeks post-injection of young KLF6 KO, E1 KO or aged KLF6-a HSPCs in NSGS mice. (FIG. 46B) 4′,6-diamidino-2-phenylindole (DAPI) staining representing nuclear morphological changes in young KLF6 depleted or aged KLF6 induced HSPCs 6 weeks post-injection in NSGS mice. (FIG. 46C) SA-β-galactosidase staining in young KLF6 depleted or aged KLF6 induced HSPCs 6 weeks post-injection in NSGS mice. (FIG. 46D) Bar plot representation of p16 mRNA quantification of in young KLF6 KO, KLF6 enhancer targeted and aged KLF6 induced HSPCs 6- and 16-weeks post-injection in NSGS mice. Bar plot quantification of total (FIG. 46E) or mitochondrial (FIG. 46F) reactive oxygen species (ROS) assessment in young KLF6 depleted or aged KLF6 induced HSPCs 6 weeks post-injection in NSGS mice. (FIG. 46G) Cell cycle distribution quantification of young KLF6 depleted or aged KLF6 induced HSPCs 6- and 16-weeks post-injection in NSGS mice.

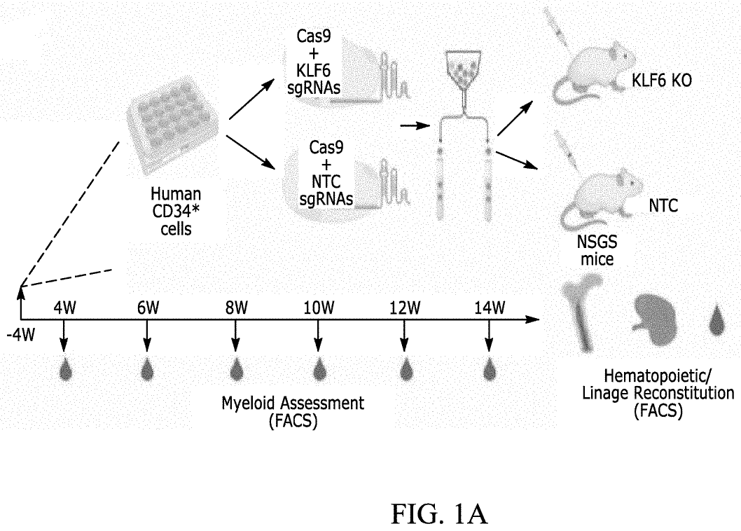

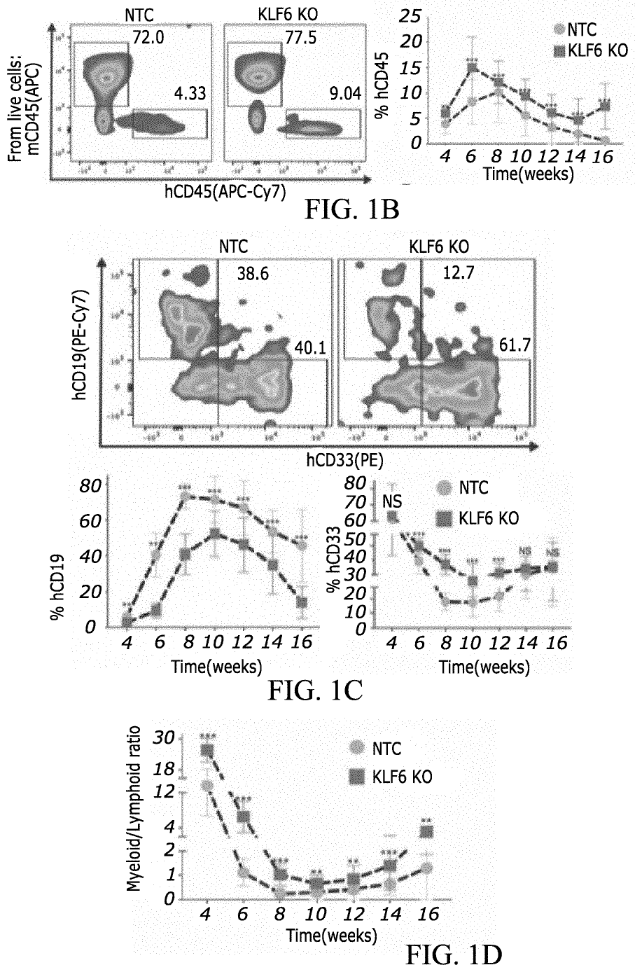

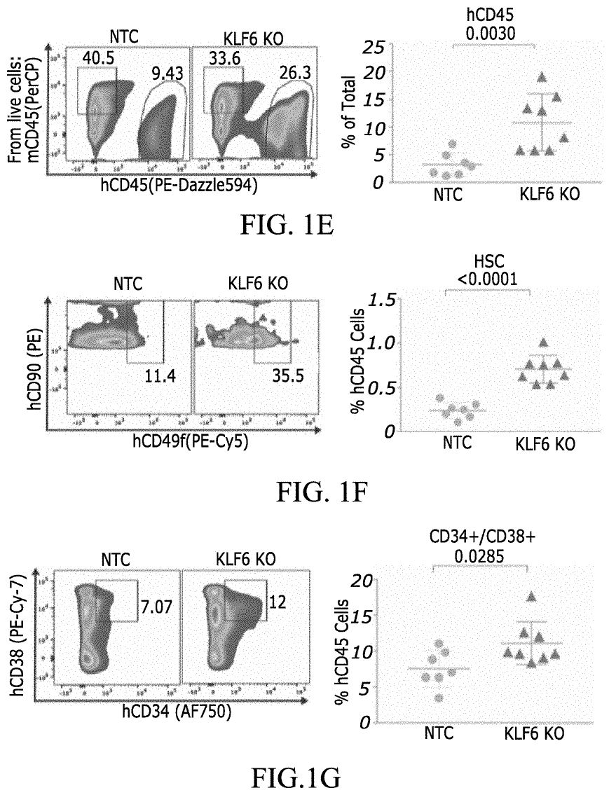

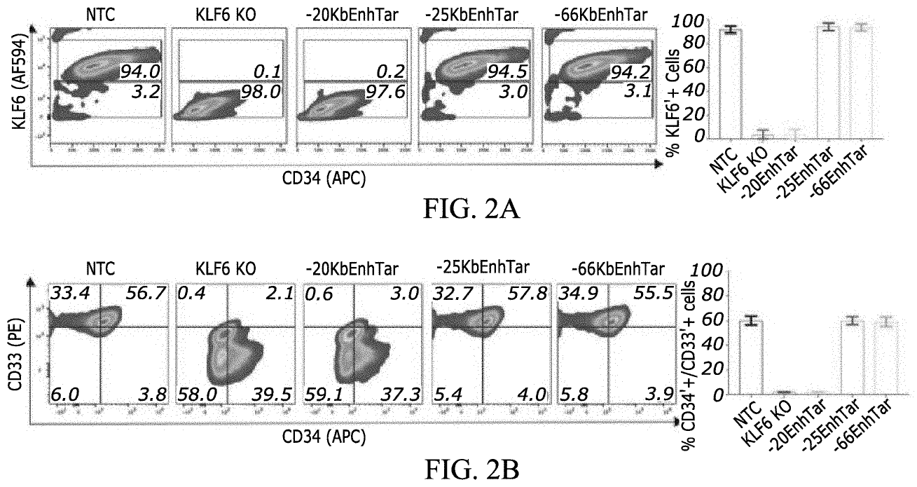

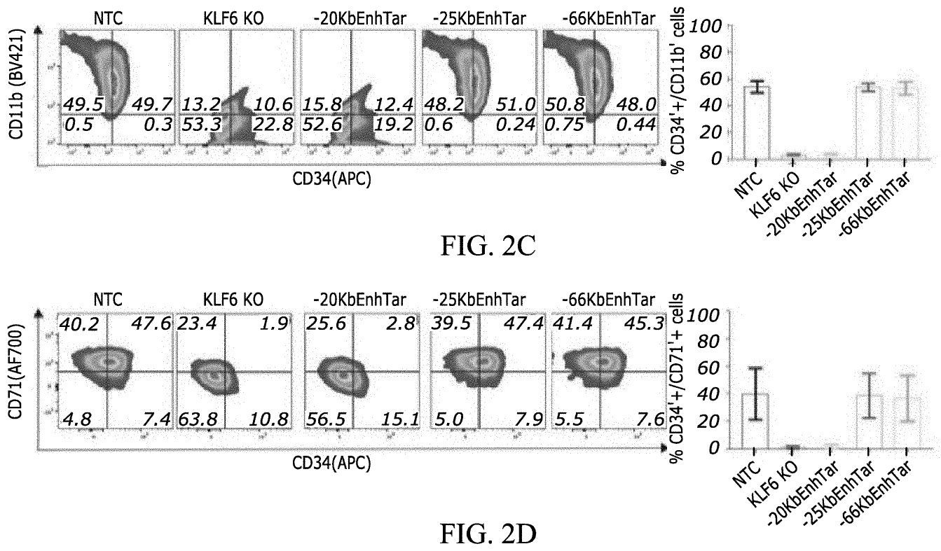

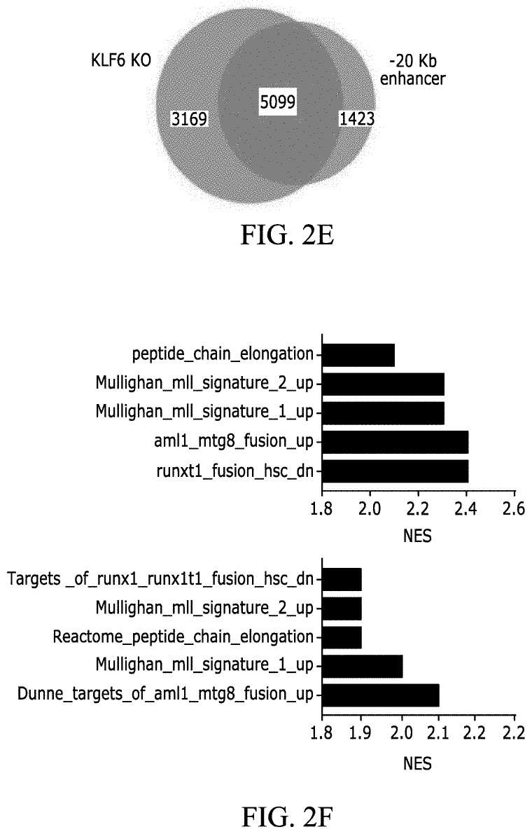

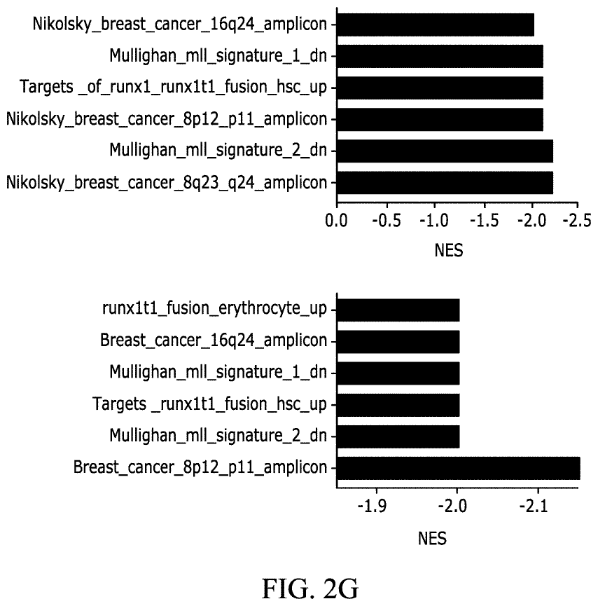

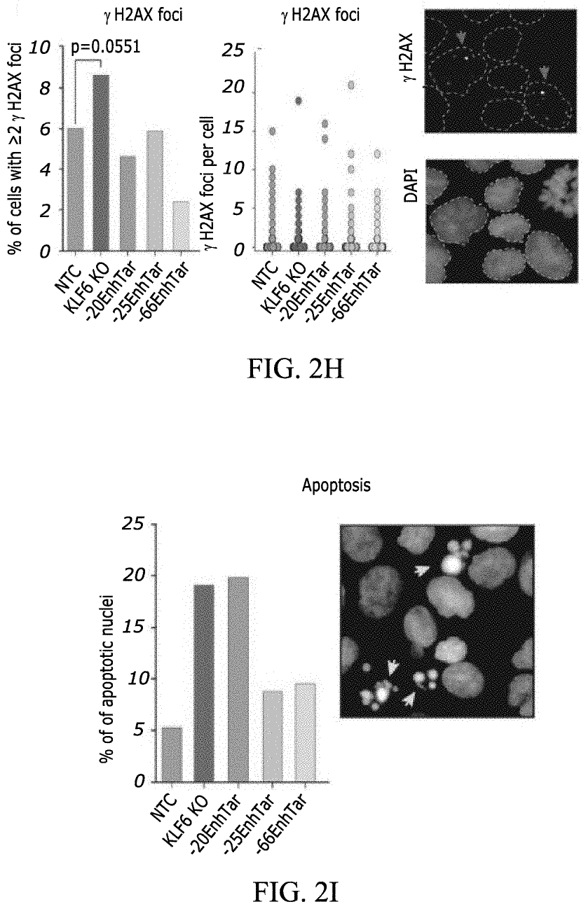

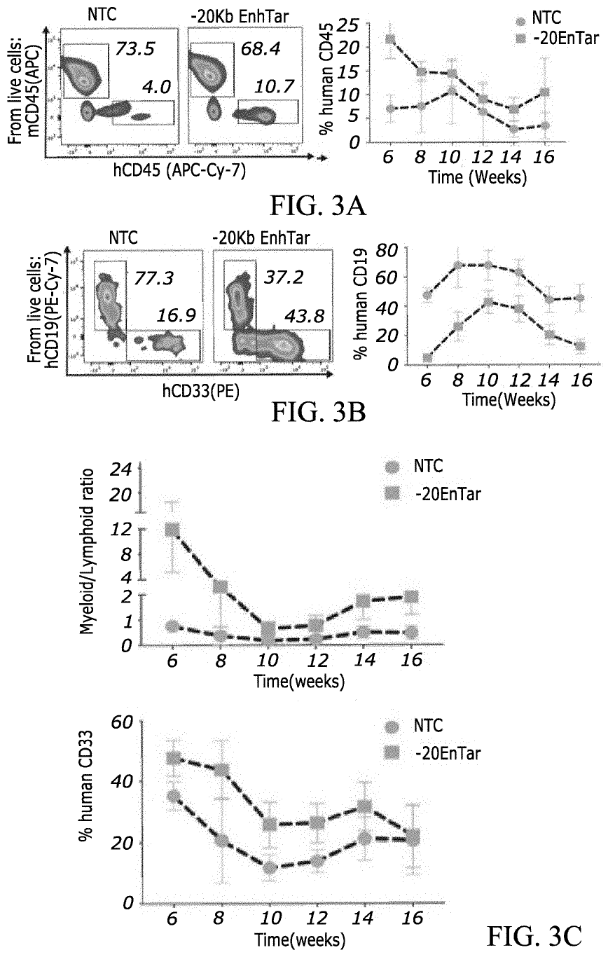

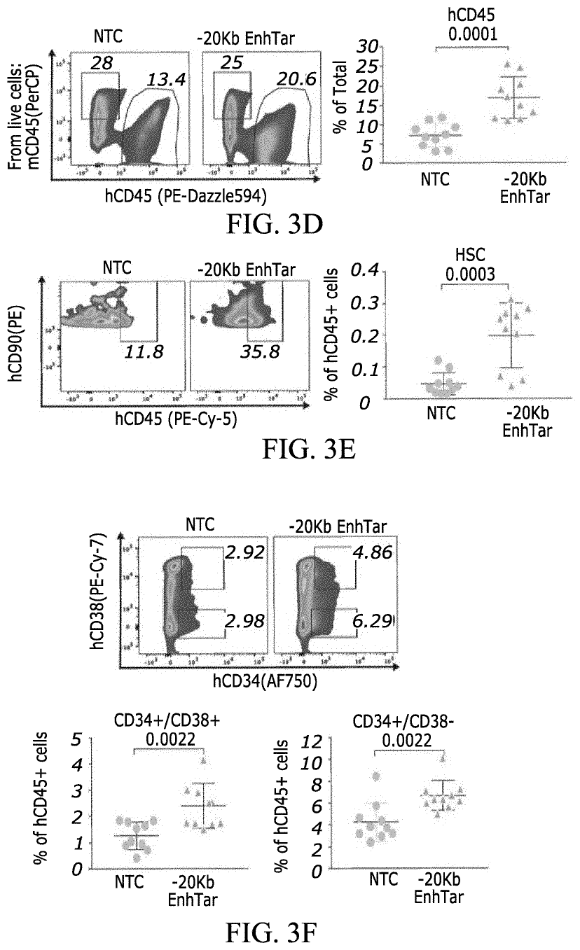

### DETAILED DESCRIPTION

HSC first originate in the aorta gonad mesonephros in mammals and after passing through the liver they lastly home the bone marrow (BM) (Medvinsky and Dzierzak, 1996; Ema and Nakauchi, 2000). Along with this, the conception that cells experience a unidirectional evolutionary process throughout development has been proved inaccurate by the evidence that differentiated cells can be reprogramed to a pluripotent state factors (Takahashi and Yamanaka, 2006; Takahashi et al, 2007) and occurring through the modification of epigenetic marks (Buganim et al., 2012, 2013; Hansson et al., 2012). That main epigenetic components affected during aging are restructured during reprograming (Ocampo et al, 2016; Lapasset et al., 2011; Liu et al., 2011; Mahmoudi and Brunet, 2012; Rando and Chang, 2012). HSC exhibit an extensive epigenetic reprogramming with aging resulting in the loss of key regulatory regions. This age associated epigenome rewiring of HSPC affects the transcriptional profiles of many genes and transcription factors (TF). The Krüppel-like factors 6 (KLF6) was amongst the top deregulated TFs; further characterization of KLF6 in HSC lead to identification of key functional properties as regulator of normal hematopoiesis.

Kruppel-like factors (KLFs) are evolutionary conserved DNA-binding transcriptional regulators that perform diverse roles during proliferation, development, differentiation and signal transduction (Jiang et al, Nature Cell Biol, 10(3), 353, 2008; McConnell et al, Physiol. Rev., 90(4), 1337, 2010; Yamanaka et al, Cell, 131(5), 861, 2007). Changes in their functions have been associated with the pathobiology of numerous diseases, including cardiovascular disease, metabolic disorders and cancer. KLF6, the most strongly downregulated member of the family in HSPC with aging, is ubiquitously expressed in normal tissues and its expression is lost in many cancers. For example, in head and neck squamous cell cancer, hepatocellular carcinoma, non-small-cell lung cancer prostate cancer and renal carcinoma, KLF6 negatively regulates cell proliferation by upregulating CDKN1A, activates CDKN1B in NSCLC, and downregulates CCND1 in HCC and melanoma (Lang et al, 2013; Li et al, 2005; Narla et al, 2005; Sangodkar et al al, 2099; Tahara et al, 2009; Teixeira et al, 2007). Moreover, Klf6 is required for yolk sac hematopoiesis and Klf6−/− mice are embryonic lethal (Matsumoto et al, 2006). Klf6−/+ mice have increased liver tumor formation and decreased p53 protein levels due to Klf6 transcriptionally repressing the p53-specific E3 ubiquitin ligase Mdm2 (Tarocchi et al, 2011). In terms of tissue remodeling and regeneration, KLF6 enhances vascular repair the common femoral artery (Gallardo-Vara et al., 2016; Garrido-Martin et al., 2013), promotes axon regeneration in the central nervous system (CNS) (Wang et al, 2018) and is required for CNS myelination (Laitman et al, 2016).

KLF6 is essential in HSC biology and KLF6 depleted cells exhibit a differentiation impairment in vitro and recapitulate aging and malignant profiles. As described further herein, age acquired epigenetic deregulation and loss of KLF6 expression is associated with age related changes in HSPC function and increased risk of malignant transformation. Surprising, KLF6 induction in a normal ageing background re-establishes KLF6 transcriptional levels and adjusts the HSPC aging phenotype, generating HSPC with improved characteristics for bone marrow transplantation.

The disclosure provides method of preparing HSPC for bone marrow transplantation, the method comprising (a) obtaining donor hematopoietic stem and progenitor cells and (b) upregulating expression of Kruppel-like factor 6 (KLF6). In various aspects, method further comprises (c) administering the HSPC of step (b) to a subject in need thereof.

Hematopoietic stem and progenitor cells (HSPCs) are a stem cell lineage that gives rise to all blood cell types, including erythroid (erythrocytes or red blood cells (RBCs)), myeloid (monocytes and macrophages, neutrophils, basophils, eosinophils, megakaryocytes/platelets, and dendritic cells), and lymphoid (T-cells, B-cells, NK-cells). HSPC from peripheral blood can be collected using, e.g., an apheresis machine. HSPC from bone marrow can be obtained, e.g., directly from bone marrow from the posterior iliac crest by needle aspiration (see, e.g., Kodo et al., 1984, J. Clin Invest. 73:1377-1384), or from the blood following pre-treatment with mobilizers, such as cytokines (e.g., G-CSF and/or AMD3100). HSPC can be positive for any of a series of markers preferentially expressed on HSPC relative to other types of hematopoietic cells. Such markers include, but are not limited to, CD34, CD43, CD45RO, CD45RA, CD49f, CD59, CD90, CD109, CD117, CD133, CD166, and HLA DR. Alternatively or in addition, the HSPC can be negative for a marker relative to other types of hematopoietic cells including, e.g., Lin, CD38, or a combination thereof. In various aspects, the cells are CD34+ HSPCs. In various aspects, the donor HSPC population is autologous with respect to an intended transplant recipient. In another embodiment, the donor HSPC population is allogeneic with respect to an intended transplant recipient.

The method comprises upregulating (or activating) expression of KLF6 in a population of HSPCs. KLF6 expression may be upregulated in any suitable manner. In various aspects, step (b) comprises contacting the HSPC with CRISPR/dCas9 fused to an activation domain. CRISPR/dCas9-mediated gene activation (also known as CRISPRa) systems comprise a dCas9-activation domain fused to a transcription activation domain and sgRNA, which target a particular sequence of interest (e.g., a the specific promoter or enhancer region). An exemplary activation domain is the VP64 acidic transactivation domain (four copies of Herpes simplex virus protein 16). CRISPRa systems comprising VP64 have been shown to activate reporter genes and endogenous genes with a single sgRNA. Other suitable CRISPRa systems include, but are not limited to, the dCas9-SunTag system, the dCas9-CPR system, dCas9-VP64/P65/Rta systems, as well as systems employing P65AD, VP65/MS2/P65/HSF1 domains, VPR/PYL1 domains, VPR/GIDI domains, VPR/P65 domains, and the like. Effector domains may also be fused to or incorporated into the sgRNA scaffold; exemplary sgRNA-activation domain constructs include those comprising MS2 hairpin aptamers. CRISPRa systems are further described in, e.g., Adli, Nature Communications (2018) 9:1911; and Xu and Qi, J Mol Bio (2019) 431:34, as well as Javaid et al., Int J Mol Sci. 2021 January; 22(1): 397 (incorporated by reference herein).

The disclosure contemplates use of the materials and methods described herein to, e.g., improve the clinical outcome of hematopoietic stem and progenitor cells (HSPC) transplantation and/or expand the pool of available donors for bone marrow transplantation.

Materials and Methods

Cell culture: Human CD34+ hematopoietic stem and progenitor cells from mobilized peripheral blood of anonymized healthy donors were obtained from Fred Hutchinson Cancer Research Center, Seattle, Wash. CD34+ HSPCs were thawed on day 0 and cultured in stem-cell-promoting media (IMDM+20% Human Serum AB supplemented with human stem cell factor (SCF) (100 ng/ml), human thrombopoietin (TPO) (100 ng/ml), recombinant human Flt3-ligand (Flt3-L) (10 ng/ml), human interleukin 6 (hIL-6) (20 ng/ml) and non-essential amino acids) under low oxygen conditions to maximize maintenance and expansion of LT-HSCs (Danet el al, 2003; Koller et al, 1993; Bak et al 2017).

RNP electroporation: Guide RNAs were produced as described before (Adelman et al, 2019). Briefly, protospacer sequences were identified using the CRISPRdesign algorithm (crispr.mit.edu) or CRISPRScan (www.crisprscan.org) (Moreno-Mateos et al, 2015). DNA templates for sgRNAs contain a T7 promoter, the protospacer sequence, and the sgRNA scaffold sequence (Gundry et al 2016). They were produced by PCR using custom forward primers and a reverse primer that amplifies the sgRNA scaffold of the plasmid pKLV-U6gRNA-PGKpuro2ABFP (Addgene #62348). PCR products were purified and in vitro transcribed with the HiScribe T7 High Yield RNA Synthesis Kit (NEB #E2040S) following manufacturer instructions. In vitro transcribed sgRNA products were purified using RNA Clean & Concentrator kit (Zymo Research #R1015). Modified synthetic sgRNAs (2′-O-methyl-3′-phosphorothioate modifications in the first and last three nucleotides) were from Synthego.

After 48-72 hs in culture, HSPC were electroporated with Cy3-Cas9 protein (PNA Bio, CP06) and sgRNAs using the Neon Transfection System (Thermo Fisher). Transfection conditions were 1600 volts, 10 milliseconds, and 3 pulses. After transfecting, HSPCs were cultured in stem-cell-promoting media for 24 hs under low oxygen conditions (Danet el al, 2003; Koller et al, 1993; Bak et al 2017) before sorting for the Cy3-Cas9+/CD34+ population. After sorting cells were maintained for 72 hs before performing the functional assays described below.

Clonal culture of CD34 and indel frequencies: Edited CD34+HSCPs were sorted into 50 ul of MethoCult H4435 (StemCell Technologies, #04435), in 96-well round-bottom plates (Nunc) at one cell per well using BD FACS SORP Aria Fusion (BSL-2). After additional 18-24 days of culture cell were washed 6-8 times in PBS 1× and viability was assessed by trypan blue exclusion. Genomic DNA was extracted from those colonies with viability greater that 80% using the Qiagen Blood and Tissue kit. KLF6 −20 KB enhancer was amplified with KOD Hot Start DNA polymerase and corresponding primers using the following cycling conditions: 95° C. for 3 min; 35 cycles of 95° C. for 20 s, 60° C. for 10 s, and 70° C. for 10 s; 70° C. for 5 min. Resulting PCR products were subjected to Sanger sequencing. Sequencing traces were imported to TIDE software for indel frequency measurement with 40 bp decomposition window.

Lentiviral production and of CD34+ transduction: 293FT cells were maintained according to supplier instructions (ThermoFisher Scientific). dCas9-VP64 (addgene #53192) lentiviral particles were produced by co-transfection with packaging plasmids psPAX2 and pMD2.G using polyethylenimine transfection reagent (Polysciences). Lentivirus containing supernatant was collected 48-72 hours post-transfection, filtered through a 0.45-μm syringe filter, and concentrated using PEG-it virus precipitation solution (System Biosciences, #LV825A-1). Primary human CD34+ cells were freshly isolated from mobilized peripheral blood. Lentiviral transduction of CD34+ cells was performed in the presence of 8 μg/ml polybrene (Millipore Sigma, TR-1003-G). Four days post transduction, cells were sorted for CD34 and GFP double-positive cells on a BD FACS SORP Aria Fusion (BSL-2). After sorting, cells were cultured in stem-cell-promoting media (IMDM+20% Human Serum AB+hTPO (100 ng/ml), hSCF (100 ng/ml), hIL-6 (20 ng/ml), hFLT-3 (10 ng/ml) and non-essential amino acids) under low oxygen conditions for 48-72 hs where sgRNAs were transfected (Thakore et al, 2015). Cells were cultured for an additional 24-48hs prior to follow-up in vitro analysis. Guide RNA sequences were designed using the crispr.mit.edu web-based tool, targeting them to the proximal promoters (−250 to −50 base pairs from transcription start site) of the gene of interest. Possible guides were selected according to their off-target score and position. A non-targeting sgRNA was used as control. After 24-48hs in culture, cells were used for RNA extraction, CFU assay, or plated in myeloid: SCF (100 ng/ml), FLT-3 ligands (10 ng/ml), IL-3 (20 ng/ml), IL-6 (20 ng/ml), GM-CSF (20 ng/ml), and G-CSF (20 ng/ml) and/or erythroid-promoting conditions: Epo (6 IU/ml) and SCF (100 ng/ml). At day 7 of myeloid and erythroid expansion, cells were stained for CD34 (BD Pharmigen, #304441), myeloid CD11b (BD Pharmigen, #301324), and erythroid CD71 (Pharmigen, #563769) and CD235a (BD Pharmigen, #559943) markers respectively.

RNA-seq: RNA from transfected CD34+ cells (n=3 biological replicates and 1-3 technical replicates for a total of n=4 sgCTRL and n=4 sgKLF6) was extracted using the Qiagen Allprep Micro kit according to manufacturers instructions (Qiagen, #80204). Stranded libraries were prepared using the Illumina TruSeq Stranded Total RNA kit (Illumina, #20020596). Libraries were sequenced on the HiSeq-3000 with 75 bp paired-end sequencing. Data was aligned and processed as described above. For GSEA, the Wald statistic ranked list was used with the top (ranked by log 2FoldChange) 500 genes up- and down-regulated with HSCe aging as gene sets as well as the c2.all.v6.2. symbols gene set using the weighted enrichment score.

Differential gene expression analysis: Gene counts were calculated using QoRTs (v1.0.7). QoRTs was run in second stranded mode using the hg19 gencode annotation file without entries for ribosomal RNA. Differential gene expression analysis was performed using DESeq2 v1.10.1. A multifactor design was used in order to control for sex of the donor as well as any batch effect during library preparation. Dispersions were calculated using samples from both age groups and then contrasts were established for pair-wise comparisons. Significant genes were defined as having a fold change >1.5 and p-adjusted <0.05. Regularized log-counts (rld) were generated with DESeq2 and then row z-scores were calculated and used to plot heatmaps using the R package ComplexHeatmap (v1.15.1) with average clustering and correlation distances.

GSEA: Gene set enrichment analysis (GSEA) was performed using a list of genes pre-ranked by the Wald statistic (stat column from DESeq2 output). A weighted enrichment score was used and gene set size was limited to 15-500 genes. To test enrichment for the Crews et al. aging signature, the published list of genes upregulated in aged HSCe (FPKM>1, p<0.05, L2FC >1) was used as a gene set in GSEA.

Myeloid and erythroid liquid culture: Cells were plated and cultured for 7 days under myeloid-promoting conditions: SCF (100 ng/ml), FLT-3 ligands (10 ng/ml), IL-3 (20 ng/ml), IL-6 (20 ng/ml), GM-CSF (20 ng/ml), and G-CSF (20 ng/ml) and erythroid-promoting conditions: Epo (6 IU/ml) and SCF (100 ng/ml). At day 7 of myeloid and erythroid expansion, cells were stained for CD34 (BD Pharmigen, #304441), myeloid CD11b (BD Pharmigen, #301324), and erythroid CD71 (Pharmigen, #563769) and CD235a (BD Pharmigen, #559943) markers respectively, and also anti-KLF6 for cells transfected with sgKLF6 (Millipore, #MABN119).

Colony-forming unit assay: Sorted CD34+ were seeded in methylcellulose, MethoCult H4435 (StemCell Technologies, #04435), in duplicate onto a 6-well SmartDish (StemCell Technologies, #27302) at varying densities of 500 cells per condition. Colonies were scored on a STEMvision after 14 days of incubation (StemCell Technologies).

ChIP-seq: HSCe were sorted into 1 mL IMDM 20% FBS. For H3K4me1 and H3K4me3 HSCe were used per immunoprecipitation; for H3K27ac, 40,000 cells were used per immunoprecipitation. ChIP-seq was then performed using the True MicroChiP (Diagenode, #C01010130) kit and that had been validated antibodies for specificity and reactivity using the MODified Histone Peptide Array (Active Motif, #13001). The manufacturer's protocol was followed using the following modifications. After quenching with glycine and washing with PBS, samples were suspended in 1001 undiluted Lysis buffer with 1× Diagenode protease inhibitor cocktail and 5 mM sodium butyrate per 10,000 cells. Samples used for H3 were sonicated in 1.5 mL TPX tubes in a Bioruptor Pico for 6 cycles of 30 seconds on and 30 seconds off. All other samples were sonicated in a Bioruptor XL for 55 cycles of 30 seconds on and 30 seconds off. Chromatin was immunoprecipitated for 12 hr at 4° C. using 1 [symbol] g H3K27me3 (Millipore 07-449, lot #21494165), 1 [symbol] g H3K4me3 (Abcam ab8580, lot #GR164207-1), 0.5 [symbol] g H3K4me1 (Diagenode C15410194, lot #A1862D), 0.5 [symbol] g H3K27ac (Abcam ab4729, lot #GR155970-2), or 0.5 [symbol] g H3 (Abcam ab10799, lot #GR275925-1). After reverse crosslinking, DNA was purified using the minElute PCR Purification kit (Qiagen, #28004) and eluted in 161 of Tris-HCl ph 8.0. Enrichment was verified using QPCR with the primers GAGAGTCCTGGTCTTTGTCA (SEQ ID NO: 1) and ACAGTGCCTAGGAAGGGTAT (SEQ ID NO: 2) for H3K4me1 and H3, AGGGAGGGAATTAATCTGAG (SEQ ID NO: 3) and ACAGTGCCTAGGAAGGGTAT (SEQ ID NO: 2) for H3K4me3 and H3, TACTTGGTTTCTGCATCCTT (SEQ ID NO: 4) and TCACTAAAGAAACCGTTCGT (SEQ ID NO: 5) for H3K27me3 and H3, and GAGCAGAGGTGGGAGTTAG (SEQ ID NO: 6) and TCAGACCCTTTCCTCACC (SEQ ID NO: 7) for H3K27ac. The remaining DNA was then used for library preparation with the V1 MicroPlex Library Preparation kit (Diagenode, #C05010011). For the PCR amplification, a total of 16 amplification cycles was used. Libraries were purified using a 1:1 Ampure bead cleanup and eluted in 16 uL of Tris-HCl ph 8.0. Fold enrichment over input was then verified using QPCR (primer sequences in Supplementary Table SX). Multiplexed libraries were sequenced either on an Illumina NextSeq 500 or a HiSeq-2500 sequencer.

Results

Loss of KLF6 in HSC Recapitulates Physiological Hematopoietic Aging In Vivo

A study was conducted to assess impact that loss of KLF6 has on impairment of normal hematopoiesis in vivo. CRISPR-Cas9 genomic disruption was performed using Cas9 protein pre-complexed with sgRNAs to produce a ribonucleoprotein particle and target the KLF6 locus in wild type primary human CD34+ cells isolated from healthy young donors (26-28 years of age) (Kim et al., 2014; Lin et al., 2014; Schumann et al., 2015). CRISPR-Cas9 mediated disruption of KLF6 in HSPCs resulted in efficient deregulation of KLF6 expression both at the mRNA and protein levels. Moreover, Sanger sequenced single cell clones further confirmed the high rate of indels generated with the CRISPR-Cas9 genome editing approach used and showed that the most common mutations introduced in the targeted region were a 4 bp deletion as produced by imprecise non homologous end joining repair pathway (NHEJ).

CD34+ cells from young healthy donors transfected with either KLF6-sgRNA (henceforth denoted to as KLF6 KO) or non-targeting control (NTC)-sgRNA (henceforth denoted to as NTC) were injected into immunodeficient NSG-SGM3 (NOD.Cg-PrkdcscidII2rgtm1WjlTg(CMV-IL3,CSF2,KITLG)1Eav/MloySzJ) mice and monitored closely for engraftment.

Kinetics examination of peripheral blood engraftment (measured as percent of human CD45+ cells) showed that at week 4, continuously until the end of the experimental timepoints assessed, >1% human cells were present in both the test and the control groups. Moreover, lineage reconstitution analysis showed a significant decrease in peripheral blood between weeks 4 and 16 post-injection in the lymphoid fraction (hCD45+, CD19+, CD33−) with a concomitant increase in the myeloid output (hCD45+, CD19−, CD33+) in the mice injected with KLF6 KO cells relative to the NTC, and an increased myeloid to lymphoid ratio (CD33+ to CD19+)

CD34+ and CD34+/CD38− bone marrow populations are both heterogenous compartments where only a minor fraction of cells corresponds to stem and progenitor hematopoietic cells. Consequently, to better understand and distinguish changes driven by KLF6 disruption in the bone marrow, the HSC compartment (CD34+, CD38−, CD45RA−, CD90+, CD49f+) (Notta et al, 2011) of specimens injected with KLF6 KO and NTC cells was analyzed. Interestingly, specimens in which KLF6 KO cells were injected displayed an expansion of the HSC compartment as well as an expansion in the progenitor output (CD34+, CD38+, CD45RA−, CD90+, CD49f+). An increase in the multipotent progenitors (MPPs) (CD34+CD38−CD90−CD45RA−CD49f−), common myeloid progenitors (CMPs) (CD34+CD38+CD10−FLT3+CD45RA−) and megakaryocyte-erythroid progenitor (MEPs) (CD34+CD38+CD10−FLT3-CD45RA−) was detected.

The link between age-related downregulation of KLF6 and the epigenetic reprogramming observed in the elderly also was examined. Using a low-input protocol for chromatin immunoprecipitation followed by massively parallel sequencing (micro ChIP-seq), the genome-wide distribution of H3K4me1, H3K27ac and H3K4me3 was evaluated after KLF6 depletion in human HSPCs. Differential peak calling analysis for H3K27ac and H3K4me3 revealed that KLF6 depleted HSPCs displayed both gains 11,282 (42.6% of differential peaks) and 11,072 (59.9% of differential peaks) and losses 15,171 (57.3% of differential peaks) and 7,404 (40% of differential peaks), respectively (log 10 likelihood ratio >3, absolute fold-change ≥1.5). Functional annotation in the histone profiles revealed that sites with reduced H3K4me3 were associated with genes implicated in cancer and catabolic processes, while loss of H3K27ac was linked to genes involved in hematopoietic differentiation, similarly of what is been documented during HSC aging. Additionally, regions that gained H3K27ac in the KLF6 depleted HSPCs were associated with age-associated features, such as inflammation and immune signaling pathways, while regions that gained H3K4me3 were enriched in genes involved in cancer and aging related diseases such as Alzheimers. The degree of similarity between regions that display loss of these chromatin marks with normal human aging in the HSPC compartment and with KLF6 disruption was also explored. Regions that lose H3K27ac in both normal human aging and with KLF6 depletion are associated with hematopoiesis and many important epigenetic regulators. Moreover, regions that show decrease in H3K4me3 intensity with normal aging were compared with KLF6 KO, genes involved in differentiation commitment and development were identified. Together these results suggest that KLF6 is a key transcription factor involved in modulating normal HSPC developmental and differentiation programs, and that KLF6 deficient HSPC cells lead to the production of permanent altered HSPC, characterized by the presence of a myeloid-biased population and an increment of stem and progenitor populations.

KLF6 is Regulated by an Enhancer Region Found 20 kb Upstream of its Promoter in HSPCs

Transcriptional enhancers can regulate cell-type specificity and development, with many human malignancies arising from altered enhancer function (Williamson et al, 2011; Visel et al, 2009). A study was conducted to elucidate how KLF6 is transcriptionally regulated in HSPCs. Distal regulatory elements that may be responsible for this downregulation were explored. By evaluating the chromatin architecture around the KLF6 locus, three enriched regions displaying co-occupancy of H3K4me1 and H3K27ac at −20, −25 and −66 kb from KLF6 promoter were identified. Notably, all three of these putative enhancer regions displayed age-related decrease in H3K27ac. Analysis of KLF6 mRNA and protein levels after CRISPR-mediated disruption of these three enhancer regions demonstrated that the putative enhancer region located at −20 kb from KLF6, but not the −25 kb nor the −66 kb regions resulted in reduced expression of KLF6 mRNA and protein levels. In order to determine if a CRISPR-Cas9 genome editing approach at these three KLF6 putative enhancer regions was sufficient to recapitulate the in vitro differentiation impairment observed upon KLF6 disruption, the myeloid and erythroid differentiation potential of the targeted cells in liquid culture was examined. Enhancer editing at the −20 kb region upstream KLF6 was sufficient to recapitulate both the in vitro myeloid as well erythroid differentiation impairment potential as seen in KLF6 KO cells. In contrast, neither disruption of the −25 kb or the −66 kb putative KLF6 enhancers resulted in any phenotypic consequences in in the differentiation outputs of the edited cells. Likewise, enhancer disruption at −20 kb region caused an increased in both total colony numbers and granulocyte-monocyte colonies when plated on methylcellulose, demonstrating that disrupting this region in HSCP induces an increase in the myeloid colony-forming output as observed in KLF6 edited cells.

Transcriptional outputs of the two edited population were characterized. RNA-seq analysis of cells with disruption of the −20 kb enhancer to NTC displayed 6,522 differentially expressed genes (DEG) (3,288 upregulated and 3,285 downregulated). Comparison of these transcriptional changes to those seen after KLF6 KO showed a significant overlap (p value <0.0001) with 5,099 genes in common. Transcriptional profiling of both KLF6 KO and the −20 kb enhancer disrupted region confirmed a recapitulation enrichment of gene sets associated with malignant profiles, as well as with an enrichment of genes in our previously reported human HSCe aging signature, indicating that loss of KLF6 KO or the −20 kb enhancer results in an differentiation impairment and malignant transcriptional phenotypes.

Finally, HiC-seq analysis in normal human CD34+ cells confirmed interaction between the −20 kb enhancer and the KLF6 promoter, but not with the −25 kb or the −66 kb regions.

DNA damage also was assessed and quantified to determine if absence of KLF6 was sufficient to recapitulate the increase in DNA damage and apoptosis that is commonly observed in the elderly. By targeting KLF6 and the three different putative enhancers regions located at −20 Kb, −25 Kb and −66 Kb, it was observed that KLF6 targeted cells showed increased levels of S139 phosphorylated histone H2AX (γH2AX).

KLF6 Enhancer Disruption Promotes Aging Like Features In Vivo

The effect of targeting the −20 Kb KLF6 enhancer in recapitulating the phenotypic consequences observed upon targeting KLF6 in vivo was characterized. CRISPR-Cas9 enhancer editing was performed at the −20 Kb region in primary human CD34+ cells isolated from healthy young donors (26-28 years of age). An efficient deregulation of KLF6 expression was observed both at the mRNA and protein levels. CD34+ cells with disruption at the KLF6 −20 Kb enhancer (hereafter denoted to as −20 KbEnTar) or NTC were injected into immunodeficient NSG-SGM3. Peripheral blood engraftment was observed continuously through the different time points of the experiment observing >1% human cells were present in both the test and the control groups. When the linage reconstitution output of both populations was evaluated, a significant reduction in peripheral blood between weeks 6 and 16 post-injection in the lymphoid fraction (hCD45+, CD19+, CD33−) was detected with an associated increment in the myeloid production (hCD45+, CD33− CD19+) in the −20 KbEnTar injected specimens relative to the NTC. When the myeloid to lymphoid proportion reconstitution in both populations was analyzed, a significant increment in the specimens injected with −20 KbEnTar CD34+ cells was detected. As previously shown for KLF6 KO xenotransplants, bone marrow was isolated for human xenograft analysis 16 weeks post-engraftment and as expected, −20 KbEnTar recipients display an expansion of the HSC compartment in comparison with the NTC specimens. In addition, an increase in the progenitor fraction was observed, as well as in the GMP compartment in those animals injected with −20 KbEnTar. Also, on average, an increment in different progenitor compartments was observed, although these differences did not have significantly enrichment differences.

KLF6 Induction in Aged HSCs Improves Phenotypic Changes Associated with HSC Aging

Genome editing using CRISPR-Cas9 allowed evaluation of the effects of KLF6 disruption in CD34+ obtained from young, healthy donors. Next, a study was conducted to determine if re-establishing KLF6 transcriptional levels could adjust HSPC aging (i.e., counteract the negative effects of age on HSPC function and quality). For this study, a system based on a catalytically deactivated Cas9 (dCas9) fused to the VP64 transcriptional activator was employed. Lin− CD34+ cells were isolated from the bone marrow of healthy individuals aged either 65-75 yr. or 18-35 yr. After lentiviral transduction with the pLV hUbC-dCas9 VP64-T2A-GFP (herein, dCas9-VP64; Addgene #53192) (Kabadi et al, Nucleic Acid Res, 42(19), e147, 2014), the KLF6 promoter was targeted for reactivation through transfection of sgRNAs. KLF6-manipulated aged cells were compared to unmanipulated CD34+ cells from both young and aged healthy donors, as well as to aged cells from the same donor but transfected with non-targeting control sgRNA (NTC). Activation with dCas9-VP64 resulted in persistent KLF6 re-expression, even after 9 days in liquid culture, demonstrating that we have developed a robust method for in vitro manipulation of KLF6 levels. Using this system, KLF6 upregulation was induced in young and aged CD34+ cells. Cells with KLF6 re-expression were FACS-sorted and used for in vitro assessment of myeloid differentiation potential in liquid culture. In order to determine whether KLF6 re-expression in aged HSPC induced a revitalization effect in these cells, the myeloid differentiation potential of KLF6-manipulated aged cells was compared to unmanipulated CD34+ cells from both young and aged healthy donors, as well as to aged cells from the same donor but transfected with NTC sgRNA. Unmanipulated aged cells differentiated more efficiently into myeloid cells expressing CD33 (50% by day 7) or CD11b (9.94% by day 7) than their young counterparts (26% and 7.5%, respectively, by day 7), and the same was true for aged cells transfected with NTC sgRNA compared to young cells transfected with this control sgRNA (CD33=53% vs 25%; CD11b: 10% vs 7.2% by day 7). By contrast, aged cells transfected with sgRNAs targeting the KLF6 promoter and achieving reactivation of KLF6 behaved closer to young control cells than they did to aged control cells (CD33=21%; CD11b=7.5%).

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. If aspects of the invention are described as “comprising” a feature, embodiments also are contemplated “consisting of” or “consisting essentially of” the feature.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein. Other than in the operating examples, or where otherwise indicated, all numbers expressing quantities of ingredients or reaction conditions used herein should be understood as modified in all instances by the term “about” as that term would be interpreted by the person skilled in the relevant art.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure. Moreover, any combination of the above-described elements in all possible variations thereof is encompassed by the disclosure unless otherwise indicated herein or otherwise clearly contradicted by context.

### EXAMPLES

### Example 1

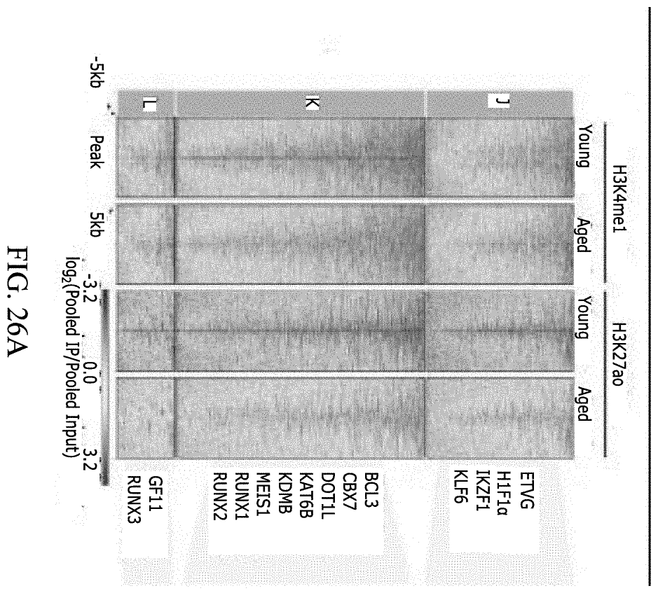

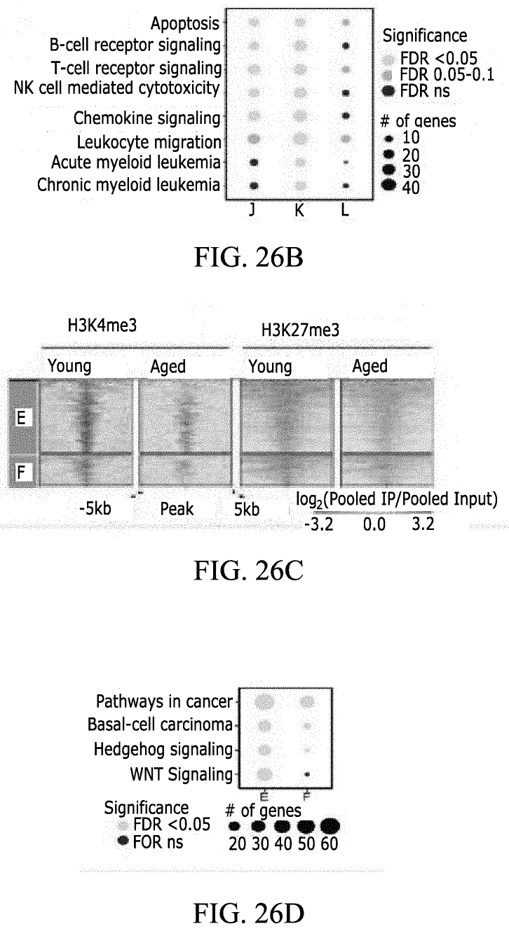

The aging hematopoietic system: As the world population ages, it is imperative that we learn more about the mechanisms underlying age-related dysfunction and pathogenesis. At the hematopoietic level, aging is associated with the development of anemia and other idiopathic cytopenias of unknown significance, loss of adaptive immunity, and an increased risk of myeloid malignancies [1-4]. Additionally, aged hematopoietic stem cells (HSC) have increased self-renewal and expansion with paradoxical dysfunction, resulting in reduced homing ability, accumulation of DNA damage and a myeloid differentiation bias [5-11]. The mechanisms that drive HSC aging are not fully understood. Acquired somatic mutations in the hematopoietic system with aging have been described in healthy individuals, increasing in frequency after the age of 60-70 years, a phenomenon frequently referred to as age-related clonal hematopoiesis (ARCH; also known as clonal hematopoiesis of indeterminate potential or CHIP). The epigenetic modifier genes DNMT3A, TET2 and ASXL1, which are strong myeloid oncogenes, are also the most frequently mutated in ARCH. However, while ARCH is associated with an increased risk of developing hematological malignancies, this risk is very low, at less than 0.5-1% per year [12-15]. This suggests that additional factors must be in play that act cooperatively with these mutations and influence their malignant potential. Epigenetic deregulation may be one such mechanism contributing not only to normal HSC aging dysfunction but also to the observed increased risk of malignant transformation. Recently, we performed the first comprehensive study of epigenetic and transcriptional changes in human lineage—CD34+CD38− cells (HSC enriched fraction; HSCe) with aging. We found that aged HSCe present profound epigenetic reprogramming, with loss of activating histone marks (H3K4me3, H3Kme1 and H3K27ac) which preferentially targets enhancers and bivalent promoters, and gains of hydroxymethylation at introns and exons. These epigenetic changes with aging affect immune cancer related pathways and result in accompanying transcriptional changes [16] (FIG. 26).

Epigenetic deregulation and hematopoiesis: Loss of DNA methyltransferase activity in Dnmt1-deficient murine models is accompanied by a myeloid differentiation bias, suggesting that epigenetic regulation is crucial for maintaining normal homeostasis of the hematopoietic system [18, 19]. Likewise, HSCs from Dnmt3a−/− mice exhibit an inability to silence key stem cell-related genes resulting in aberrant HSC function [20]. Loss of function of either Tet2-involved in the DNA demethylation pathway—or Asxl1—which plays a role in polycomb regulation—in murine models results in development of MDS and MDS/MPN phenotypes [21-24]. In addition, a murine model of Srsf2 P95H mutations that develops MDS demonstrated that this mutation alters Srsf2 by changing its splice recognition function, which results in mis-splicing and degradation of the Polycomb Group 2 gene, Ezh2. Notably, re-expression of wild-type Ezh2 in these cells partially rescued the hematopoietic defect, indicating that epigenetic deregulation contributes to the MDS development in this model [25]. Thus, normal epigenetic regulation is essential for the proper function and development of the hematopoietic system.

Age-related myeloid malignancies: The incidence of acute myeloid leukemia (AML) and myelodysplastic syndromes (MDS) increases with age. While rare before the age of 50, the incidence of MDS increases steadily with every decade of life after that, from 1.5 cases/100,000 people at the age of 50 to 75/100,000 in persons 65 yr., making it twice more frequent among the elderly population than de novo acute leukemia [26], [27]. Despite displaying clinical and morphological heterogeneity as well as a wide spectrum of cytogenetic and molecular abnormalities, we and others have shown that AML and MDS are almost uniformly characterized by marked epigenetic deregulation [28], [29], [30], which in the case of MDS is more profound in patients with more aggressive disease [31]. Notably, epigenetic modifier proteins such as TET2, ASXL1, EZH2 and DNMT3A are frequently mutated in AML, MDS and other age-related myeloid malignancies [32].

KLF6 in hematopoiesis: The KLF family of transcription factors (TFs) comprises 17 members (KLF1-17) with highly conserved DNA-binding domains that regulate an extensive array of essential cellular functions including proliferation, development, differentiation and signal transduction [33-35]. In hematopoiesis, KLF TFs have been implicated in multiple different roles. While it has been shown that KLF2 induces cell growth arrest in lymphoid cells by transcriptional activation of the cell cycle inhibitor p21WAF1/CIP1 [36], data suggest that KLF3 is highly expressed in erythroid cells and that Klf3−/− mice develop myeloproliferative disorders [37]. KLF6, which was the most strongly downregulated member of the family in HSCe with aging in our study [16], is ubiquitously expressed in normal tissues and its expression is lost in many cancers. In solid tumors, KLF6 negatively regulates cell proliferation [38-43]. In acute myeloid leukemia (AML), KLF6 is strongly downregulated and in acute promyelocytic leukemia its levels are restored upon all-trans retinoic acid treatment [44]. Moreover, Klf6 is required for yolk sac hematopoiesis and Klf6−/− mice are embryonic lethal [45]. In addition, and Klf6−/+ mice have increased liver tumor formation and decreased p53 protein levels due to Klf6 transcriptionally repressing the p53-specific E3 ubiquitin ligase Mdm2 [46]. Remarkably, we have seen that in aged HSCe, not only is KLF6 one of the top downregulated genes, but that this correlates with loss of H3K27ac at several upstream putative enhancer regions, indicating that its downregulation may be mediated through epigenetic reprogramming of its active enhancer(s) [16] (see below for more details).

Therefore, as the world population ages, it is imperative that we gain a better understanding of the mechanisms underlying age-related HSC dysfunction and increased risk of transformation in the hematopoietic system. We hypothesize that age acquired epigenetic deregulation at the KLF6 locus in HSC with subsequent loss of KLF6 expression may be implicated in the age-related changes seen in HSC function and may help contribute to the increased risk of malignant transformation. Understanding this mechanism is a necessary step in any plan for the future development of therapeutic interventions with preventive purposes.

Profound age-related epigenetic changes in HSCe target regulatory elements: We performed genome-wide profiling of epigenetic marks, including histone and cytosine modifications, on human HSCe from healthy young (18-30 yr.) and aged (65-75 yr.) donors. Differential enrichment analysis of these cohorts revealed a significant loss of H3K4me1, H3K27ac and H3K4me3, affecting 14,077 (11% of all young peaks), 10,512 (24%) peaks and 18,195 (31%) peaks, respectively (log 10 likelihood ratio >3, absolute fold-change 1.5), with only minimal changes in H3K27me3. Genomic annotation of these changes revealed the loss of 4,124 active enhancers in the aged samples due to loss of H3K27ac, affecting many epigenetic modifier genes (DOT1L, KAT6B, KDM8, CBX7) as well as many key hematopoietic TFs (MEIS1, GFI1, RUNX1, BCL6, RUNX3, and KLF6). Pathway analysis of these lost enhancers showed enrichment for immunity-related signaling pathways, as well as leukemia and apoptosis pathways (FIGS. 26A & B). In addition, 2,171 bivalent promoters displayed a switch from bivalency to repression due to loss of H3K4me3 with aging. Bivalent genes thus affected were associated with key developmental pathways such as WNT and Hedgehog signaling, as well as with cancer-related pathways (FDR <0.05) (FIGS. 26C & D) [16].

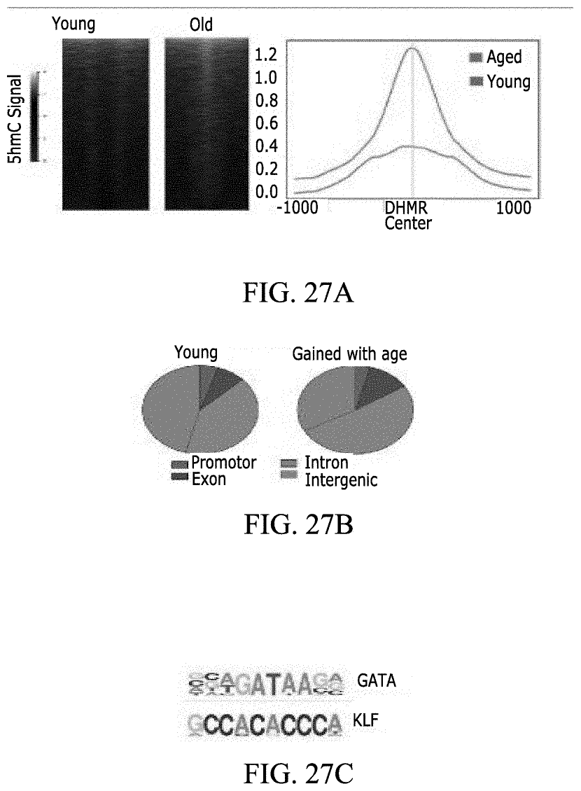

KLF motifs are enriched at areas with age-related changes in 5-hydroxymethylation (5hmC): In addition to changes in histone modifications, aging HSCe also presented with specific DNA methylation (not shown) [16] and 5hmC changes. The latter consisted of gains in 5hmC frequently targeting introns and exons (p-value <2.2e16, binomial test), and were enriched in GATA and KLF transcription factor binding motifs (q value <10-4 for both) (FIG. 27).

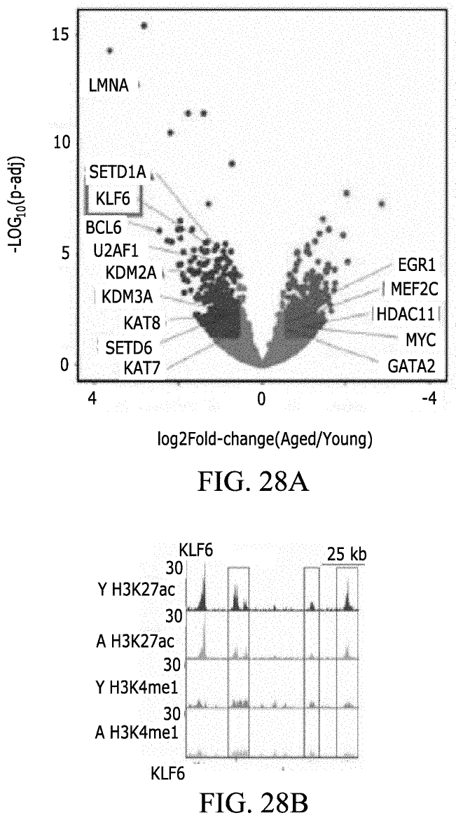

Aged human HSCe have a distinct gene expression signature: Gene expression analysis of young and aged human HSCe revealed an age-specific signature consisting of 1,133 genes (FDR<0.01 and Fold change>1.5), with 517 genes upregulated and 616 downregulated with aging. These included downregulation of hematopoietic TFs such as BCL6, KLF3, KLF6 and KLF7, splicing factors U2AF1 and SREK1, and epigenetic modifiers SETD1A, KDM3A and SETD6. Notably, one of the most downregulated genes in aged HSCe was KLF6. This downregulation correlated with loss of H3K27ac at several putative enhancers, indicating that this downregulation is epigenetically regulated (FIG. 28) [16]. We hypothesize that this age-acquired epigenetic deregulation leading to loss of KLF6 gene expression contributes to the impaired function of aged HSCs.

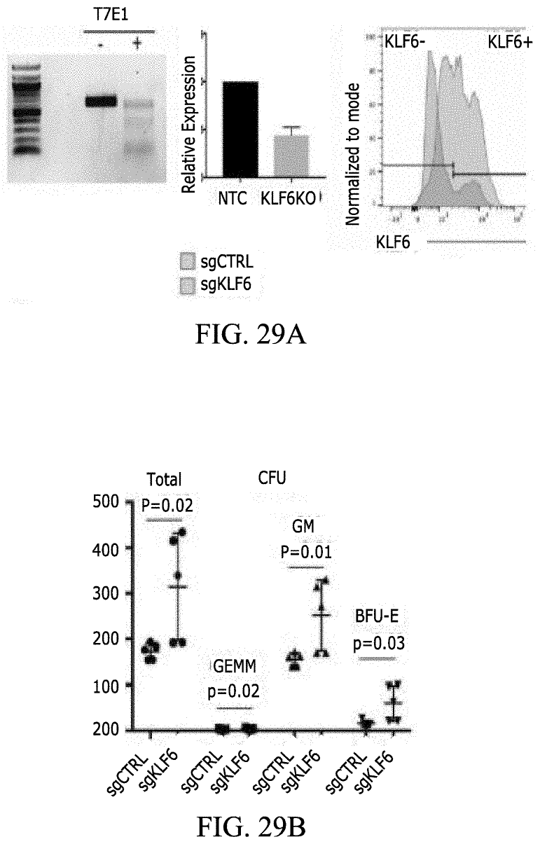

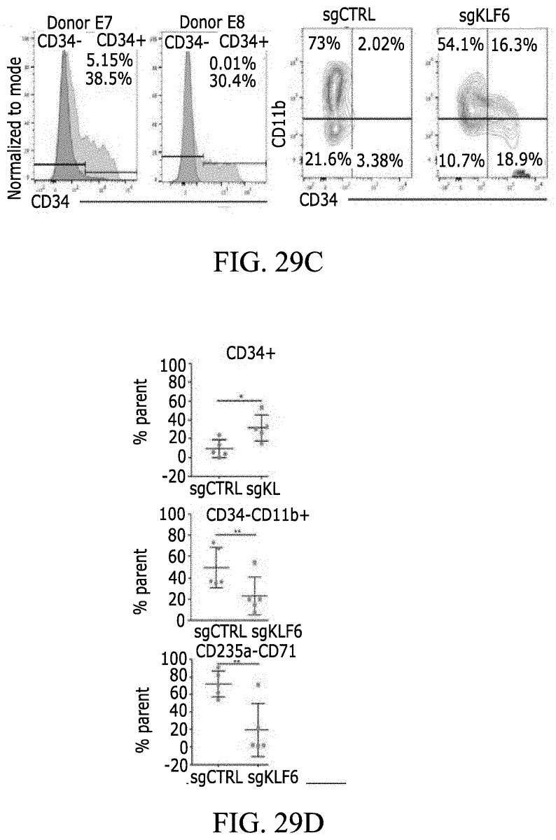

Loss of KLF6 in human HSCe results in an increase in progenitor potential and a differentiation block in vitro: Given the strong enrichment for KFL motifs in the differential hydroxymethylated regions with aging, the strong downregulation of KLF6 in aged HSCe with epigenetic changes at the KLF6 locus, as well as the fact that KLF6 has been shown to be play a role during hematopoiesis development [45] as well as a tumor suppressor [43, 46], we decided to focus on KLF6 amongst all the downregulated KLF transcription factors. Thus, to explore the potential function of KLF6 in human cells, we first established a CRISPR-Cas9 genome editing approach to target the KLF6 gene with specific single guide RNAs (sgRNAs) in primary human CD34+ cells using recombinant Cas9 (rCas9) protein (PNA Bio) [48, 49]. 72 h post-transfection, CD34+ positive cells were enriched by fluorescence-activated cell sorting (FACS) and used to estimate target editing efficiency using the T7 endonuclease 1 (T7EI) mismatch detection assay (FIG. 29A, left). Quantitative RT-PCR (qRT-PCR) of KLF6 mRNA (FIG. 29A, middle) and flow cytometric analysis of KLF6 protein (FIG. 29A, right) showed robust knock-out (KO) efficiency compared to control non-targeting sgRNAs (sgCTRL). KLF6 KO CD34+ cells were plated on methylcellulose to evaluate their colony-forming unit (CFU) potential. KLF6 KO cells exhibited an expansion in the total numbers of colonies with a strong increase in the formation of granulocyte-monocyte colonies (CFU-GM) as well as an increase in erythroid burst-forming units (BFU-E), with minor differences in granulocyte-erythrocyte-macrophage-megakaryocyte (GEMM) (FIG. 29B) indicating that KLF6 plays a role in determining the in vitro progenitor potential of normal CD34+ cells.

Next, in order to evaluate the role of KLF6 in differentiation, we cultured KLF6 KO CD34+ cells under myeloid or erythroid differentiation conditions. KLF6 KO cells were cultured for 3 days in IMDM supplemented with SCF, FLT3, IL-6, and TPO. On day 4, cells were placed under either myeloid-promoting conditions (SCF, FLT-3 ligand, IL-3, IL-6, GM-CSF and G-CSF) or erythroid-promoting conditions (Epo, SCF). For 11 days, cells were evaluated at regular intervals by flow cytometry. KLF6 KO cells displayed signs of differentiation block, with persistence of CD34+ expression even after 7 days in culture (FIG. 29C), accompanied by a reduction in the expression of the CD11b, CD15 and CD33 myeloid markers, and the CD71 and CD235a erythroid markers (FIG. 29D) [16]. Together, these findings indicate that, beyond its role in yolk sac hematopoiesis, KLF6 may also play a role in adult CD34+ cells by determining HSC/progenitor potential and regulating myeloid and erythroid differentiation.

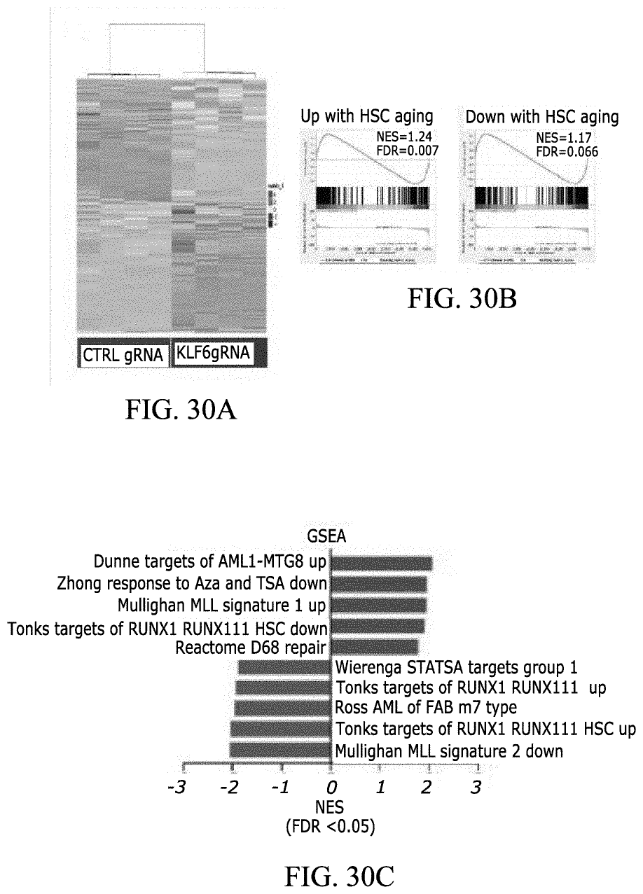

KLF6 knock-out results in gene expression changes that partially recapitulate the aging HSCe signature and are reminiscent of malignant profiles. We sought to define if KLF6 genomic deletion recapitulates, at least in part, the aging HSCe expression profile observed in primary human HSCe (FIG. 28A). For this purpose, we performed RNA-seq analysis after KLF6 KO by genome editing. Differential gene expression between KLF6-K0 cells and control sgRNA identified strong expression changes (FIG. 30A). Notably, gene set enrichment analysis (GSEA) showed that the KLF6 KO expression changes were significantly enriched for our reported HSCe up and downregulated aging signatures [16] (FIG. 30B) as well as for many leukemia and cancer signatures (FIG. 30C) [16]. Thus, downregulation of KLF6, as it occurs during normal aging, may create a permissive environment with many shared features of malignant cells, in which development of age-related myeloid malignancies may be favored.

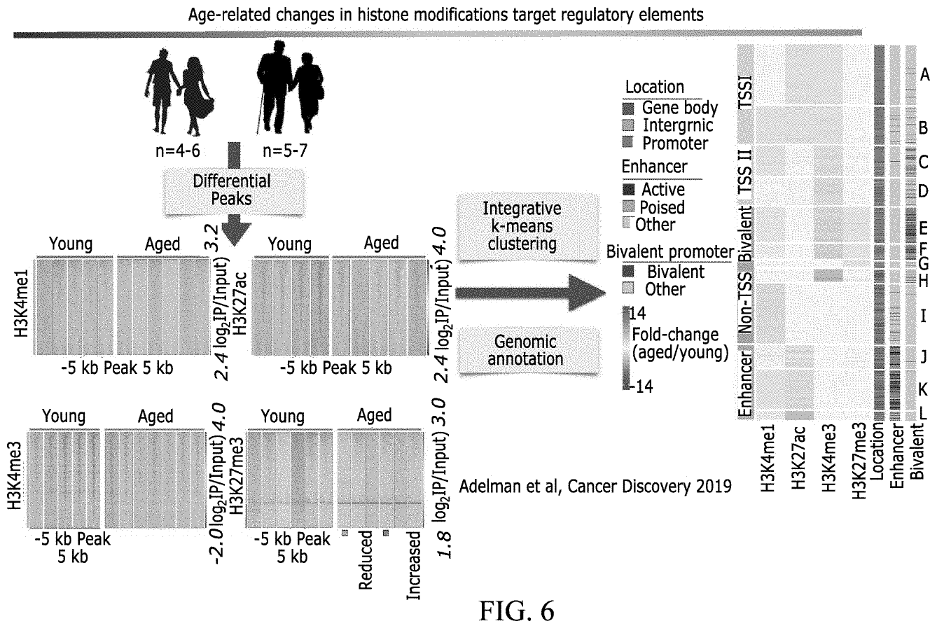

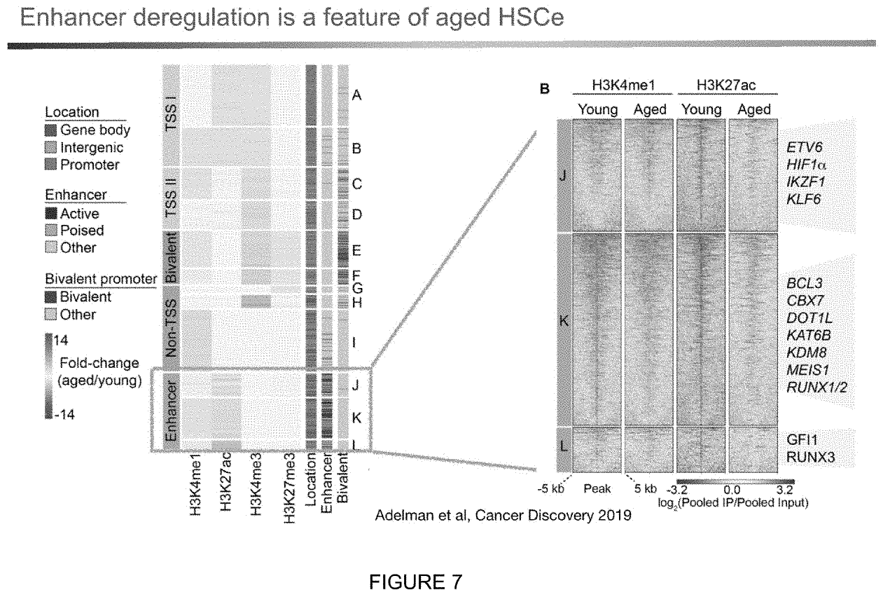

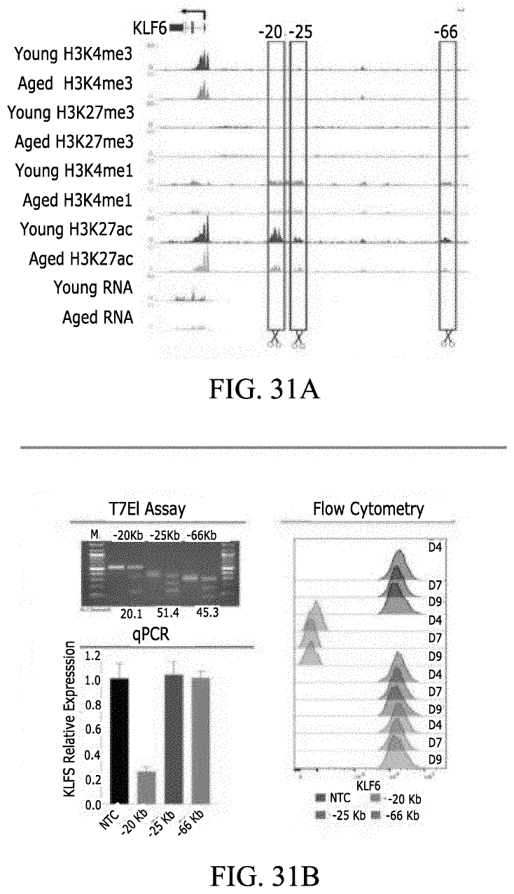

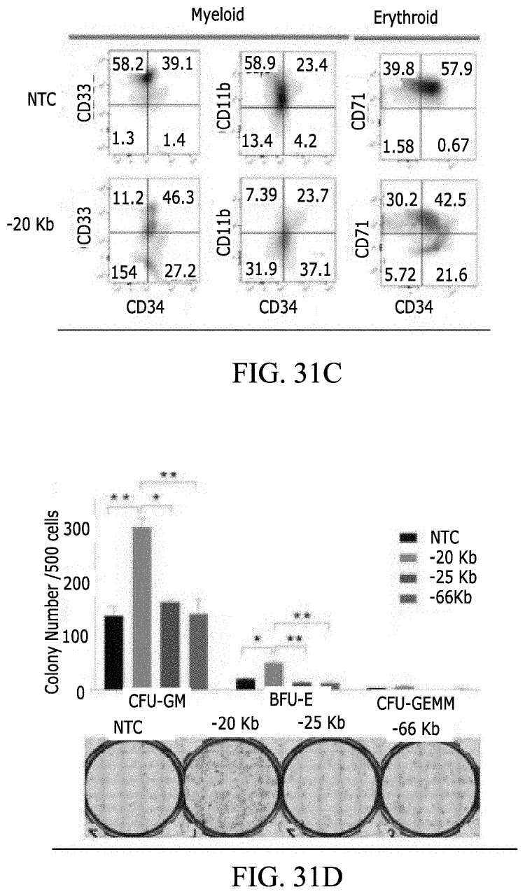

KLF6 expression is regulated by a −20 kb enhancer: Given that multiple putative enhancer regions upstream of the KLF6 promoter showed loss of H3K27ac with aging, we sought to identify which of these elements was responsible for regulating KLF6 expression in adult CD34+ cells. We designed sgRNAs against the closest 3 putative enhancer regions (−20, −25 and −66 kb) upstream of the promoter (FIG. 31A). CRISPR-Cas9-mediated editing of the −20 kb enhancer region, but not the −25 or the −66 kb, led to robust downregulation of the KLF6 mRNA and protein (FIG. 31B). This resulted in a similar block in myeloid and erythroid differentiation in liquid culture as observed after the KLF6 gene KO (FIG. 31C). Moreover, only deletion of the −20 kb region led to enhanced colony formation on methylcellulose (FIG. 6D). RNA-seq analysis after deletion of the −20 kb enhancer region resulted in a gene expression profile with a 79% overlap with the one observed when the KLF6 gene was deleted (p<0.0001). GSEA analysis of this gene profile revealed enrichment for the same leukemia-related gene sets as those enriched after deletion of the gene (data not shown). Therefore, we hypothesize that age-related epigenetic reprogramming with loss of H3K27ac at this −20 kb site is likely responsible for KLF6 downregulation with aging.

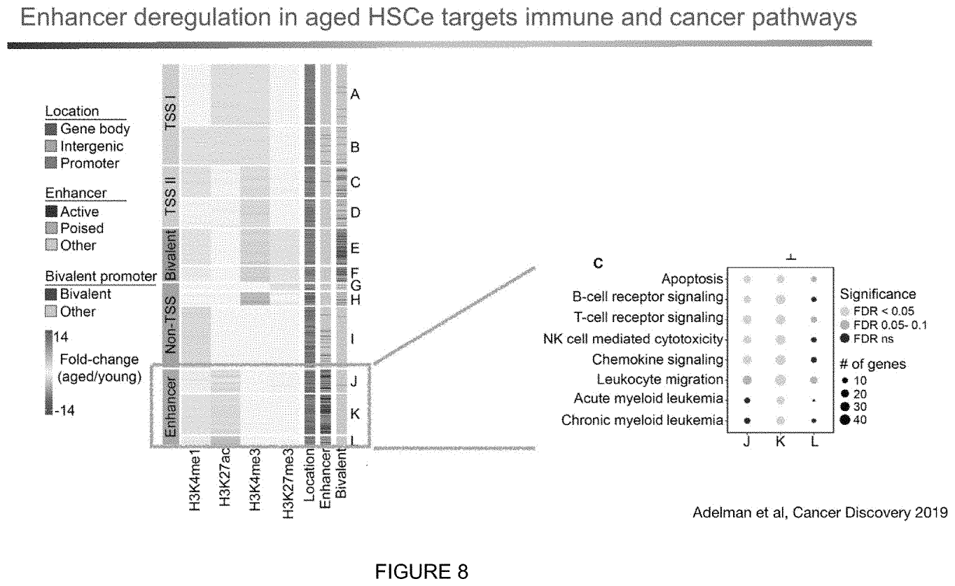

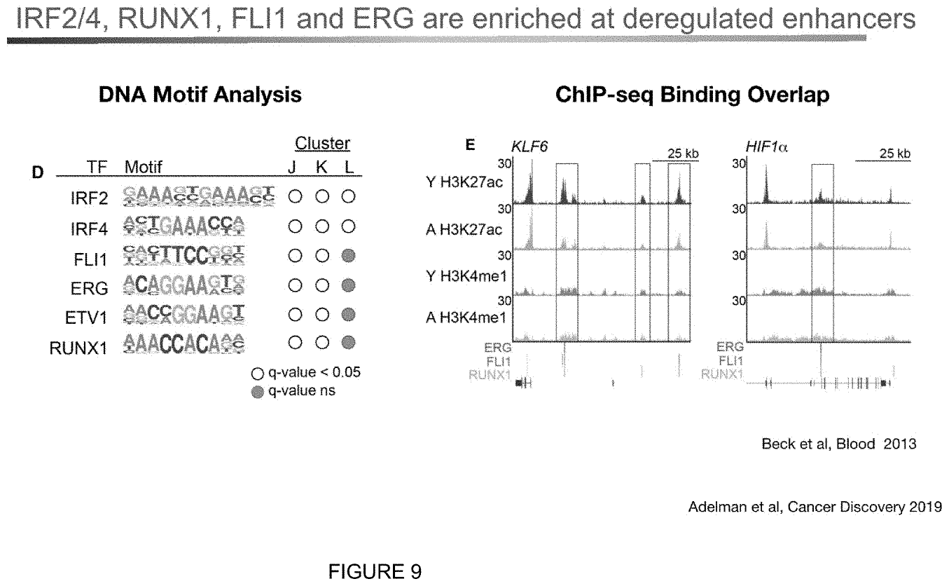

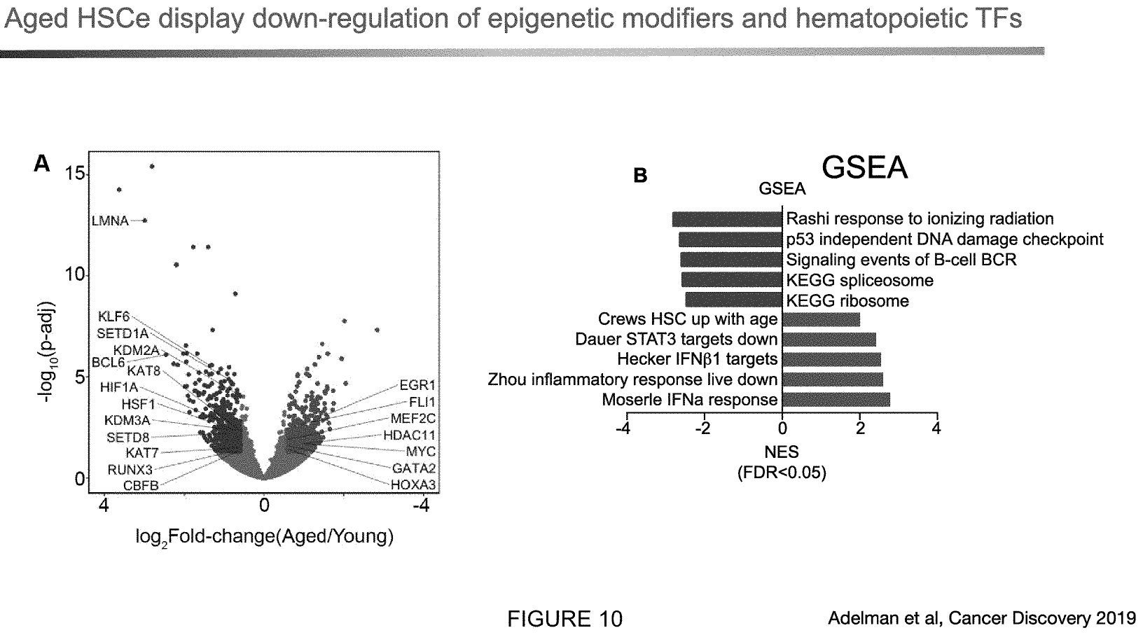

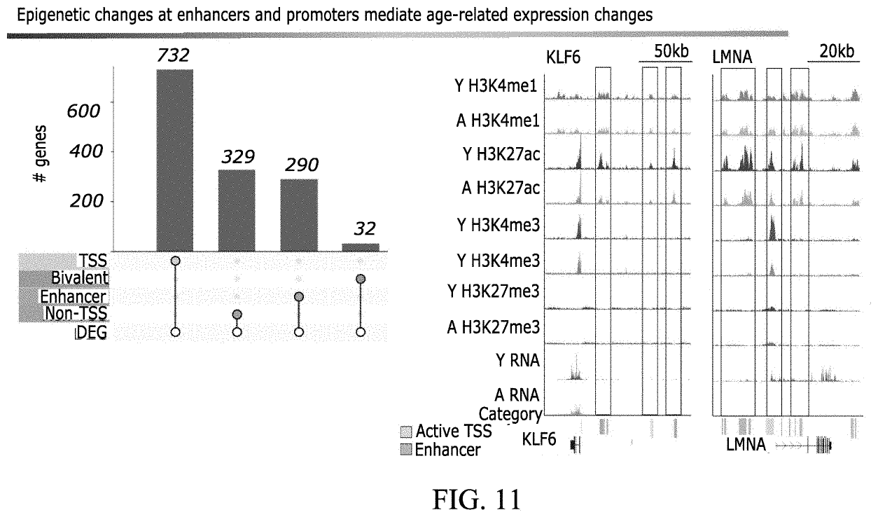

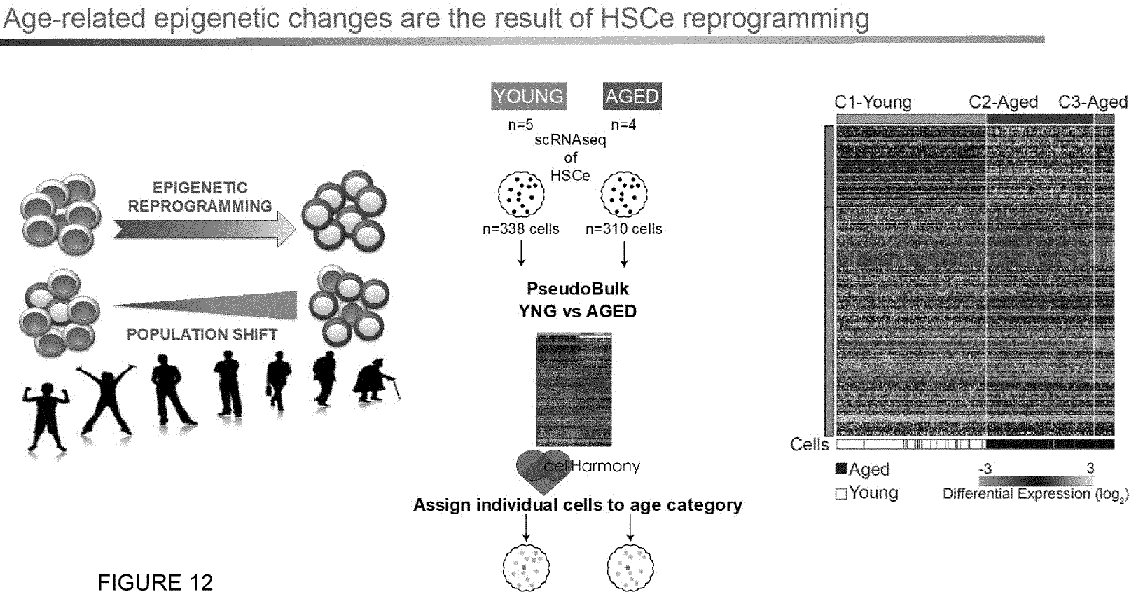

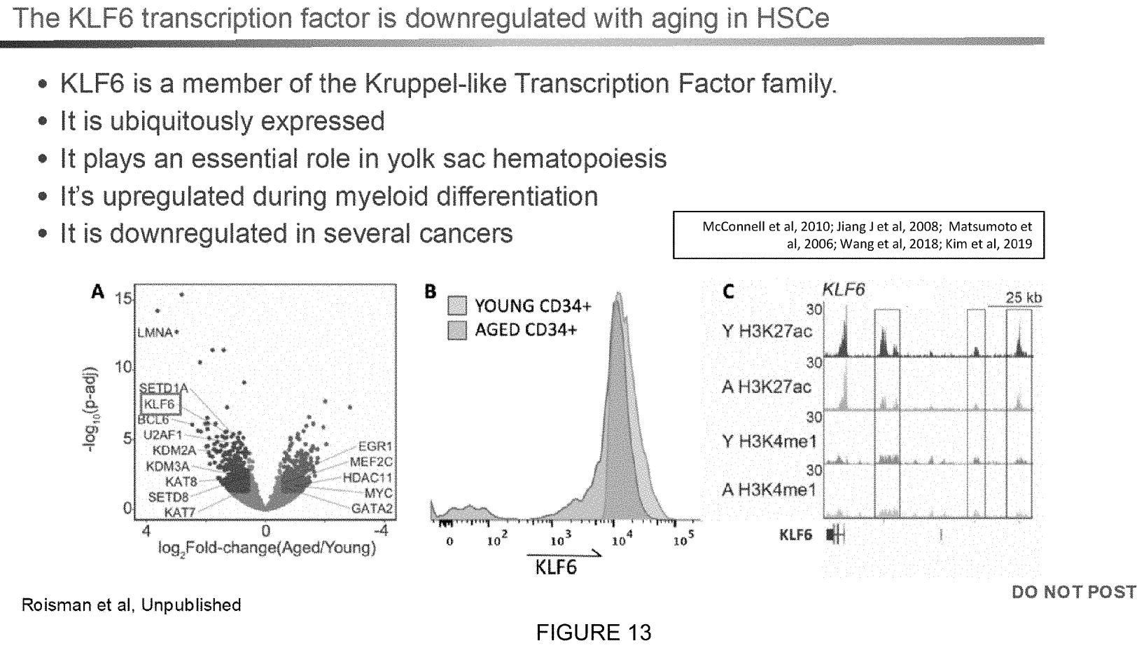

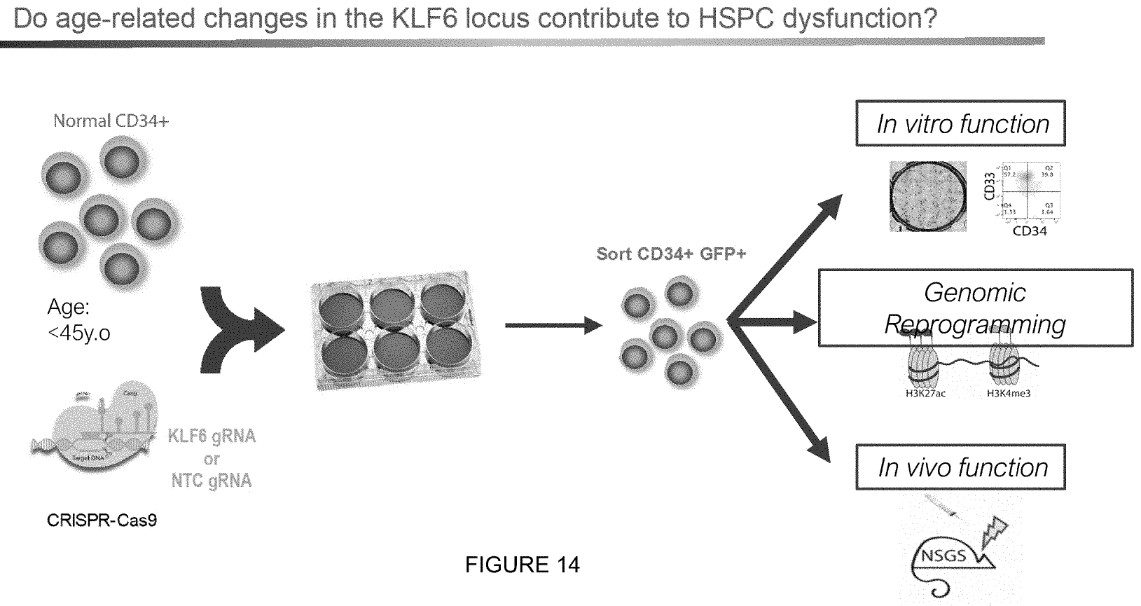

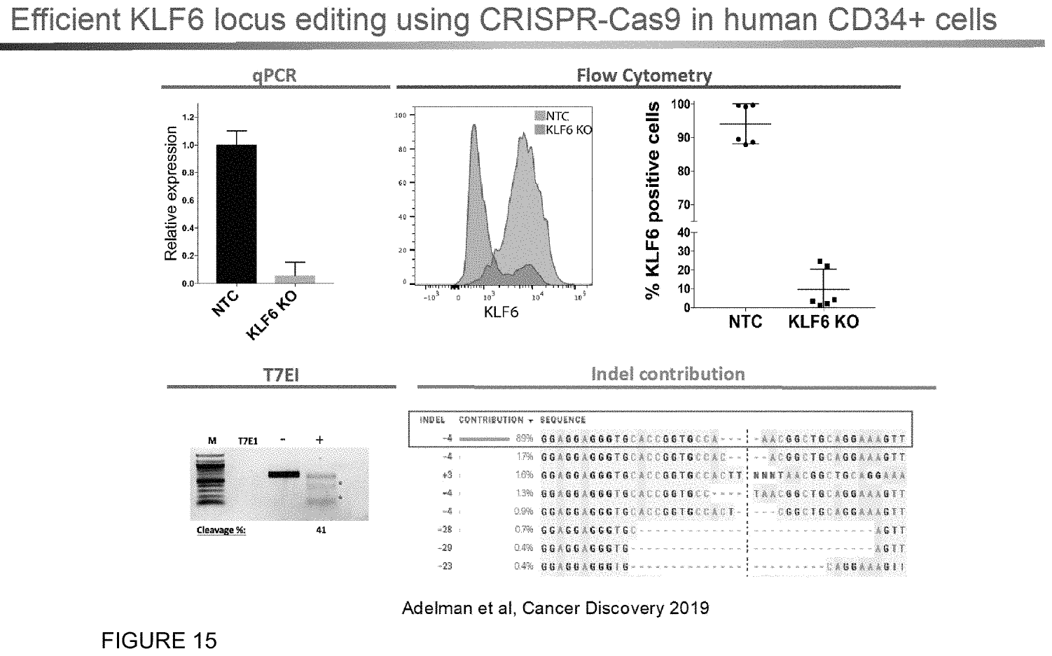

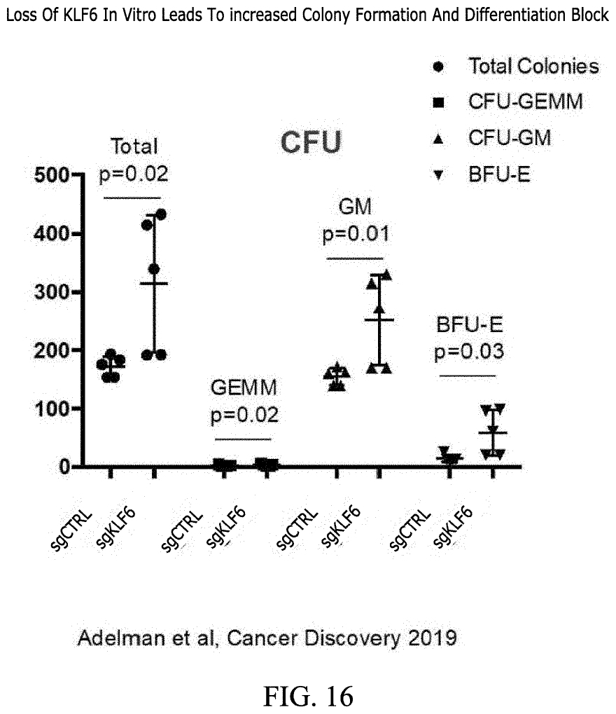

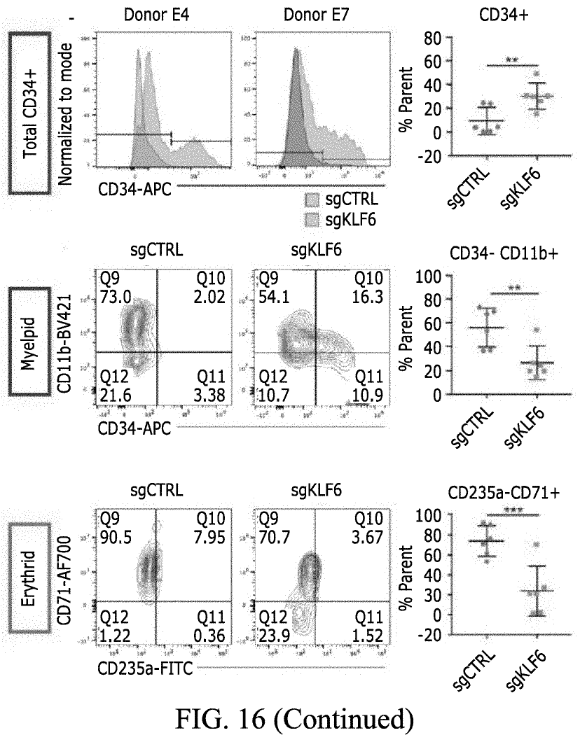

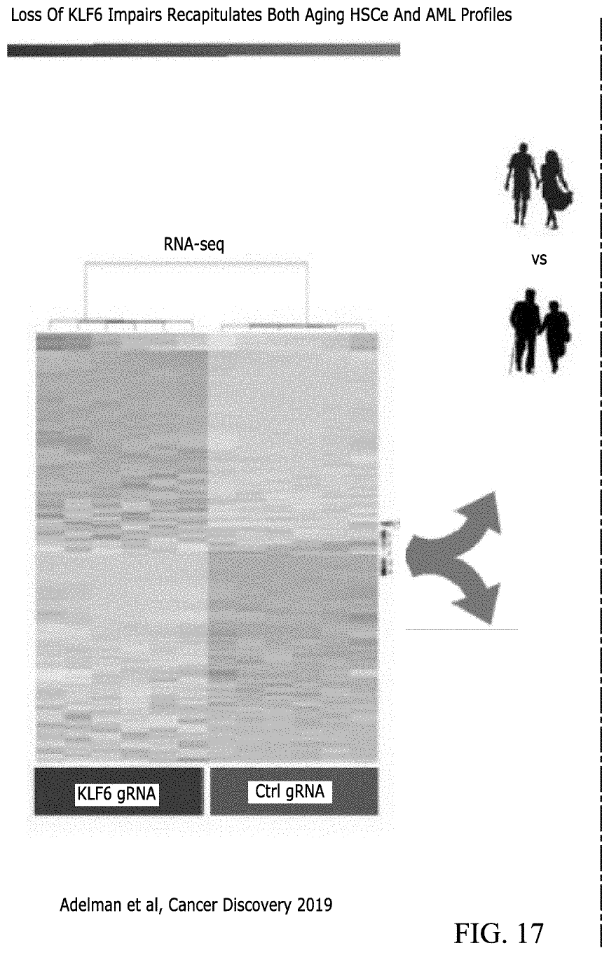

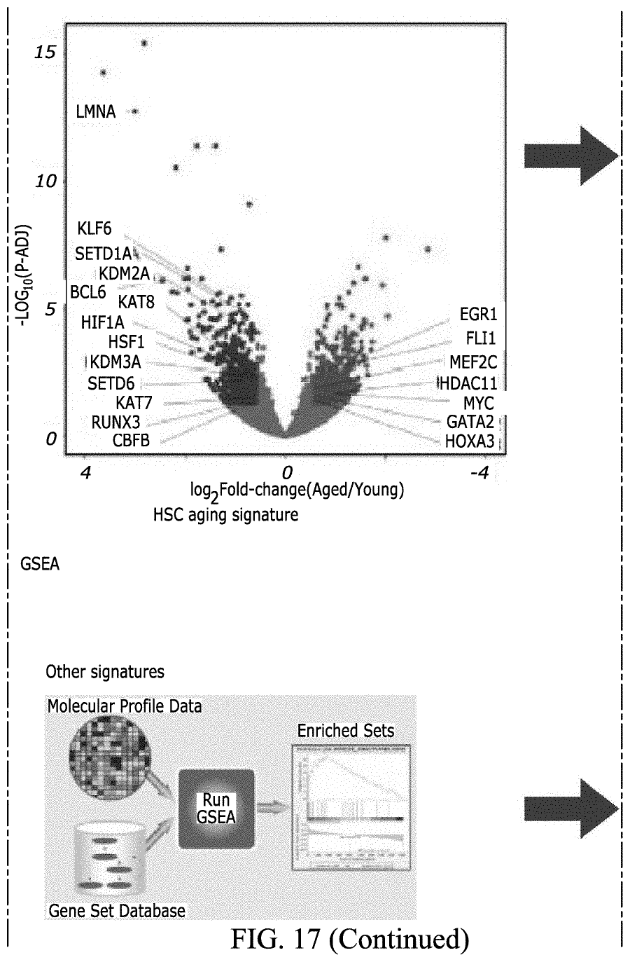

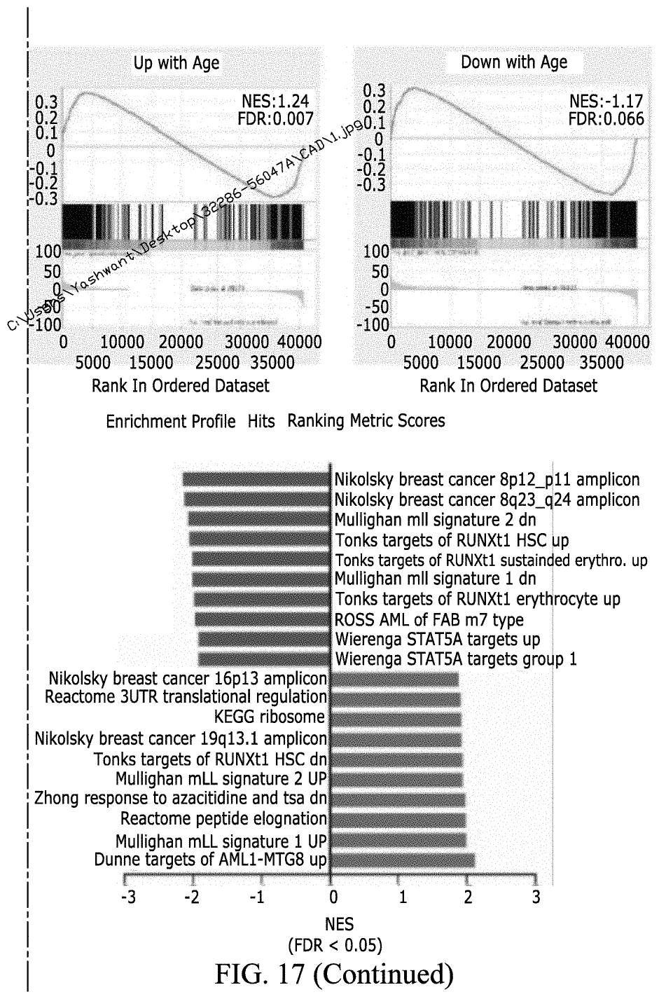

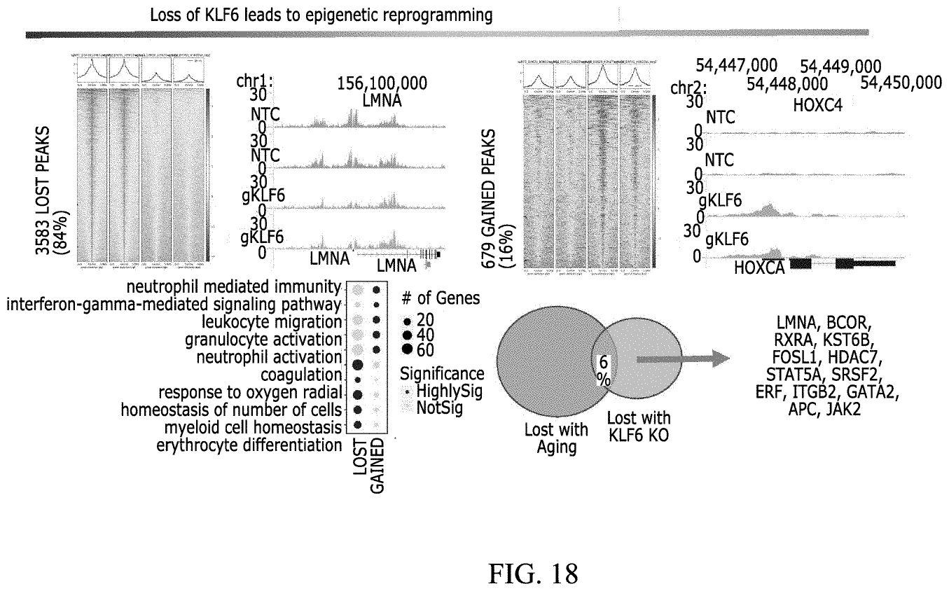

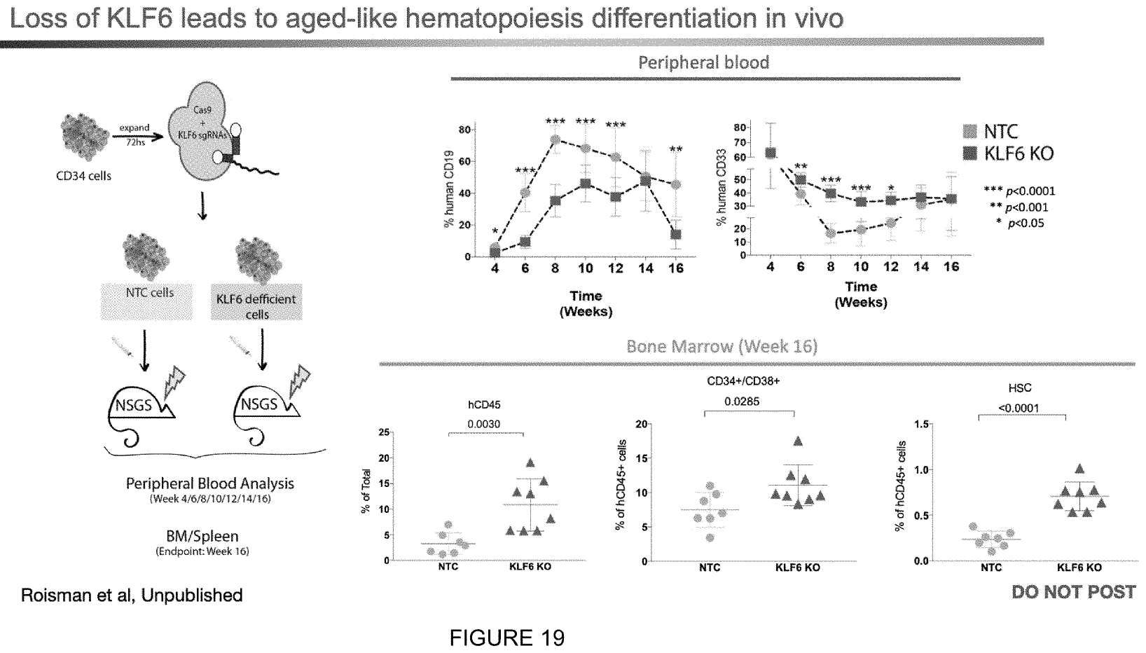

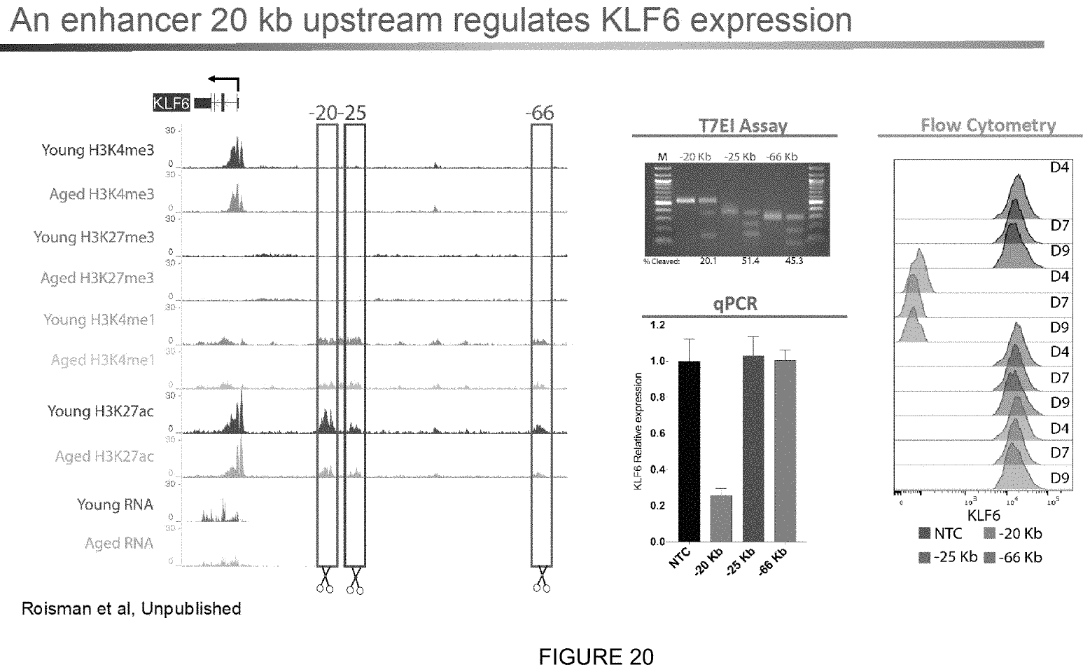

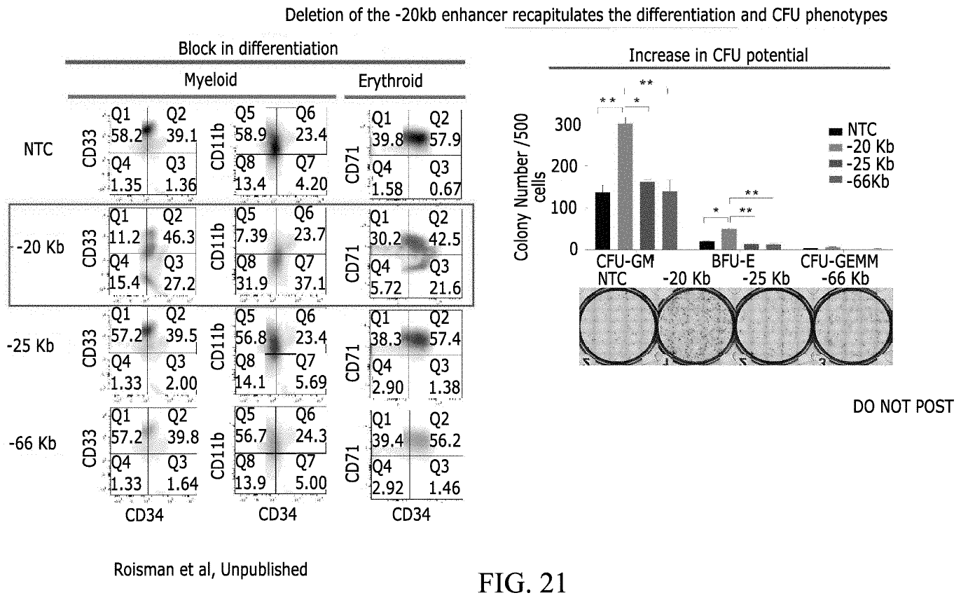

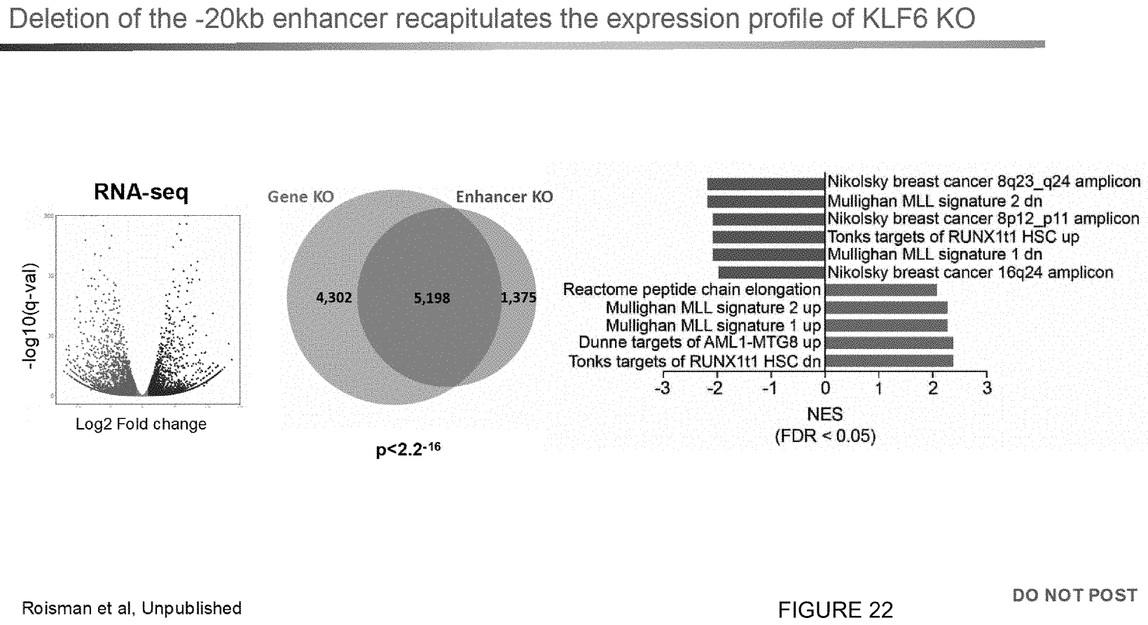

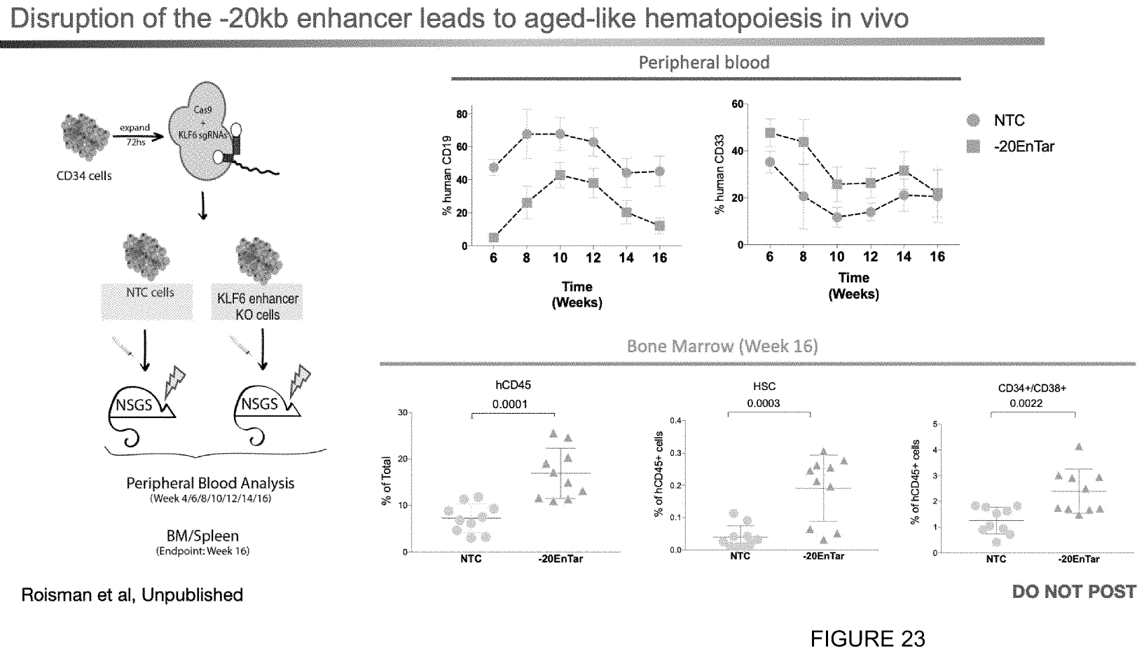

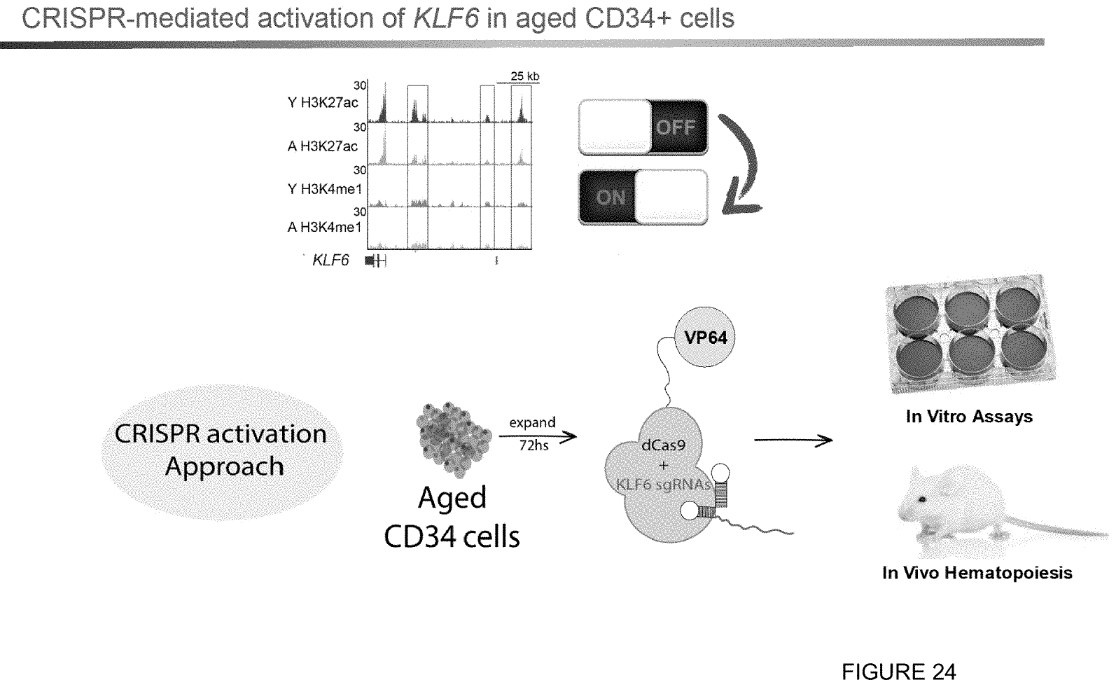

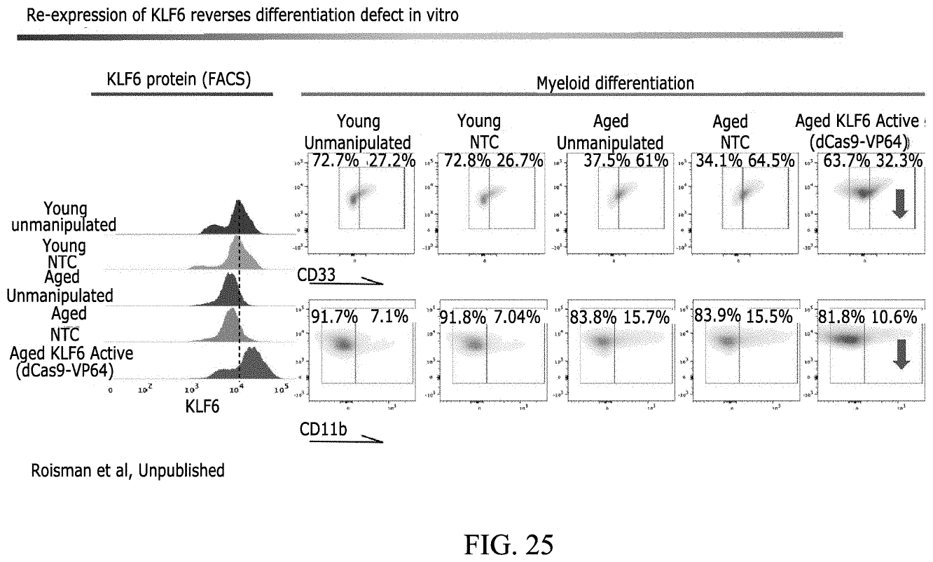

Activation of the KLF6 locus by CRISPRa in age d CD34+ cells leads to an improvement of the myeloid differentiation bias: In order to study the impact of normalizing KLF6 in the context of aging, we established a system based on a catalytically deactivated Cas9 (dCas9) fused to the VP64 transcriptional activator. Lin− CD34+ cells were isolated from the bone marrow of healthy individuals aged either 65-75 yr., obtained from discarded femoral heads after hip replacement surgery, or 18-35 yr. After lentiviral transduction with the pLV hUbC-dCas9 VP64-T2A-GFP (herein, dCas9-VP64; Addgene #53192)[50, 51], the KLF6 promoter was targeted for reactivation through transfection of our validated sgRNAs. KLF6-manipulated cells were compared to unmanipulated CD34+ cells and non-targeting control sgRNA (NTC), as well as across age groups. Activation with dCas9-VP64 resulted in persistent KLF6 re-expression, even after 7 days in liquid culture, demonstrating that we have developed a robust method for in vitro manipulation of KLF6 levels (FIG. 8A). Using this system, we induced KLF6 upregulation in young and aged CD34+ cells. Cells with KLF6 re-expression (or control sgRNA) were FACS-sorted and used for in vitro assessment of their myeloid differentiation potential in liquid culture. Unmanipulated aged cells as well as aged ells transfected with control sgRNA (NTC) differentiated more efficiently into myeloid cells expressing 98.8% CD33 or 41.9% CD11b by day 7, while their young counterparts expressed only 20.3% and 11.8%, respectively). By contrast, aged cells transfected with sgRNAs targeting the KLF6 promoter and achieving reactivation of KLF6 behaved closer to young control cells than they did to aged control cells (CD33=23.6%; CD11b=17%) (n=3 biological replicates) (FIG. 8B). These findings indicate that re-expression of key transcription factors silenced in HSCe during aging, in this case KLF6, can help rejuvenate these cells and partially revert the aging myeloid differentiation bias.

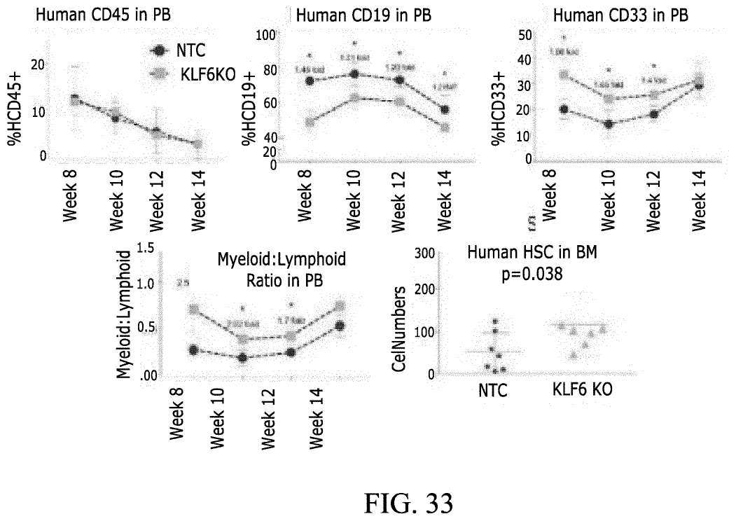

To determine the in vivo consequences of loss of KLF6 or its −20 kb enhancer on human hematopoiesis: In order to expand on our results obtained in vitro, we will next test the impact of KLF6 loss on human hematopoietic reconstitution using an immunodeficient xenograft model. Specifically, we will generate human KLF6-deficient CD34+ cells through CRISPR-Cas9 editing, and compare them to cells with non-targeting control sgRNAs (NTC sgRNA). We will use recombinant Cas9 protein fused to Cy3 from PNA Bio (Cy3-Cas9), in order to be able to fluorescently sort the edited cell population. KLF6-edited cells or their NTC counterparts will be injected into immunodeficient NSG-SGM3 (NOD.Cg-Prkdcscid112rgtm1WjlTg(CMV-IL3,CSF2,KITLG)1Eav/MloySzJ) mice, which allow for better human myelopoiesis and terminal differentiation after CD34+ engraftment than regular NSG mice [52-54]. We have established the protocol in a pilot cohort (n=7 per group) with the goal of establishing feasibility and determining biological variability. This pilot study demonstrated not only robust engraftment of human CD45+ cells after CRISPR-Cas9 genome editing, but also the ability to support human myelopoiesis using NSGS mice. Moreover, this study showed the presence of myeloid bias and HSC expansion in the context of KLF6 KO (FIG. 33), with a trend to GMP and MEP expansion in the bone marrow (data not shown).

Based on our preliminary data, approximately 1.5-2×10⁵ edited cells will be injected per mouse with a total of 8 mice per group for each independent CD34+ donor. Normal CD34+ cells from young (18-35 yr.), healthy donors will be obtained from commercial and academic vendors (AllCells inc., StemCell Technologies, Fred Hutch Cooperative Center of Excellence for Hematology). A total of three independent cohorts, from 3 different donors, will be injected, for a total of 24 mice per group (total=48 mice; 24 NTC and 24 KLF6 KO). This design would give us 90% power at a significance level of 0.05 to detect a true difference in myeloid progenitors in the bone marrow analysis, even in the unlikely situation that one or two mice in either group are lost due to early mortality associated with the procedure itself. Engraftment will be evaluated every 4 weeks, beginning on week 8, through the assessment of human CD45+ percentage by flow cytometry in peripheral blood (PB) of reconstituted mice for up to 16 weeks. Lineage reconstitution analysis will be performed for myeloid (CD33), lymphoid (CD3, CD19), and erythroid (CD71, CD235a) markers in peripheral blood. On week 16, mice will be sacrificed and bone marrow (BM) and spleen cells will be harvested and analyzed for HSC, progenitor, and lineage composition to determine hematopoietic reconstitution potential upon KLF6 KO. Furthermore, since accumulation of DNA damage is a feature of aged HSC [10, 11], we will measure accumulation of DNA damage through the presence and quantification of H2AX foci using an anti-phospho-H2A.X (Ser 139) FITC-labeled antibody (clone: 2F3; 613404, BioLegend) in bone marrow and spleen HSCs. Statistical significance of differences in specific BM cell frequencies between the two groups will be determined using two-way ANOVA to account for the different donors used. Statistical significance for PB engraftment of myeloid and lymphoid lineages over the course of the experiment will be determined using a mixed model for repeat measures data using the nlme package in R (CRAN.R-project.org/package=nlme). Statistical significance in differences in DNA damage between the groups will be determined using a two-sided paired Student's T-test. Next we will repeat the experiment using sgRNAs targeting the −20 kb enhancer region, as in FIG. 6. We hypothesize that loss of the −20 kb region will phenocopy the loss of the KLF6 gene itself, confirming its role in regulating KKLF6 expression and resulting in hematopoiesis that shares features with those seen with aging i.e. myeloid bias, HSC expansion and increased DNA damage. Experimental design as well as sample size and power calculations for this second xenotransplantation are identical to the first one and would therefore also give us would give us 90% power at a significance level of 0.05 to detect a true difference in myeloid progenitors in the bone marrow analysis.

Evaluate whether KLF6 modulation results in epigenomic reprogramming of HSCe: Since aging is a polygenic phenomenon, we don't expect that loss of KLF6 would explain all features of aged HSCe. Therefore, in order to determine how much of the epigenetic reprogramming observed in aged HSCe is due to loss of KLF6 and, more specifically, to identify the biological pathways regulated by KLF6 in these aged cells, we will evaluate changes in the chromatin landscapes of HSCe upon KLF6 knock-out and activation using antibodies against H3K4me1 (Diagenode C15410194 [pAb-194-050]), H3K4me3 (Diagenode C15410003), H3K27me3 (Active Motif 391550) and H3K27ac (Abcam Ab4729). In addition, we will perform chromatin immunoprecipitation (ChIP) for KLF6 using 2 different ChIP-validated antibodies from Santa Cruz Biotechnology, E10 and R-173 [55], which will be further validated by repeating the KLF6 ChIP in the context of KLF6 knock-out. ChIP-seq will be performed for each of those marks on 15,000-25,000 Lin− CD34+CD38+ (HSCe) cells in which the KLF6 locus has been engineered as described above, except for KLF6, in which 50,000-100,000 cells will be used instead. All ChIP experiments will be performed in biological triplicates after KLF6 manipulation. We will use the True MicroChiP kit from Diagenode followed by library preparation using the Swift Biosciences AcceI-NGS 2S Plus DNA Library Kit, a protocol well established in the Figueroa Lab [16]. Libraries will be multiplexed and sequenced on a Next-Seq Illumina sequencer with a target goal of a minimum of 30 million unique reads per library for narrow-peak histone marks and KLF6, and 45 million unique reads for H3K27me3, as per ENCODE 3 guidelines. After quality control using the FastQC pipeline (www.bioinformatics.babraham.ac.uk/projects/fastqc/), reads will be aligned to the reference genome and peak-calling will be performed using MACS2 [56]. Reproducibility across biological replicates will be assessed using the Irreproducible Discovery Rate (IDR) [57]. Differential peak calling between conditions (baseline, KLF6 KO and KLF6 activation) will be performed using DiffBind, which allows for differential peak calling preserving information from the individual replicates [58]. Epigenetic reprogramming upon KLF6 modulation will be compared to the aged HSCe profiles previously reported by our lab [16]. Integrative analysis was performed comparing the histone profiles to KLF6 binding, in order to identify direct KLF6 target regions vs downstream effects. In addition, epigenetic changes obtained upon KLF6 KO in young HSCe cells was directly compared to those from aged HSCe in which KLF6 is re-expressed through CRISPRa. Any changes identified as showing opposite behavior in these two conditions was considered as loci directly regulated by KLF6. Finally, pathway and gene ontology analysis was performed using ChIP-enrich [59] to identify pathway and biological processes specifically regulated by KLF6 in HSCe.

Identify the key transcription factor(s) upstream of KLF6: Our data indicate that KLF6 expression is regulated through a −20 kb enhancer. Both in silico analysis as well as published ChIP-seq data [60] point to GATA2, SMAD1, ERG, RUNX1, and FLI1 as potential candidates for regulating the expression of KLF6 in hematopoietic CD34+ cells. We performed shRNA downregulation (including 5-6 different hairpins per gene) in myeloid cell lines and primary CD34+ cells, and measured protein by western blot analysis and RNA levels by quantitative RT-PCR. In addition, we used myeloid cell lines to introduce point mutations by CRISPR-Cas9 in the DNA binding sites for these TFs at the −20 kb enhancer region and follow protein and RNA levels for KLF6 over a period of 7 days. We expected that modulation of one or more of these TFs will have an impact of KLF6 expression levels. Identification of the key TFs regulating KLF6 allows identification of relevant co-regulators, which would in turn sheds light on potential mechanisms for the modulation of KLF6 expression as a potential intervention for preventing age-related HSC dysfunction.

Expected outcomes: Based on our in vitro preliminary data, we expect that KLF6 will be required for normal hematopoiesis in vivo and that KLF6 knock-out will result in recapitulation of aged-like hematopoiesis, with a myeloid bias and HSC expansion, as seen during aging. We further expect that, like the expression profile, at least part of the age-related epigenetic profiles will be recapitulated upon loss of KLF6. In turn, when using the KLF6 activating approach (CRISPRa) in aged cells we expect to observe, at least in part, a rescue in the epigenetic profiles of aged HSCe. Moreover, we expect to identify the key TF(s) and coregulators responsible for regulating KLF6 expression in HSCs Finally, through our biochemical approaches, we expect to identify the KLF6 interaction partners in hematopoietic cells. These last two steps will prove essential for any future plans to manipulate KLF6 function for preventive and therapeutic purposes.

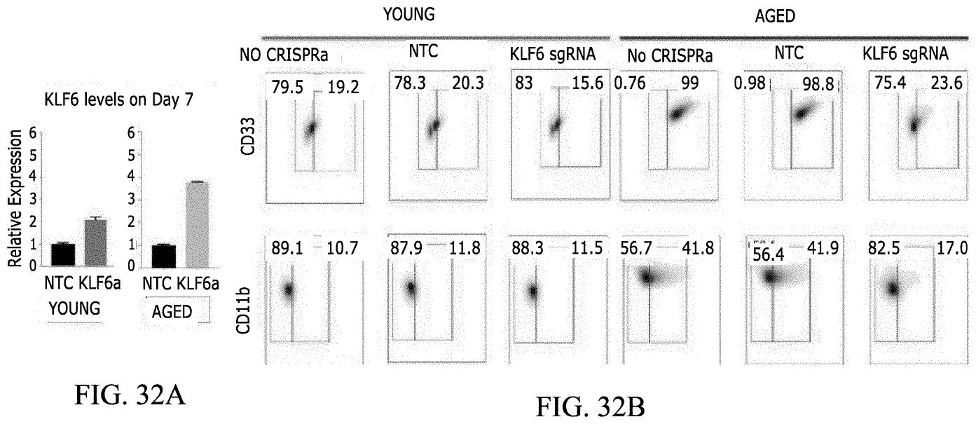

In vitro screen for revitalization factors: From our preliminary data in which we have identified the genes deregulated in aged HSCe compared to their young counterparts, we have prioritized a list of 25 candidate genes for screening in revitalization studies based on their biological and statistical significance. This list contains transcription factors and coactivators (e.g. FOSL2, BCL6, BCL3, KLF7, KLF10, KLF2, HIF1A, RARA, REL, RELB, JUND, MXD4, RUNX3, CBFB, HSF1, TAF9B), epigenetic regulators (e.g. KDMSD, SETD1A, SETD8, KDM3A, SETD6, BRD7, BRD9, KDM2A), regulators of cytokine signaling (e.g. SOCS3, SOCS4), regulators of ubiquitination (e.g. FBXO7, UBE2D2, FBXO11) and LMNA, a structural protein of the nuclear lamina that helps regulate chromatin structure through its role in lamin-associated domain formation and which is frequently mutated in Hutchinson-Gilford progeria syndrome. While we have prioritized TFs in this list, we hypothesize that concomitant downregulation of epigenetic modifier proteins and/or key regulators of signaling may compound the age-related defect in TFs and have therefore included the top scoring candidates in these categories as well, since they may indeed act cooperatively with key TFs to regulate the phenotype. We will first score each of these genes individually for its ability to revert the aging phenotype in a myeloid in vitro differentiation assay using CRISPRa driven by dCas9-VP64 as in our preliminary data (FIG. 32) and identify those that show at least partial reversal of the aging myeloid differentiation bias. While not the only feature of aging, myeloid differentiation bias was chosen as our screening platform for its low complexity, amenability to large-throughput screening, and relatively low cost. sgRNAs will be designed as before, using the Zhang lab tool. Briefly, CD34+ cells will be transduced with the dCas9-VP64 lentiviral plasmid followed by transfection with the relevant sgRNAs against the corresponding target promoter of the test gene or NTC sgRNA. Cells will then be placed in liquid culture under myeloid differentiation conditions with SCF, FLT-3 ligand, IL-3, IL-6, GM-CSF and G-CSF, as before. Cells will be evaluated by flow cytometry on days 4, 7 and 9 for expression of the myeloid differentiation markers CD33 and CD11b, as well as for CD34 expression, as a marker of immaturity. Once again, CD34+ cells from both young and aged healthy donors will be isolated and used in this experiment. We will compare CRISPRa of our selected target genes to (i) NTC gRNAs and (ii) unmanipulated control cells, from both age groups. Young cells (18-35 yr.) will be purchased from academic and commercial vendors as described before. Aged cells (60-75 yr.) will be isolated from banked MNC obtained from discarded femoral heads from hip replacement surgeries performed on individuals with no known hematologic or chronic inflammatory disorder. Over 100 aged MNC specimens are available in the bank, with an average age of 62.3 yr, 43% of them from female donors and 57% from male donors. A positive hit on the screen will be considered when activation of a specific gene acts to induce a partial or complete reversal of the myeloid differentiation phenotype seen in aged cells.

Identification of the best performing gene combination in inducing revitalization of HSPCs: After the initial list of genes has been narrowed down based on positive hits in our in vitro myeloid differentiation screen, we will then test positive hits in combinatorial approaches with the goal of identifying the smallest and most robust combination that achieves revitalization of HSPCs. For this purpose, we will use the multiplex CRISPR-dCas9 system developed by the Gersbach lab [51] and we will systematically test different combinations of the positive hits in our myeloid differentiation assay, as above. Once the strongest combination has been selected, we will then validate the role of each component by sequentially removing one at a time form the combination and repeating the functional experiments. Any gene whose removal from the combination does not have an impact on the revitalizing phenotype, will be removed from the combination. Finally, once the strongest combination has been identified and validated, we will repeat the combinatorial gene activation in CD34+ cells from aged donors followed by Lin− CD34+CD38− HSCe isolation by FACS. We will use these to perform additional assays of aging. RNA-seq, which will be performed in 5 independent biological replicates and we will compare the expression profile of cells activated with the best-performing gene combination to their matching NTC. Changes in gene expression upon CRISPRa activation of the gene combination will be compared to the original signature of aged human HSCe that we have previously reported (FIG. 28) with the goal of determining whether activation of this gene combination results in partial or even complete reversal of the gene expression profile of aged HSCe. Finally, we will also perform colony-forming assays to assess progenitor potential.

In vivo validation of the revitalization potential of the best performing gene combination: Finally, in order to validate the effect that re-expression of the identified gene combination has on in vivo hematopoiesis of aged specimens, we will perform cooperative gene activation (based on the panel of genes identified in point ii above) in aged CD34+ and transplant those cells into NSGS recipients. Hematopoiesis output, including both peripheral blood myeloid and lymphoid engraftment for 20 weeks as well as bone marrow subpopulation composition on week 20, will be compared to NTC in aged CD34+ and NTC in young CD34+. Once again, we will use a total of 8 mice per group for each independent CD34+ donor and a total of three independent cohorts, one from each donor, will be injected, for a total of 24 mice per group. This design would give us 90% power at a significance level of 0.05 to detect a true difference in myeloid progenitors in the bone marrow analysis, even in the unlikely situation that one or two mice in either group are lost due to early mortality associated with the procedure itself. We expect that cells subjected to cooperative gene activation will show a hematopoiesis output close to that observed in the young cells with NTC. Statistical analysis will be performed as in SA1.i.

Expected outcomes: We expect to identify 2 or more genes whose combined re-expression in aged human HSPCs and HSCe leads to the partial or complete reversal of the myeloid differentiation phenotype and the expression profile, as well as to a normalization of in vivo human hematopoiesis in an NSGS xenograft model.

### Example 2

Precise and accurate regulation of hematopoietic stem cell (HSC) function is fundamental in order to maintain the normal production of all blood lineages throughout an organism's lifespan. Aging is associated with a continuous and progressive impairment of HSC function. Furthermore, aging is one of the strongest risk factors for the development of hematological malignancies [1-4]. Aged HSCs are characterized by impairment in self-renewal and homing capabilities, decreased lymphoid differentiation potential with a consequent myeloid bias, and an increased risk of malignant transformation [5-8]. This functional impairment is due in part to cell intrinsic changes such as the presence of accumulated DNA damage [9-13], an increase in reactive oxygen species (ROS) and oxidative phosphorylation [14, 15], a switch from canonical to non-canonical WNT signaling [16], impaired autophagy [17], and the presence of gene expression and splicing changes even in the absence of splicing factor mutations [18], as well as extrinsic cues from an aged and inflammatory microenvironment [19, 20]

We have recently demonstrated that human lineage negative CD34+CD38− cells (HSC enriched; HSCe) exhibit extensive epigenetic reprogramming with aging, characterized by the loss of active enhancers regulating key hematopoietic transcription factors and pathways. This age-associated rewiring of HSCe affects the transcriptional profiles of many epigenetic regulator genes and transcription factors, including Krüppel-like factor 6 (KLF6), one of the most downregulated genes in this context [21]. Kruppel-like factors (KLFs) are evolutionary conserved DNA-binding transcriptional regulators that perform diverse roles during proliferation, development, differentiation and signal transduction [22-24]. Deregulation of these transcription factors has been linked to the pathobiology of numerous diseases, including cardiovascular disease, metabolic disorders and cancer [25-33]. Some members of the KLF family, in particular KLF4, KLF2, and KLF5, have been extensively studied for their involvement in reprogramming differentiated somatic cells to embryonic stem (ES) cells and maintaining self-renewal. It has been postulated that other members of the KLF family may play similar roles in stem cells [22]. Amongst the KLF family of transcription factors, KLF6 has been shown to be strongly downregulated in both human and mouse HSCs with aging [21, 34]. Klf6 null mice are embryonic lethal due to defective hematopoiesis and yolk sac vascularization, and ectopic overexpression of Klf6 enhances the hematopoietic potential of embryonic stem cells [35]. In addition, Klf6 is essential for vascular niche formation and hematopoietic stem and progenitor cell (HSPC) development in the zebrafish caudal hematopoietic tissue [36]. Having previously reported that KLF6 becomes aberrantly silenced during normal human HSCe aging, and that loss of KLF6 leads to an impairment of in vitro differentiation and enhanced colony-forming potential [21], we sought to further investigate the mechanisms behind this deregulation in human cells and its consequences for normal hematopoietic aging.

Methods

Human bone marrow samples and CD34 isolation: Aged (>65 yo) samples were derived from femurs from individuals undergoing hip replacement surgery at the University of Miami Miller School of Medicine. Donors had no known history of hematological malignancy. Bone marrow mononuclear cells (BM MNC) were isolated using ficoll based density gradient centrifugation, enriched using CD34 MicroBeads (Miltenyi, San Diego, Calif., cat. 130-100-453) on an Automacs Pro separator (Miltenyi, San Diego, Calif.) and cryopreserved before further use. Young human CD34+ hematopoietic stem and progenitor cells (<32 yo) from mobilized peripheral blood of anonymized healthy donors were obtained from Fred Hutchinson Cancer Research Center, Seattle, Wash.

Cell culture: Human CD34+ hematopoietic stem and progenitor cells from mobilized peripheral blood (PB) were thawed via dropwise addition of stem-cell-promoting media Iscove's Modified Dulbecco's Medium (IMDM) (Thermo Fisher Scientific), 20% human serum AB (Gemini Bio-Products, cat. 100-512) supplemented with human stem cell factor (SCF) (100 ng/ml), human thrombopoietin (TPO) (Peprotech, cat. 300-018), (100 ng/ml), recombinant human Flt3-ligand (Flt3-L) (Peprotech, cat. 300-019), (10 ng/ml), human interleukin 6 (hIL-6) (Peprotech, cat. 200-06), (20 ng/ml), 1% non-essential amino acids supplement (Thermo Fisher Scientific, cat. 11140050), 1% GlutaMAX Supplement (Thermo Fisher Scientific, cat: 35050061) and 1% penicillin/streptomycin (Gibco™, cat. 15140122). BM or PB mononuclear cells were thawed via dropwise addition of stem-cell-promoting media IMDM (Thermo Fisher Scientific), 20% Human Serum AB (Gemini Bio-Products, cat. 100-512) and DNaseI. Final concentration of DNase I (Roche Applied Science, 10104159001) in IMDM solution was 200 ug/mL. Post-thaw, cells were spun at low RPM (˜1200) for 5 min at 4° C. After the spin, thawing solution was removed and cells were resuspended in stem-cell-promoting media under low oxygen conditions to maximize maintenance and expansion of LT-HSCs [74-76]. Human umbilical cord blood-derived erythroid progenitor (HUDEP) clone 2 (HUDEP-2) cell line was used as previously defined [77]. Briefly, HUDEP-2 cells were expanded in StemSpan SFEM (Stem Cell Technologies, cat 0965) supplemented with 10-6M dexamethasone (Sigma, cat. D4902-25MG), 50 ng/ml human stem cell factor (SCF) (Peprotech, cat. 300-07), 3 international units (IU) IU/ml erythropoietin (EPO) (Peprotech, cat. 100-64), 1% GlutaMAX Supplement (Thermo Fisher Scientific, cat: 35050061) and 1% penicillin/streptomycin (Gibco™, cat. 15140122). 1 pg/ml doxycycline (Sigma Aldrich, cat D9891-1G) was incorporated in the culture to stimulate expression of the human papilloma virus type 16 E6/E7 genes [41]. Leukemia cell lines were cultured as follows: K562 and MV4-11 cells were cultured in IMDM supplemented with 10% FBS and 1% penicillin/streptomycin (Gibco™, cat. 15140122); HL-60 and KG-1 cells were cultured in IMDM supplemented with 20% FBS and 1% penicillin/streptomycin (Gibco™, cat. 15140122); U937 and THP1 cells were cultured in Roswell Park Memorial Institute (RPMI) 1640 Medium (RPMI-1640) (Gibco™, cat. 11875-093) supplemented with 10% FBS and 1% penicillin/streptomycin (Gibco™, cat. 15140122).

sgRNA synthesis and ribonucleoprotein electroporation: KLF6 guide RNAs were produced as described before [21]. Briefly, protospacer sequences were identified using the CRISPRdesign algorithm (crispr.mit.edu) or CRISPRScan (www.crisprscan.org) (Supplementary Table S10) [78]. DNA templates for sgRNAs contain a T7 promoter, the protospacer sequence, and the sgRNA scaffold sequence [79]. They were produced by PCR using custom forward primers and a reverse primer that amplifies the sgRNA scaffold of the plasmid pKLV-U6gRNA-PGKpuro2ABFP (Addgene #62348). PCR products were purified and in vitro transcribed with the HiScribe T7 High Yield RNA Synthesis Kit (NEB #E2040S) following manufacturer instructions. In vitro transcribed sgRNA products were purified using RNA Clean & Concentrator kit (Zymo Research #R1015). −20 Kb, −25 Kb and −66 Kb modified synthetic sgRNAs (2′-O-methyl-3′-phosphorothioate modifications in the first and last three nucleotides) were from Synthego.

After 48-72 hs in culture, HSPC were electroporated with Cy3-Cas9 protein (PNA Bio, CP06) and sgRNAs using the Neon Transfection System (Thermo Fisher). Transfection conditions were 1600 volts, 10 milliseconds, and 3 pulses. After transfection, HSPCs were cultured in stem-cell-promoting media Iscove's Modified Dulbecco's Medium (IMDM) (Thermo Fisher Scientific), 20% human serum AB (Gemini Bio-Products, cat. 100-512) supplemented with human stem cell factor (SCF) (100 ng/ml), human thrombopoietin (TPO) (Peprotech, cat. 300-018), (100 ng/ml), recombinant human Flt3-ligand (Flt3-L) (Peprotech, cat. 300-019), (10 ng/ml), human interleukin 6 (hIL-6) (Peprotech, cat. 200-06), (20 ng/ml), 1% non-essential amino acids supplement (Thermo Fisher Scientific, cat. 11140050) and 1% GlutaMAX Supplement (Thermo Fisher Scientific, cat: 35050061) before sorting for the Cy3-Cas9+/CD34+ population. After sorting cells were maintained for 72 hs under low oxygen conditions before performing the functional assays.

Transient CRISPR activation sgRNA design and preparation of expression construct: sgRNAs were designed using the SAM sgRNA design tool (sam. genome-engineering.org/database/). Guides were designed to target the first 200 bp upstream of KLF6 TSS and minimal overlap of the target sequence (Supplementary Table S10). sgRNAs were scored according to predicted off-target matches and sgRNAs with the best off-target scores were selected. gRNA expression constructs were produced by PCR using forward primers and a reverse primer that amplifies the sgRNA expression cassette of the plasmid pKLV-U6gRNA-PGKpuro2ABFP (Addgene #62348) [80]. Expression constructs were amplified by PCR using the following primers: forward: GAGGGCCTATTTCCCATGATTCC (SEQ ID NO: 9) and reverse: AAAAAAAGCACCGACTCGGTGCCACTTTTTCAAGTTGATAACGGACTAGCCTTATTTTAACTTGCTATTTCTAGCTCTAAAAcN NNNNNNNNNNNNNNNNNNCCGGTGTTTCGTCCTTTCCACAAG (SEQ ID NO: 8). PCR was performed using Faststart HF Enzyme (Sigma cat. 3553400001) with the following thermal profile: 95 C for 2 min; 30 cycles of 95 C or 20 s, 66 C 20 s, 72 C for 8 s; 72 C for 3 min. PCR constructs were purified using the QIAquick PCR (Qiagen, cat. 28104) purification kit according to the manufacturer's directions and finally eluted in 35 μl of water. Correct size of the PCR constructs was confirmed by electrophoresis separated by gel separation. The amplified sgRNA expression cassettes were verified with Nanodrop to confirm good DNA quality and stored at −20 C.

Stable CRISPR activation sgRNA design: In order to clone the guide target sequence into the lenti sgRNA(MS2)_zeo backbone (addgene #61427) [46] two standard de-salted oligos were synthesized. Fw: CACCGNNNNNNNNNNNNNNNNNNNN (SEQ ID NO: 10) and oligo 2: AAACNNNNNNNNNNNNNNNNNNNNC (SEQ ID NO: 11). Oligos were annealed with T4 PNK (New England Biolabs, cat. M0201L) with the following thermal profile: 37° C. for 30 min, 95° C. for 5 min and ramping to 25° C. at 5° C./min. Annealed oligos were diluted 1:10 and ligated into the lenti sgRNA(MS2)_zeo backbone (addgene #61427) with BsmBI (New England Biolabs, cat. M0201L) and T7 ligase (New England Biolabs, cat. M0201L) with the following thermal profile: 37 C for 5 min and 20 C for 5 min for a total of 15 cycles. Finally, the 2-4 ul of the final reaction were transformed into Stb13 competent cells and plated into Ampicilin plates (Teknova, cat. L1004). Picking 2-3 colonies per guides was sufficient to ensure a correct clone that was further confirmed by Sanger Sequencing.

Reverse-transcription PCR and quantitative PCR: RNA was extracted using the Qiagen Allprep Micro kit according to manufacturers instructions (Qiagen, #80204) and reverse transcribed using Verso cDNA Synthesis Kit Thermo Scientific™, cat. AB1453) according to the manufacturers recommendations. Quantitative reverse-transcription PCR was performed with Fast SYBR™ Green Master Mix (Applied Biosystems™, cat. 4385616) The reactions were performed in triplicate using 1/10 concentration of the cDNA obtained. Relative mRNA expression was normalized to GAPDH as an endogenous control using the 2-DDCT method.

T7E1 assay: To quantify genomic disruption frequency at the genomic level, samples were collected to extract genomic DNA by Qiagen Allprep Micro kit according to manufacturers instructions (Qiagen, #80204). Targeted regions were amplified by PCR from 100 ng of genomic DNA using Q5 Hot Start High-Fidelity 2× Master Mix (New England BioLabs, cat. M0494L) and KLF6 primers (forward: AGAGCTGGAACGTTACCTCCAG (SEQ ID NO: 13) and reverse: TCCCTCCAGGGCTGGTG (SEQ ID NO: 14)), −20 Kb Enhancer targeting (forward: TTGAGTTGGCAGTCTTTGCTTGG (SEQ ID NO: 15) and reverse: CCATGACTCGGTTTTGCTCT (SEQ ID NO: 16)), −25 Kb Enhancer targeting (forward: GGTGAACTGGGCTTACAGGA (SEQ ID NO: 17) and reverse: TAGAGCTAATTGTGGCT (SEQ ID NO: 18)) or −66 Kb Enhancer targeting (forward: GATTTGCTCAATTTGAAAG (SEQ ID NO: 19) and reverse: ACTGAAGATGAGATCACTGT (SEQ ID NO: 20) in a 25 uL reaction. PCR amplicon spanning the cleavage site was gradually hybridized in a thermal cycler. Hybridized fragments were then digested with 1.25 U of T7 endonuclease I (New England BioLabs, cat. M0302S) for up to 30 minutes at 37° C. Cleavage at heteroduplex mismatch sites were detected by agarose gel electrophoresis.

Myeloid and erythroid liquid culture: Cells were plated and cultured for 7 days under myeloid-promoting conditions: SCF (100 ng/ml), FLT-3 ligands (Peprotech, cat. 300-019), (10 ng/ml), IL-3 (Peprotech, cat. 200-003), (20 ng/ml), IL-6 (Peprotech, cat. 200-06), (20 ng/ml), GM-CSF (Peprotech, cat. 300-03), (20 ng/ml), and G-CSF (Peprotech, cat. 300-23), (20 ng/ml) and erythroid-promoting conditions: Epo (6 IU/ml) and SCF (100 ng/ml). At day 7 of myeloid and erythroid expansion, cells were stained for CD34 (BD Pharmigen, #304441), myeloid CD11b (BD Pharmigen, #301324), and erythroid CD71 (Pharmigen, #563769) and CD235a (BD Pharmigen, #559943) markers respectively, and also anti-KLF6 for cells transfected with sgKLF6 (Millipore, #MABN119) and goat-anti mouse IgG-AF594 (Thermo Fisher Scientific, #A-11032) or goat-anti-mouse IgG-AF488 (Thermo Fisher Scientific, #A-11059).

Small interfering RNA (siRNA) transfection: siRNA transfection was performed in HUDEP-2 cells, an immortalized human CD34+ hematopoietic stem and progenitor cell derived erythroid precursor cell line. The day before transfection, 5×10⁵ cells were plated and maintained in antibiotic-free culture medium. 25 nM of siRNAs (Dharmacon, ERG: L-003886-00-0005, RUNX1 L-003926-00-0005, FLI1 L-3892-00-0005, Non targeting: D-001810-10) were transfected with jetPRIME transfection reagent (Polyplus, cat. 89129-924) according to supplier instructions for 72 hs.

Lentiviral production and transient KLF6 induction of human CD34+: 293FT cells were maintained according to supplier instructions (ThermoFisher Scientific). dCas9-VP64 (addgene #53192) lentiviral particles were produced by co-transfection with packaging plasmids psPAX2 and pMD2.G using polyethylenimine transfection reagent (Polysciences). Lentivirus containing supernatant was collected 48-72 hours post-transfection, filtered through a 0.45-μm syringe filter, and concentrated using PEG-it virus precipitation solution (System Biosciences, #LV825A-1). Primary human CD34+ cells were freshly isolated from mobilized peripheral blood were purchased from Fred Hutchinson Hematopoietic Cell Procurement Services. Lentiviral transduction of CD34+ cells was performed in the presence of 8 μg/ml protamine sulfate (Sigma Aldrich, cat. P4020-1G). Four days post transduction, cells were sorted for CD34 and GFP double-positive cells on a BD FACS SORP Aria Fusion (BSL-2). After sorting, cells were cultured in stem-cell-promoting media (IMDM+20% Human Serum AB+hTPO (100 ng/ml), hSCF (100 ng/ml), hIL-6 (20 ng/ml), hFLT-3 (10 ng/ml) and non-essential amino acids) under low oxygen conditions for 48-72 hs where sgRNAs expression constructs were transfected in the presence of Fugene HD (Promega, cat. E2311). A non-targeting sgRNA was used as control. After 24-48hs in culture, cells were used for RNA extraction, CFU assay, or plated in myeloid: SCF (100 ng/ml), FLT-3 ligands (10 ng/ml), IL-3 (20 ng/ml), IL-6 (20 ng/ml), GM-CSF (20 ng/ml), and G-CSF (20 ng/ml) and/or erythroid-promoting conditions: Epo (6 μl/m1) and SCF (100 ng/ml). At day 7 of myeloid and erythroid expansion, cells were stained for CD34 (BD Pharmigen, #304441), myeloid CD11b (BD Pharmigen, #301324), and erythroid CD71 (Pharmigen, #563769) and CD235a (BD Pharmigen, #559943) markers respectively. Lentiviral titer was determined using LentiX GoStick (Takara, cat. 631280).

Stable KLF6 induction of human CD34+: 293FT cells were maintained according to supplier instructions (ThermoFisher Scientific). Lenti sgRNA(MS2)_zeo backbone (addgene #61427) containing the cloned sgRNAs and dCas9-VP64 (addgene #53192) lentiviral particles were produced by co-transfection with packaging plasmids psPAX2 and pMD2.G using jetPRIME transfection reagent (Polyplus, cat. 89129-924). Lentivirus containing supernatant was collected 72 hours post-transfection, filtered through a 0.45-μm syringe filter, and concentrated using LentiX concentrator virus precipitation solution (Takara Bio, cat. 631231). Lentiviral transduction of CD34+ cells was performed in the presence of 8 μg/ml protamine sulfate (Sigma Aldrich, cat. P4020-1G) as previously described with minor modifications [81]. Briefly, cells were washed twice in PBS 2% FBS at 200 g for 5 minutes. Cell supernatant was resuspended in 1 mL of the growth medium; and the cells were counted and then centrifuged again. Supernatant was removed and 50 to 100 μL of each viral solution resuspended in 1×PBS 2% FBS was added to the cell pellet (with multiplicity of infection=20 from each viral component to transduce). Virus and cells were carefully mixed avoiding the generation of bubbles to obtain a homogenous cell suspension and an equal amount of growth medium was added to the suspension. Cells were carefully mixed for 4 minutes at room temperature and then incubated at 37 C for 15 minutes. After 15 minutes of incubation cells were washed with 1×PBS 2% FBS and counted. Viral incubations were repeated 5 times. Four days post transduction, cells were sorted for CD34 and GFP double-positive cells on a BD FACS SORP Aria Fusion (BSL-2). After sorting, cells were cultured in stem-cell-promoting media (IMDM+20% Human Serum AB+hTPO (100 ng/ml), hSCF (100 ng/ml), hIL-6 (20 ng/ml), hFLT-3 (10 ng/ml) and non-essential amino acids) under low oxygen conditions for 12 hs. 24 hs after sorting cells were washed 1× with PBS 2% FBS and counted. Cells were plated at 1e106 cells per mL of media and 25 ug/mL Zeocin (InvivoGen. cat. ant-zn-1) was added immediately. Concentration for Zeocin selection was determined using a kill curve. Media was refreshed at 48 hs after starting Zeocin selection. The duration of the selection was 72 hours. Lentiviral titer was determined using LentiX GoStick (Takara, cat. 631280).

RNA-seq: RNA from transfected CD34+ cells (n=3 biological replicates per condition) was extracted using the Qiagen Allprep Micro kit according to manufacturers instructions (Qiagen, #80204). For the study of the E1 enhancer and KLF6-a, stranded libraries with ERCC spike-in controls were prepared by the University of Miami Oncogenomics Sequencing Core using the Illuminia TruSeq Stranded Total RNA kit (Illumina, #20020596) and Kapa HiFi Ribo depletion kit (#80981440702), respectively. Libraries were sequenced on the Next-Seq 500 with 75 bp paired-end sequencing.

RNA-seq alignment: Using Cutadapt (version 2.6), all reads were trimmed to 73 basepairs and adapters were removed [82]. Reads were aligned to the hg19 gencode v19 reference genome using the STAR aligner (version 2.7.3), specifying the following parameters: --outFilterType BySJout --outFilterMultimapNmax 20 --alignSJoverhangMin 8 --alignSJDBoverhangMin 1 --outFilterMismatchNmax 999 --alignIntronMin 20 --alignIntronMax 1000000 --alignMatesGapMax 1000000 --readFilesCommand gunzip -c --outSAMtype BAM SortedByCoordinate --outWigType bedGraph --outWigStrand Stranded --outWigNorm RPM --alignEndsType EndToEnd [83].

Differential gene expression analysis and GSEA: Gene counts were calculated using QoRTs (v1.3.0) [84]. QoRTs was run in first stranded mode using the hg19 gencode annotation file without entries for ribosomal RNA. Differential gene expression analysis was performed using DESeq2 v1.30.0 [85]. For activation controlled for enhancer KO and activation experiments, a multifactor design was used in order to control for experimental batch effects or cell source of donor, respectively. Dispersions were calculated using samples from both age groups and then contrasts were established for pair-wise comparisons. The log-fold change was corrected using the ashr method [86]. Significant genes were defined as having a fold change >2.0 and p-adjusted <0.05. Regularized log-counts (rld) and the wald-statistic were generated with DESeq2. For GSEA (v4.0.3) [87], the Wald statistic ranked list was used with the c2.all.v7.4.symbols gene set using the weighted enrichment score. For the heatmap of significant gene sets that change with HSCe aging, a previously generated Wald ranked list (CITATION for Adelman et al.) was ran with GSEA as above. The heatmap of normalized enrichment scores was plotted with ComplexHeatamp (CITATION), using distance correlation and the average clustering method. Gene sets that were not also significantly enriched in the KLF6-a comparisons were colored gray.

Colony-forming unit assay: Sorted CD34+ were seeded in methylcellulose, MethoCult H4435 (StemCell Technologies, #04435), in duplicate onto a 6-well SmartDish (StemCell Technologies, #27302) at 500 cells per condition. Colonies were scored on a STEMvision after 14 days of incubation (StemCell Technologies).

ChIP-seq: For ChIP-seq in cells with KLF6 KO or NTC, 50,000 FACS isolated human HSPC were used per immunoprecipitation. For ChIP-seq in young and aged cells with KLF6-a, 50,000 human HSPC were used per immunoprecipitation. ChIP-seq was then performed using the True MicroChiP (Diagenode, #C01010130) kit and that had been validated antibodies for specificity and reactivity using the MODified Histone Peptide Array (Active Motif, #13001). The manufacturers protocol was followed using the following modifications. After quenching with glycine and washing with PBS, samples were suspended in 100 μL undiluted Lysis buffer with 1× Diagenode protease inhibitor cocktail and 5 mM sodium butyrate per 50,000 cells. Samples were sonicated in 1.5 mL TPX tubes in a Bioruptor Pico for 6 cycles of 30 seconds on and 30 seconds off. Chromatin was immunoprecipitated for 12 hr at 4° C. using 0.5 μg H3K4me3 (Diagenode #C15200152, lot #A1052D), 0.5 μg H3K4me1 (Diagenode C15410194, lot #A1862D), or 0.5 μg H3K27ac (Abcam ab4729, lot #GR32117-41-2). After reverse crosslinking, DNA was purified using the minElute PCR Purification kit (Qiagen, #28004) and eluted in 16 μL of Tris-HCl ph 8.0. Enrichment was verified using QPCR with the primers GAGAGTCCTGGTCTTTGTCA (SEQ ID NO: 1) and ACAGTGCCTAGGAAGGGTAT (SEQ ID NO: 2) for H3K4me1, AGGGAGGGAATTAATCTGAG and ACAGTGCCTAGGAAGGGTAT (SEQ ID NO: 2) for H3K4me3, GAGCAGAGGTGGGAGTTAG (SEQ ID NO: 6) and TCAGACCCTTTCCTCACC (SEQ ID NO: 7) for H3K27ac, and TACTTGGTTTCTGCATCCTT (SEQ ID NO: 4) and TCACTAAAGAAACCGTTCGT (SEQ ID NO: 5) as a negative control for all marks. The remaining DNA was then used for library preparation with the AcceI-NGS 2S Plus DNA kit (Swift, #21024) with the Swift UDI, following the protocol for Input DNA <10 ng and modifying the Ampure cleanups after the Ligation I and Ligation 11 to do a 1:1 ratio. For the PCR amplification, a total of 16 amplification cycles was used. After the final Ampure bead cleanup DNA was eluted in 16 uL of Tris-HCl ph 8.0. Fold enrichment over input was then verified using QPCR with same primers as above. Multiplexed libraries were sequenced on a Novaseq with 100 bp single-end sequencing by the University of Miami Oncogenomics Sequencing Core.

ChIP-seq alignment: FastQC was used to evaluate library quality [88]. Illumina adapters were trimmed using Cutadapt (version 2.6) [89]. Reads were aligned to hg19 using Bowtie2 (version 2.3.5) [90]. Duplicate reads were flagged using Picard (version 2.21.2) with the command picard MarkDuplicates I=aligned_sort_name O=aligned_dup_name M=marked_dup_metrics ASSUME_SORTED=true REMOVE_DUPLICATES=false VALIDATION_STRINGENCY=LENIENT. Duplicate and multimapped reads were then removed with samtools (version 1.9) with the commands: samtools view -b -F 4 -q 5 and samtools view -b -F 1024 -L [91].

ChIP-seq analysis and differential peak calling: Peak calling for individual IPs was performed using the callpeak function from the Model Based Analysis of ChIP-seq 2 (MACS2 v.2.2.4) program [92]. Differential peak calling was performed using the R (version 4.0.2) package DiffBind (version 3.0.9) with the macs2 generated peaks [93]. For generating the count matrix, the bUseSummarizeOverlaps=TRUE feature was used and the DESeq2 method was used for differential analysis. Count matrices for young NTC and young KLF6-a, aged NTC and aged KLF6-a, and KLF6 KO and NTC were used. Significant differential peaks (FDR <0.05) were annotated to the nearest TSS of Refseq hg19 using the R package ChIP-enrich (v2.14.0) [94]. Genomic annotation of differential peaks was performed using the R-package Genomation (v1.22.0) [95], to hg19 genomic features or active and poised enhancers previously identified in young HSCe [21]. Heatmap s of differential peaks were plotted using the log 2(IP/Input) bigwig files generated using deepTools bamCompare and plotHeatmap functions [96]. Peaks were visualized using the UCSC genome browser. The macs2 SPMR and bdgcmp functions were used to generate fold enrichment ChIP-seq tracks that are normalized by read count and to the IP's corresponding Input.

In vivo transplantation of KLF6 KO, E1 enhancer targeted region and KLF6 induced HSPC: All experimental procedures performed on mice were approved by the Institutional Animal Care and Use Committees (IACUC) of the University of Miami. NSG-SGM3 (NOD.Cg-Prkdcscid112rgtmlWjlTg(CMV-IL3,CSF2,KITLG)1Eav/MloySzJ) mice were obtained from the Jackson Laboratory (Stock No. 013062) and bred inhouse for one generation. Before transplantation, young adult NSG-SGM3 female mice were preconditioned by sub-lethal total-body irradiation (1 Gy). Mice were then injected intravenously with 100,000-200,000 KLF6 deficient, −20 Kb enhancer targeted region, KLF6 induced or NTC human HSPCs manipulated as described above.

Flow cytometry analysis and cell sorting: Peripheral blood obtained through submandibular vein bleeding was collected in 0.5 M EDTA previously rinsed polypropylene tubes containing 5 uL of 0.5 M EDTA. Mice were observed for 30 minutes following blood collection for normal behavior and haemostasis. Bone marrow cells were obtained by flushing and dissociating using a 1-ml syringe with PBS via a 21-gauge needle. Spleens were placed in a cell strainer on a bacterial plate and processed by mechanical disaggregation until there was just connective tissue on the cell strainer. Spleen samples were maintained on ice until disaggregation of all the treated sample was completed. Single cell suspensions were prepared using standard methods from peripheral blood, bone marrow and spleens. Red blood cells were lysed using ACK buffer (Quality Biological, cat. 10128-802). The following antibodies were used for linage peripheral blood analysis and cell sorting: mouse CD45-APC (BioLegend, cat. 103112, clone 30-F11, dilution 1:200), human CD45-APC-Cy7 (BioLegend, cat. 304014, clone HI30, dilution 1:400), human CD3-PE-Cy5 (BioLegend, cat. 300410, clone UCHT1, dilution 1:200), human CD19-PE-Cy7 (BioLegend, cat. 363012, clone SJ25C1, dilution 1:200), human CD33-PE (BD Biosciences, cat. 555450, clone WM53, dilution 1:400). Hematopoietic and progenitor stem cell compartments from bone marrow single-cell suspensions were analyzed and sorted with the following antibodies: mouse CD45-APC (BioLegend, cat. 103112, clone 30-F11, dilution 1:200), hCD45-PE-CF594 (BioLegend, cat. 562279, clone H130, dilution 1:200), CD34-APC-F750 (BioLegend, cat. 343536, clone 581, dilution 1:100), CD38-PE-Cy7 (BioLegend, cat. 335790, clone HB7, dilution 1:100) CD10-BV421 (BioLegend, cat. 335790, clone HI10a, dilution 1:200), biotin mouse anti-human CD135 (BD Bioscience, cat. 624008. clone 4G8, dilution 1:200), brilliant violet 605 streptavidin (BioLegend, cat. 405229, dilution 1/300), CD90-PE (BioLegend, cat. 555596, clone 5E10, dilution 1:100), CD45RA-FITC (BioLegend, cat. 555488, clone HI100, dilution 1:100), CD49f-PE-Cy5 (BioLegend, cat. 551129, clone GoH3, dilution 1:100), CD71-APC (Thermo Fisher Scientific, cat. 17-0719-42, clone OKT9, dilution 1:100). Linage compartments from the spleen were analyzed and sorted with the following antibodies: mouse CD45-PB (BioLegend, cat. 103126, clone 30-F11, dilution 1:200), human CD45-APC-Cy7 (BioLegend, cat. 304014, clone H130, dilution 1:400), human CD3-PE-Cy5 (BioLegend, cat. 300410, clone UCHT1, dilution 1:200), human CD19-PE-Cy7 (BioLegend, cat. 363012, clone SJ25C1, dilution 1:200), human CD33-PE (BD Biosciences, cat. 555450, clone WM53, dilution 1:400). SYTOX™ Blue Dead Cell Stain (Thermo Fisher Scientific, cat. S34857) was used for exclusion of dead cells. Samples were washed with PBS 1% FBS before being analyzed by flow cytometry with a FACS SORP Aria Fusion (BD Biosciences) and analyzed using FlowJo software package (TreeStar).

Immunofluorescence assays: After washes with 1×PBS, cells were fixed with 2% paraformaldehyde in 1×PBS for 10 minutes at RT, washed twice with PBS and bound to poly-L-lysine coated slides. After 5 min fixation with 2% formaldehyde, cells were washed twice with 1×PBS for 5 minutes followed by incubation with Blocking Solution (1 mg/ml BSA, 3% goat serum, 0.1% Triton X-100 in PBS) for 30 minutes at RT. Next, cells were incubated with the primary antibodies in Blocking Solution for 1 hour at room temperatures, washed three times with 1×PBS, and incubated with Alexa 546 or 674 coupled antibodies (ThermoFisher Sc.) for another 30 minutes. Finally, cells were incubated with 0.5 [symbol] g/ml 4′, 6-diamino-2-phenylindole (DAPI) (Sigma) in 1×PBS for 5 minutes, washed again with 1×PBS for 10 minutes, air dried, and mounted with SlowFade Diamond antifade mounting reagent (ThermoFisher Sc.). Samples were analyzed using a Leica DM16000B microscope with LASX software (Leica). Antibodies to the following proteins were used in this study: 53BP1 (Millipore), and [symbol] -H2AX (Ser 139) clone JBW301 (Millipore).

Senescence-associated β-galactosidase staining: Senescence-associated b-galactosidase (SA-b-gal) staining was performed using the senescence-b galactosidase staining kit (Takara, cat. 631780) according to the manufacturers instructions. In brief, the cells washed twice with PBS, fixed and stained by the reagents provide by the kit.

Total and mitochondrial reactive oxygen species (ROS): Total (tROS) was assessed with the Total Reactive Oxygen Species (ROS) Assay Kit (Invitrogen, cat. 88-5930) following manufacturer instructions. Briefly, after two washes with 1×PBS, cells were incubated with Total Reactive Oxygen Species (ROS) Assay Kit for 60 minutes at 37 C and 5% CO2. After incubation, cells were washed with 1×PBS and analyzed by flow cytometry. Mitochondrial (mtROS) associated ROS levels were measured using the MitoSox™ Red mitochondrial superoxide indicator (Invitrogen, cat. M3600*) as describe before [97]. Briefly, cells were washed twice with 1×PBS, stained and incubated with MitoSox at 2.5 μM for 30 minutes at 37 C. Following incubation cells were washed with 1×PBS and analyzed by flow cytometry. Cells were analyzed in a CytoFLEX Flow Cytometer (Beckman-Coulter).

Western blot: Chromatin-bound proteins from HL-60, K562, KG-1, MV4-11, THP-1 and U937 cell lines were extracted using the Subcellular Protein Fractionation Kit for cultured cells (Thermo Scientific, #78840) following manufacturers instructions. Total protein concentration was quantified using BCA Protein Assay Kit (Thermo Scientific, #23227) according to manufacturer's instructions. Equal amounts of proteins per sample (40 rig) were denatured at 95° C. for 5 minutes. Samples were loaded into a 4-12% Bis-Tris gel (Thermo Scientific, #NP0322BOX), and then transferred to a nitrocellulose membrane. Samples were incubated with primary antibody at 4° C. overnight in TBS-Tween 5% milk and secondary antibody in TBS-Tween at room temperature for 1 hour. Antibodies used were as follows: mouse anti-KLF6 (EMD Millipore, #MABN119, clone 12A8.3, lot 3291308, 1/1,000 dilution), mouse anti-Histone H3 (Abcam, #ab10799, 1/2,000 dilution) and goat anti-mouse IgG, HRP-linked (Santa Cruz Biotechnology, #SC2031, lot J1414, 1/10,000 dilution). Chemiluminescent detection of horseradish peroxidase-conjugated secondary antibodies was performed using the Immobilon Western Chemiluminescent HRP Substrate (EMD Millipore, #WBKLS0100). Densitometry was performed using the LI-COR Image Studio software. For all samples, intensity of KLF6 was normalized to that of a loading control, histone H3. All cell lines were authenticated using STR profiling.

STATISTICAL ANALYSIS: For genome-wide sequencing assays, we corrected for multiple testing, and used q<0.05 as a cutoff to determine significance, for all other statistical tests, we used a p-value cutoff of p<0.05 to define significance. In the figures, * means p<0.05, **p<0.01, ***p<0.001, and ****p<0.0001. Statistical analysis were performed using Prism software (v7.0). For time-series data, we fitted a linear mixed-effects model with the parameter examined (e.g. number of CD33+ cells) was fitted as a function of time and condition (e.g. KLF6 KO and NTC). Then, we used anova on the fitted model to examine whether the examined parameter significantly differed between the two conditions examined. Calculations were done in R with the nlme package.

Results

Loss of KLF6 In Vivo Recapitulates Features of Physiological Hematopoietic Aging

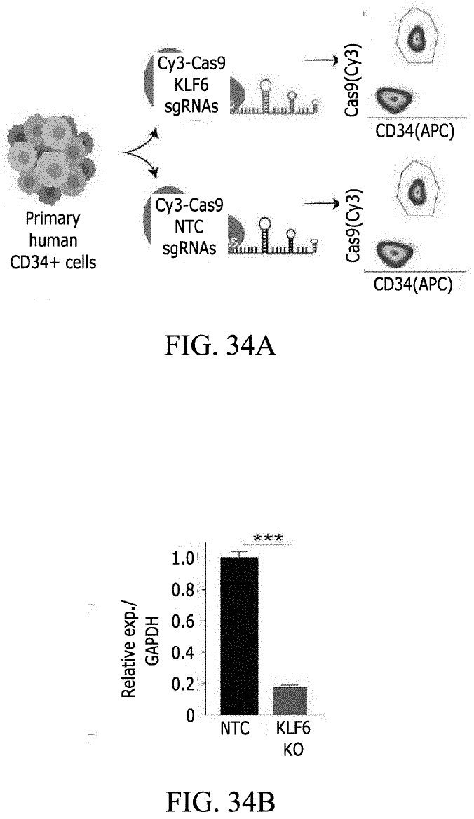

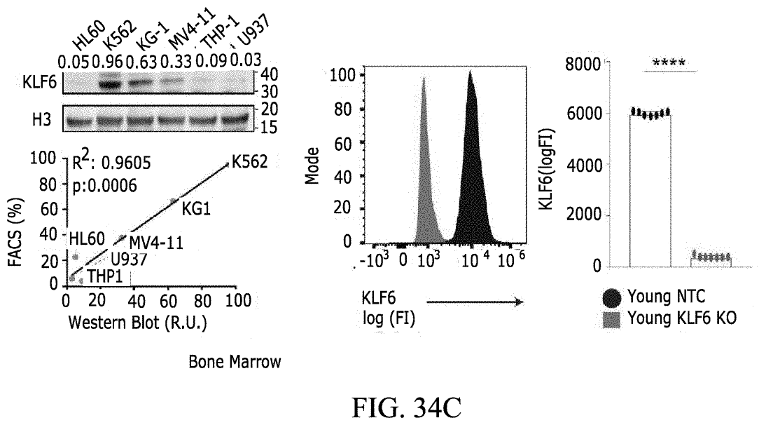

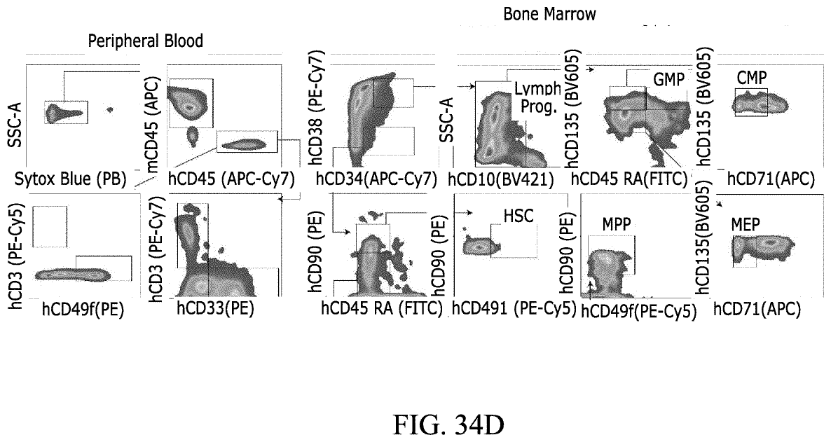

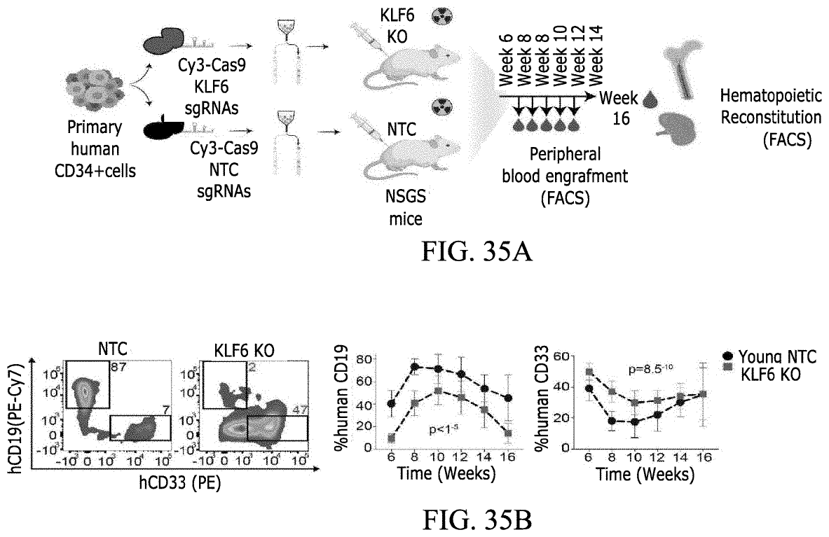

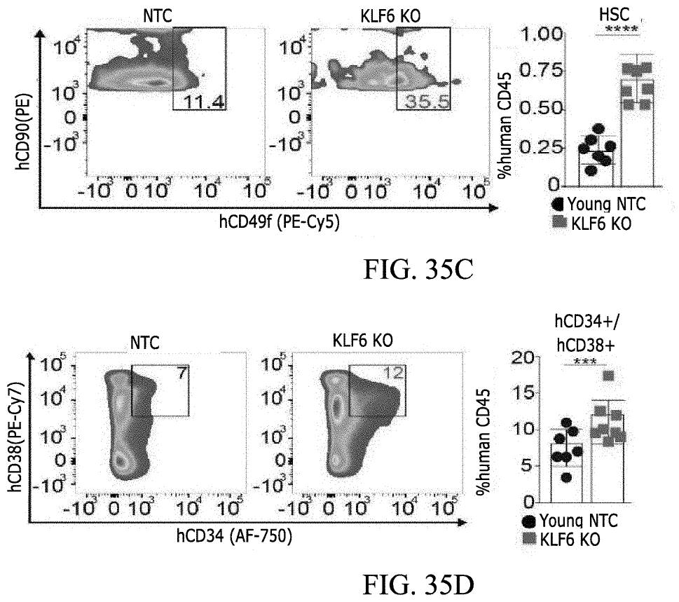

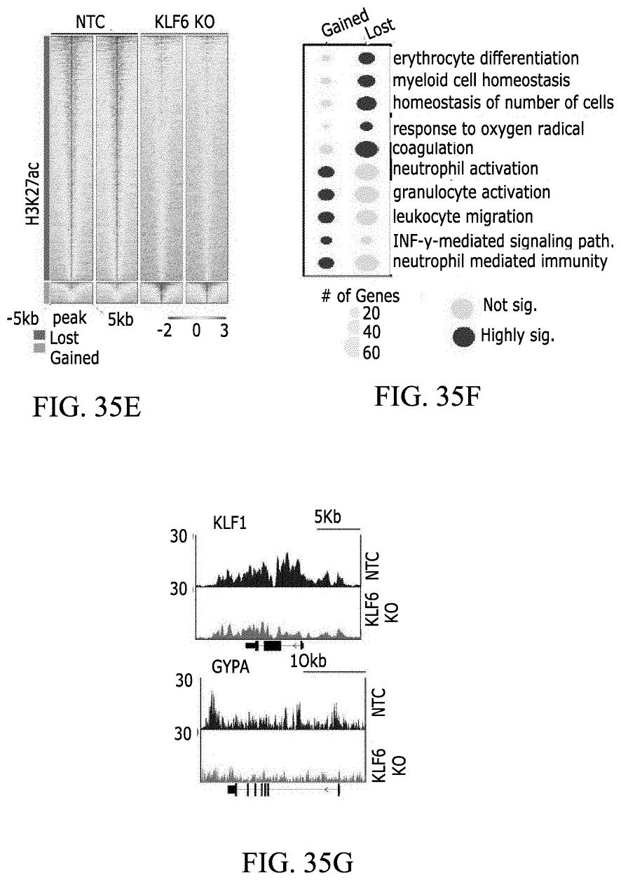

Based on our previous observations, we sought to further understand the role of KLF6 in hematopoiesis and determine whether its loss in HSPCs impairs normal hematopoiesis in vivo. For this purpose, we utilized a CRISPR-Cas9-mediated editing approach of the KLF6 locus in primary human hematopoietic stem and progenitor cells (CD34+) cells (FIG. 34) [37, 38]. Transfection of HSPC from young donors (26-28 years of age) with KLF6-sgRNA (henceforth, KLF6 KO) resulted in efficient downregulation of KLF6 expression both at the mRNA and protein levels compared to cells transfected with non-targeting control (NTC)-sgRNA (henceforth, NTC) (Figure S1B and Figure S1C). 1.6×10⁵ KLF6 KO or NTC cells were then transplanted into immunodeficient NSG-SGM3 (NOD.Cg-Prkdcscid112rgtm1WjlTg(CMV-1L3,CSF2,KITLG)1Eav/MloySzJ) mice (FIG. 35A) and transplanted animals monitored closely for engraftment and donor contribution to hematopoiesis.

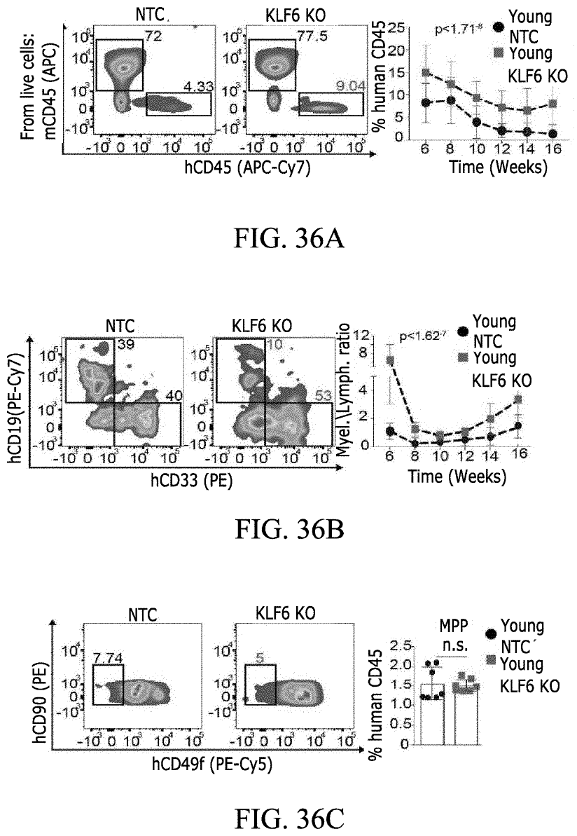

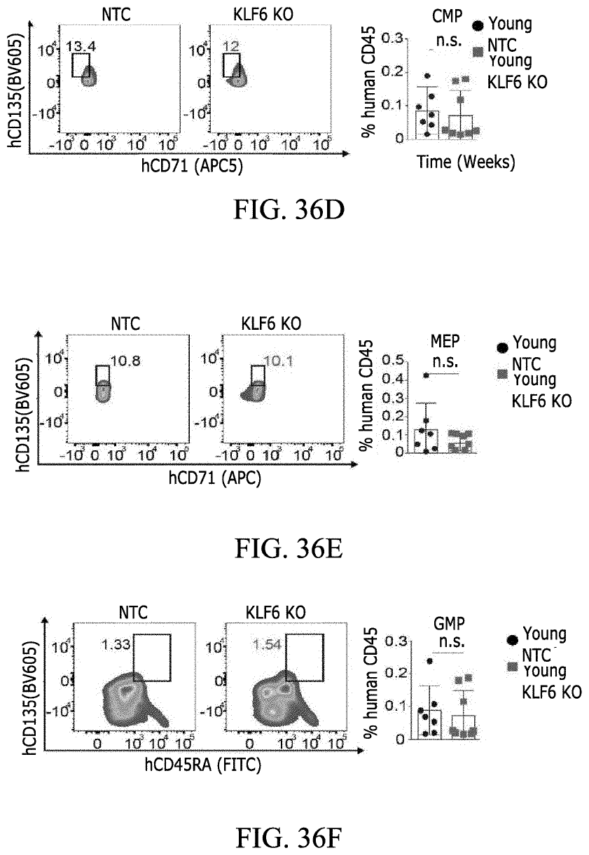

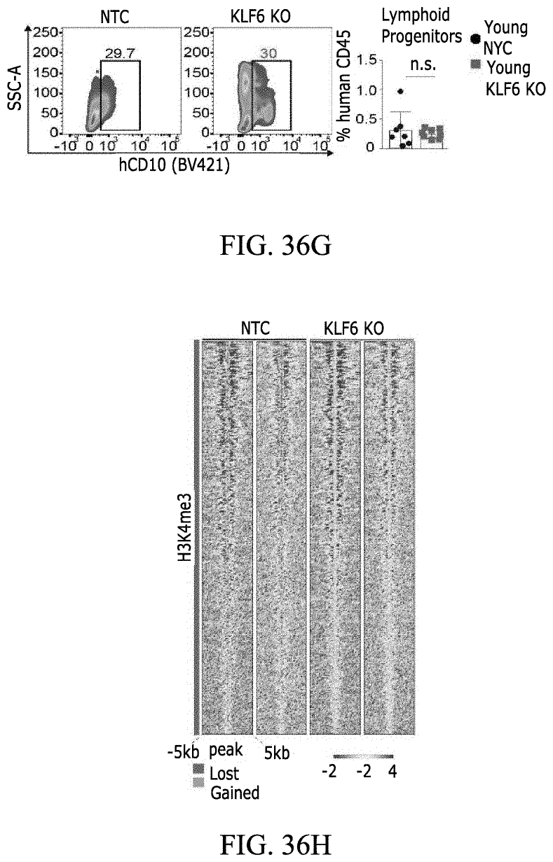

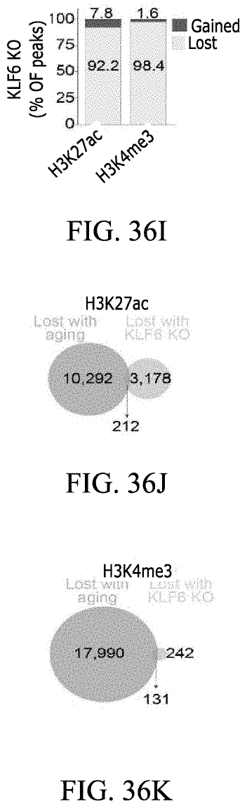

Examination of peripheral blood engraftment (measured as percent of human CD45+ cells) showed that >1% human cells were present in both the KO and NTC groups at week 6 and continuously throughout the entire experiment (FIG. 34D and FIG. 36A). Notably, lineage reconstitution analysis of peripheral blood showed significantly less contribution to the lymphoid fraction (hCD45+, CD19+, CD33−; p<1e-5, Linear mixed-effects model) and a concomitant increase in contribution to myeloid output (hCD45+, CD19−, CD33+; p<1.14-10) in mice injected with KLF6 KO cells relative to the NTC group between weeks 6 and 16 post-injection (FIG. 35B), resulting in an increased myeloid to lymphoid ratio (CD33+ to CD19+) (p<1.62e-7), a feature reminiscent of HSC aging (FIG. 36B).

To determine if KLF6-dependent effects on myeloid and lymphoid reconstitution were due to phenotypic alterations within the stem cell compartment, we performed a detailed analysis of the stem and progenitor compartments within the bone marrow 16 weeks post-transplantation. Analysis of immunophenotypically defined HSC (CD34+, CD38−, CD45RA−, CD90+, CD49f+) [39] within the human (hCD45+) fraction of the bone marrow revealed an increase in hHSC frequency (p<0.0001, two tailed t-test) and expansion in the CD34+, CD38+ progenitor compartment (p=0.0285) in the KLF6 KO group compared to NTC (FIG. 35C-D and FIG. 34D). Analysis of specific progenitor fractions did not reveal any significant differences in either multi-potent progenitors (MPPs) (CD34+CD38−CD90−CD45RA−CD49f−) (FIG. 36C), common myeloid progenitors (CMPs) (CD34+CD38+CD10−FLT3+CD45RA−) (FIG. 36D), megakaryocyte-erythroid progenitors (MEPs) (CD34+CD38+CD10−FLT3−CD45RA−) (FIG. 36E), granulocyte monocyte progenitors (GMPs) (FIG. 36F) (CD34+CD38+CD10−FLT3+CD45RA+) or lymphoid progenitors (CD34+CD38+CD10+) (FIG. 36G). Thus, loss of KLF6 in young human HSPCs recapitulates the aging-related myeloid bias, resulting from decreased lymphoid contribution to peripheral blood, as well increased HSC and progenitor frequency.

We next sought to determine if epigenetic changes within KLF6 KO progenitors contribute to the observed aging-like phenotype. Using chromatin immunoprecipitation followed by sequencing (ChIP-seq) we evaluated the genomic distribution of H3K27ac and H3K4me3, two activating histone modifications we have previously observed are profoundly altered in aged human HSCe [21], in human KLF6 KO human HSPCs. (n=2 biological replicates). While minor changes were observed in H3K4me3 enrichment upon loss of KLF6 (n=373 lost and n=6 peaks gained, FDR <0.05), 3,390 peaks had reduced H3K27ac upon KLF6 depletion (FDR <0.05). This focal reduction in H3K27ac was accompanied by relatively modest gains at 285 peaks (FIG. 34E and FIG. 36H). Given that the epigenetic changes seem to be driven by H3K27ac, we focused our analyses on this histone modification.

Functional annotation showed that regions that have reduced H3K27ac with KLF6 KO are associated with myeloid cell homeostasis, erythroid differentiation, and response to oxidative stress, and include hematopoietic transcription factors such as GATA1/2, KLF1, GYPA, FOXO3, BCL11A, and the BCL2 family members BAK and BAX (FDR <0.05, Benjamini-Hochberg) (FIG. 34F-G).

While the number of regions that gain H3K27ac were modest, these regions were annotated to genes such as CD44, ABR, and HOXC4 and functional annotation showed they are associated with granulocyte and neutrophil mediated immunity (FDR <0.05, Benjamini-Hochberg) suggesting that though less widespread, these changes may still play a biologically relevant role (FIG. 341). Transcription factor binding motif analysis suggests that these regions may be regulated by the myeloid associated transcription factors PU,1 GABPA and ETS1 (FIG. 34J).

Given our findings, we next explored the degree of similarity between regions that display loss of these chromatin marks with normal human aging in the HSCe compartment and those that change with KLF6 disruption. While the direct overlap of significant differential peaks that lose H3K27ac or H3K4me3 with aging and with KLF6 loss was limited (n=212 peaks and n=131 peaks, respectively), select transcription factors, including NFIA, NFATC1, and SOX12 and the corepressor BCOR, as well as the epigenetic modifiers KAT6B and SIRT5, showed reduced H3K27ac with both aging and KLF6 perturbation (FIG. 36I-K). Together these results suggest that KLF6 is a key transcription factor involved in modulating normal HSPC developmental and differentiation programs and that loss of KLF6 results in the production of aberrant HSPC function, characterized by the presence of a myeloid-biased population and an expansion of stem and progenitor populations.

KLF6 is Regulated Via an Upstream Enhancer Located 20 kb from its Promoter

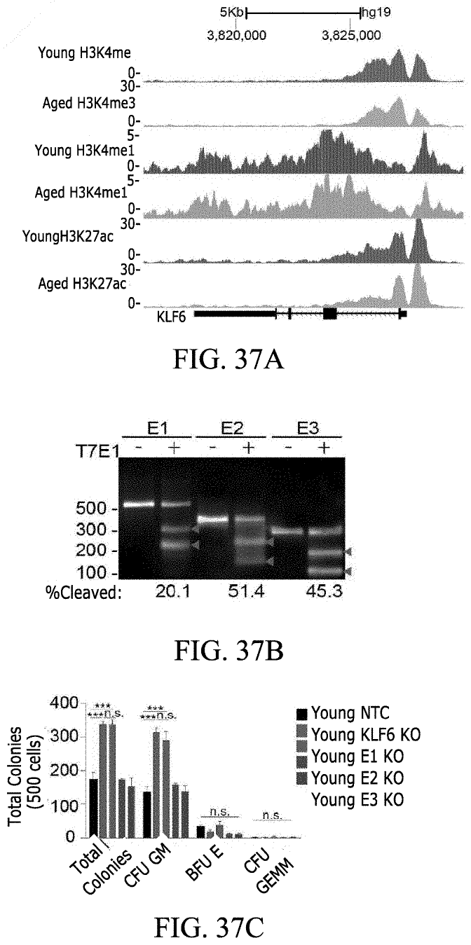

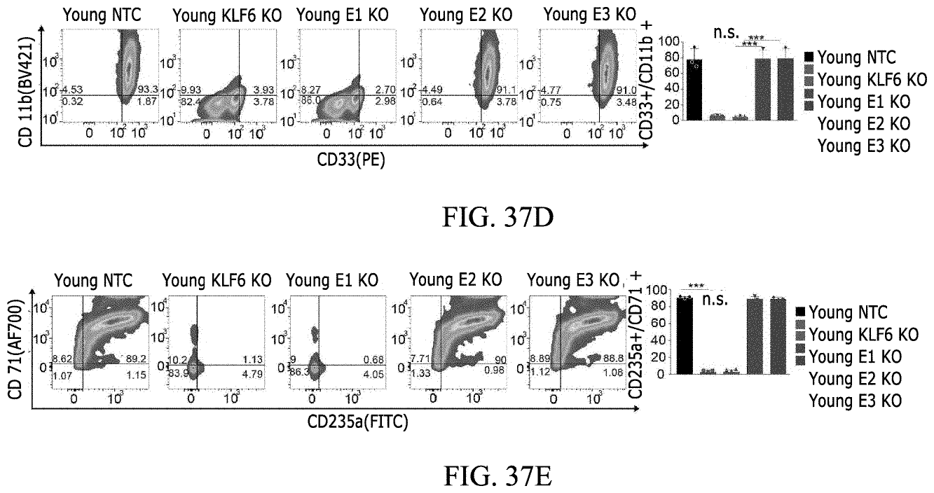

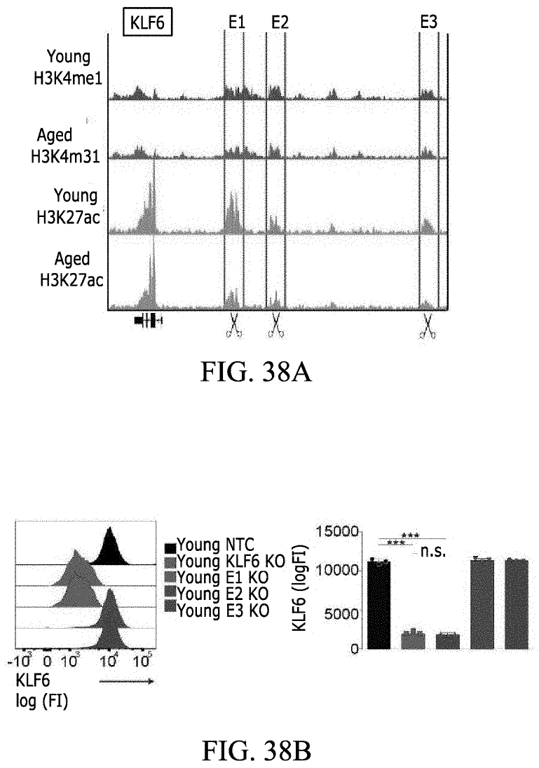

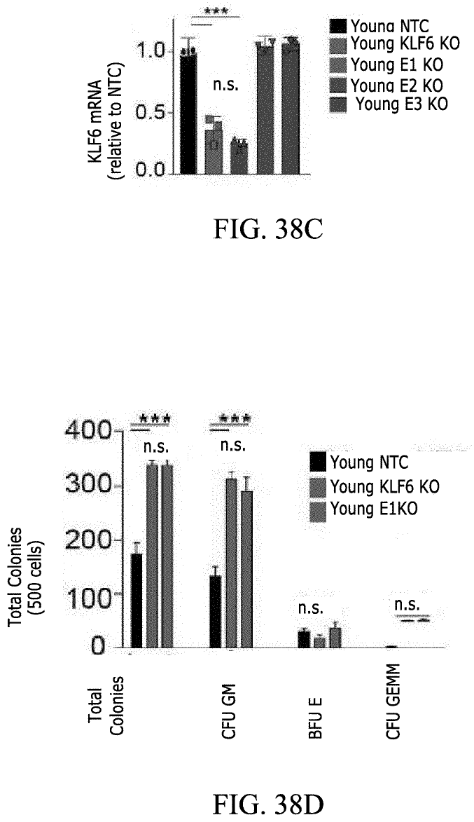

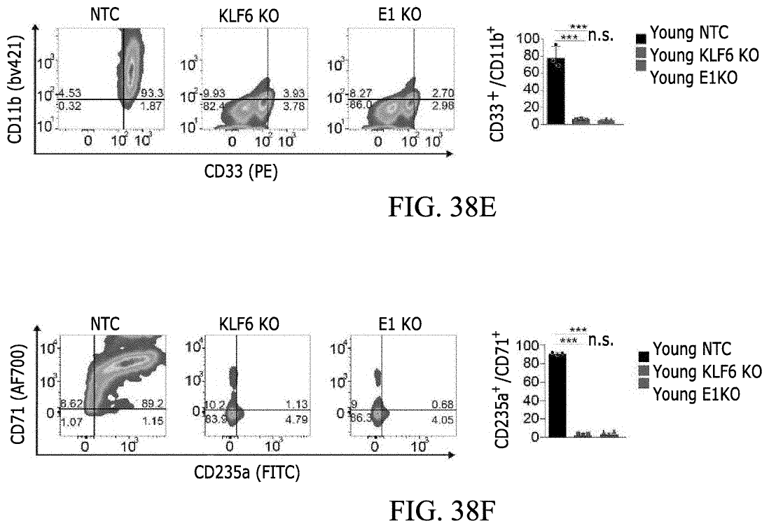

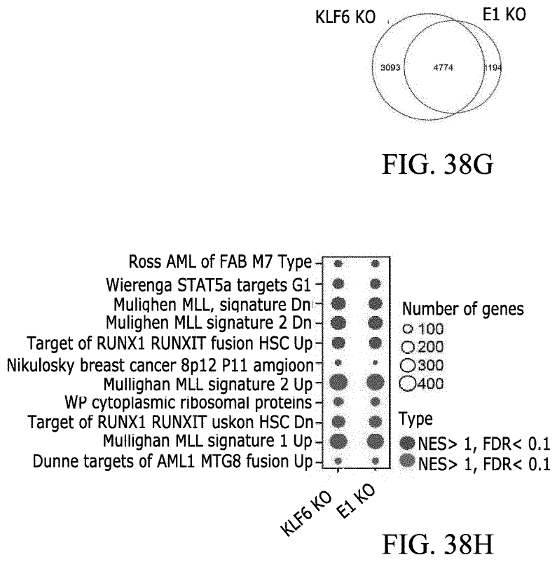

Given that our previous epigenomic analyses of human HSCe did not show any age-related changes in chromatin marks at the KLF6 promoter region (FIG. 37A), we reasoned that distal regulatory elements may be responsible for this downregulation. Analysis of the chromatin status surrounding the KLF6 locus, identified three putative enhancer regions marked by the presence of H3K4mel and H3K27ac at −20, −25 and −66 kb (henceforth, E1, E2, and E3; respectively) from the KLF6 transcription start site (TSS) Notably, all three of these regions displayed an age-related decrease in H3K27ac (FIG. 38A). We carried out CRISPR-Cas9-mediated editing of the three putative enhancer regions in normal CD34+ cells from young donors (<35 yrs). Genomic analysis with the T7 endonuclease I (T7EI) assay after CRISPR-targeting showed efficient editing of these three regions (Figure S3B). However, KLF6 expression analysis demonstrated that only targeting the E1 region located at −20 kb from the TSS (hereafter, E1 KO), but not those at either −25 kb or −66 kb (hereafter, E2 KO and E3 KO, respectively), resulted in reduced expression of KLF6 mRNA and protein levels (FIG. 38B and FIG. 38C). Targeting of the E1 enhancer region, but not the E2 or E3 regions, resulted in an increase in total colony numbers and granulocyte-monocyte colonies when plated on methylcellulose (p=0.0051), similar to that observed in KLF6 KO cells (FIG. 38 and FIG. 37C). We further tested the myeloid and erythroid differentiation potential of the targeted cells in liquid culture. Genomic editing of the E1 enhancer was sufficient to recapitulate the impairment of both myeloid and erythroid in vitro differentiation potential as seen in KLF6 KO cells (p<0.0001). In contrast, disruption of neither the E2 nor the E3 putative KLF6 enhancers resulted in any phenotypic consequences in the in vitro differentiation outputs of the edited cells, indicating that while these may play important regulatory roles in other contexts, they do not do so in HSPCs (FIG. 38E-F and FIG. 37D-E).

In order to determine if the observed phenotypic changes are due to the E1-20 kb enhancer regulating the same gene networks that are deregulated upon loss of KLF6, we performed RNA-seq in primary CD34+ cells (n=3 replicates), targeting the E1-20 kb enhancer. Differential gene expression analysis revealed 5,968 differentially expressed genes (DEG) in the E1-targeted cells compared to NTC (2,964 upregulated and 3,004 downregulated, DESeq2, FDR <0.05 and absolute fold-change >2). Comparison of these transcriptional changes to those seen after KLF6 KO, showed that the majority (n=4,774 genes) of the KLF6 gene regulatory network is also perturbed upon disruption of the E1 enhancer region (p<0.0001) (FIG. 38G). As expected, given the high overlap between the two signatures, gene set enrichment analysis (GSEA) using the E1 KO expression profile showed an enrichment for gene sets similar to those seen with the KLF6 locus disruption [21], including gene sets associated with leukemias and other malignant profiles (FDR <0.05) (FIG. 38G).

Using DNA motif analysis and publicly available transcription factor ChIP-seq data we had previously identified three candidate transcription factors, FLI1, ERG, and RUNX1, whose binding overlapped with the E1 enhancer and may regulate its function [21, 40]. Analysis of our previously published data set revealed that of these transcription factors, only FLI1 had a significant difference with aging (adjusted p-value <0.01 and absolute fold change >1.5). Notably, this transcriptional repressor was upregulated with normal aging (FIG. 38I). We next performed siRNA knockdown of each of these transcription factors in HUDEP-2 cells, an immortalized human hematopoietic stem and progenitor cell derived erythroid precursor cell line [41], and evaluated KLF6 mRNA levels by quantitative RT-PCR. Knockdown of ERG or RUNX1 did not result in any changes in KLF6 expression. However, FLI1 downregulation resulted in a marked upregulation of KLF6, suggesting that FLI1 binds to the E1 enhancer to regulate KLF6 expression levels (FIG. 38J). Together, this indicates that disruption of the E1 enhancer region results in comparable in vitro differentiation and colony-forming impairment, and transcriptional reprogramming to that seen with loss of KLF6, and that FLI1 changes during normal aging may be responsible, through regulation of this enhancer, for the downstream impact on KLF6 levels.

KLF6 Enhancer Disruption Promotes Aging-Like Features In Vivo

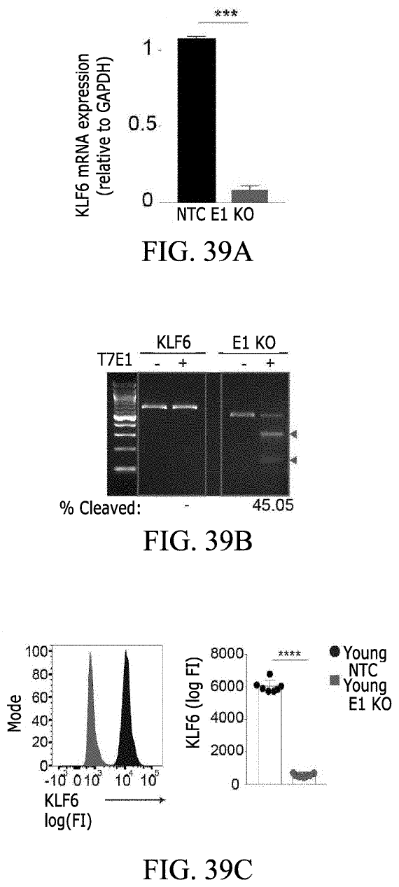

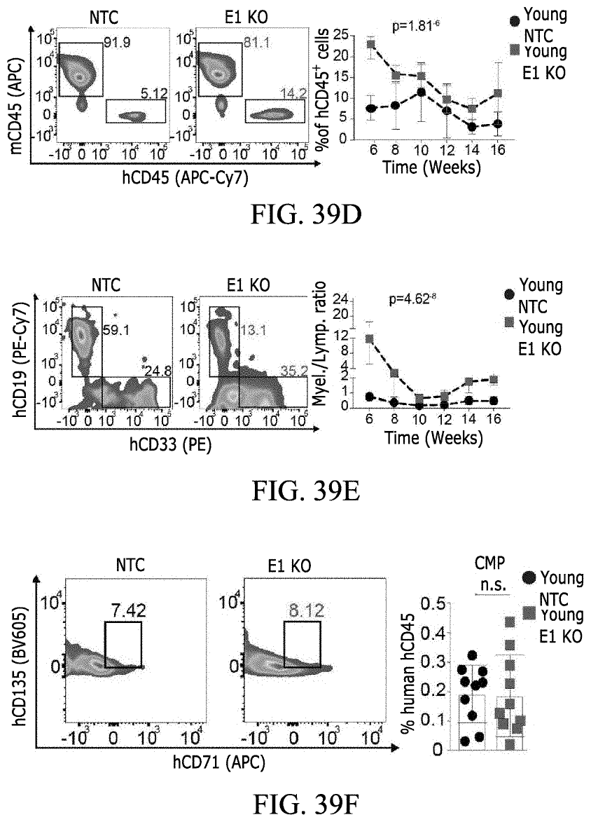

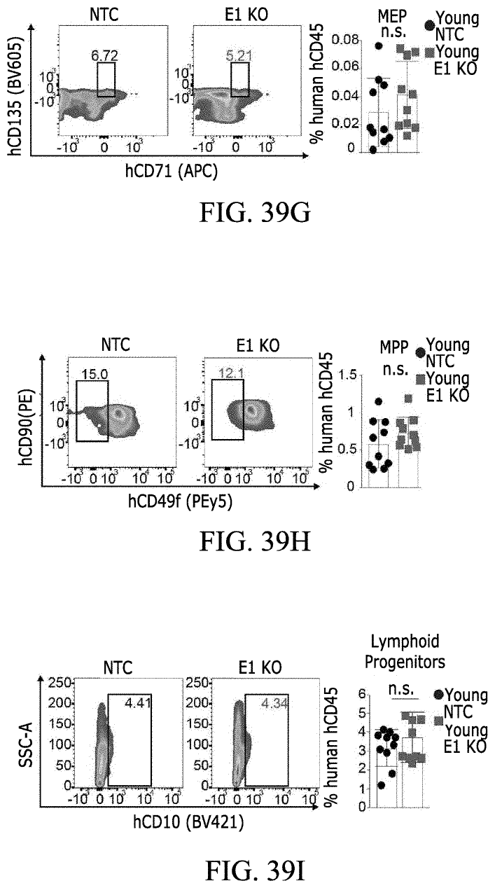

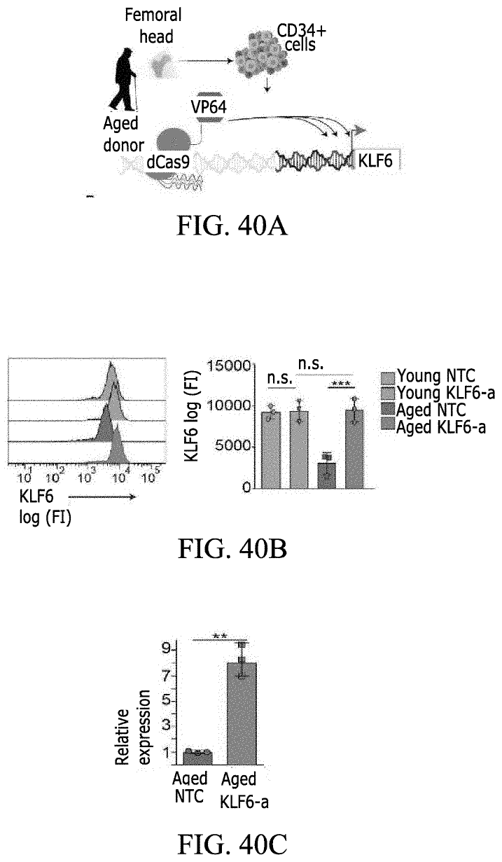

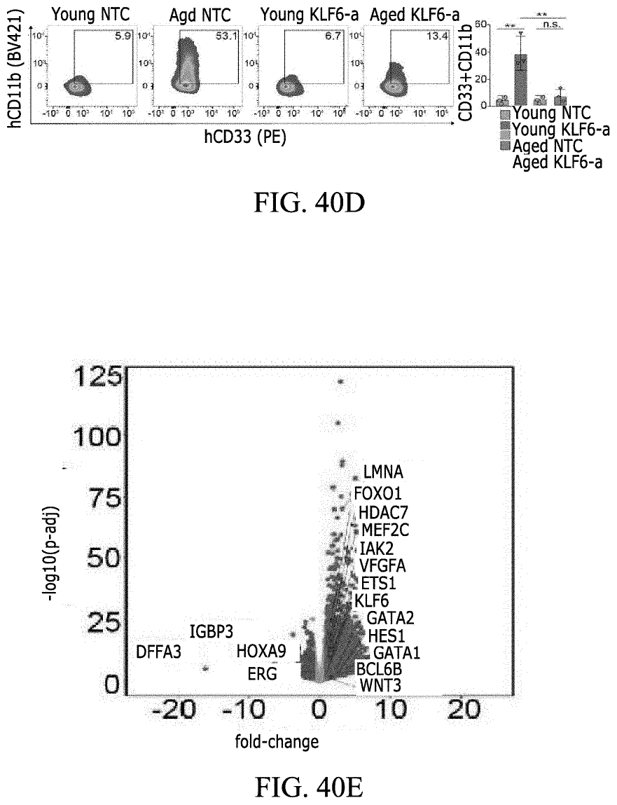

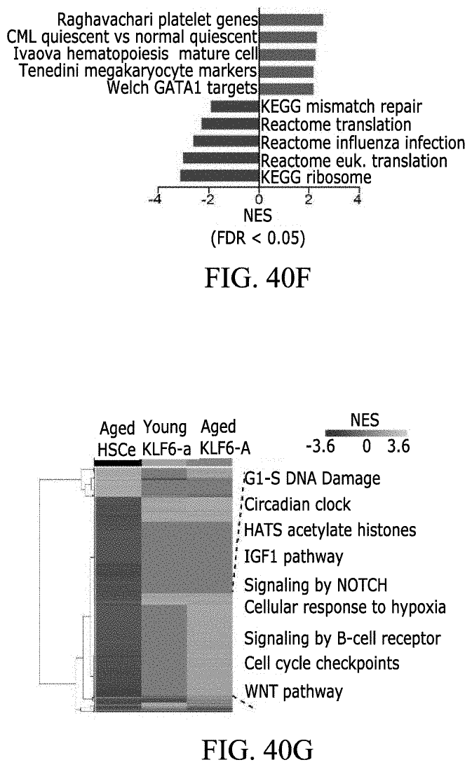

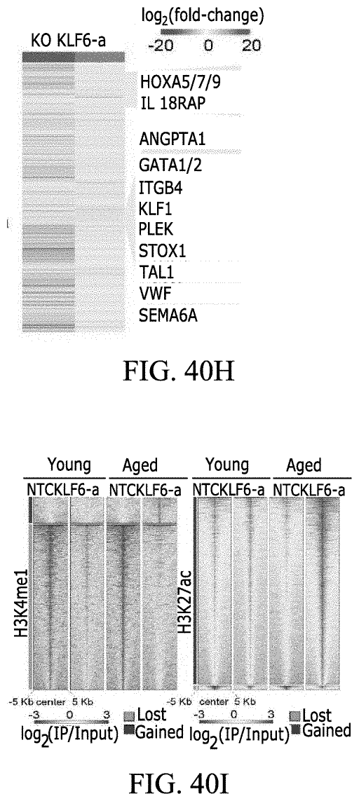

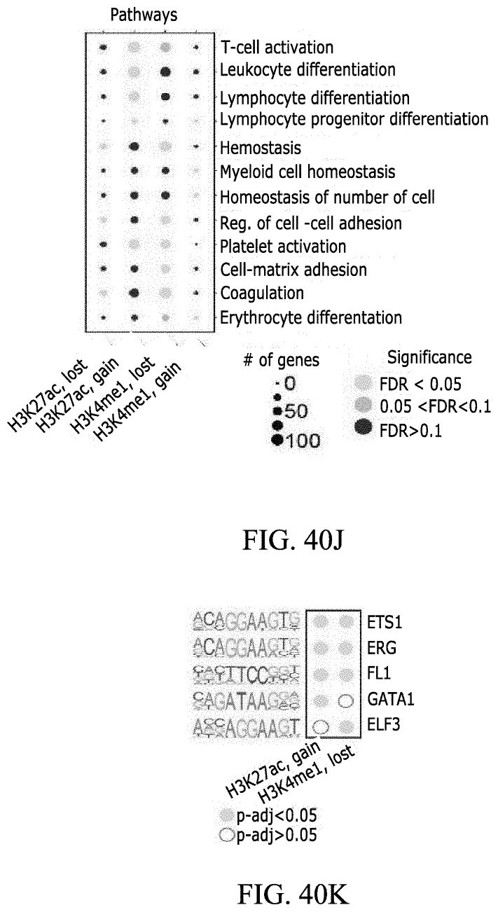

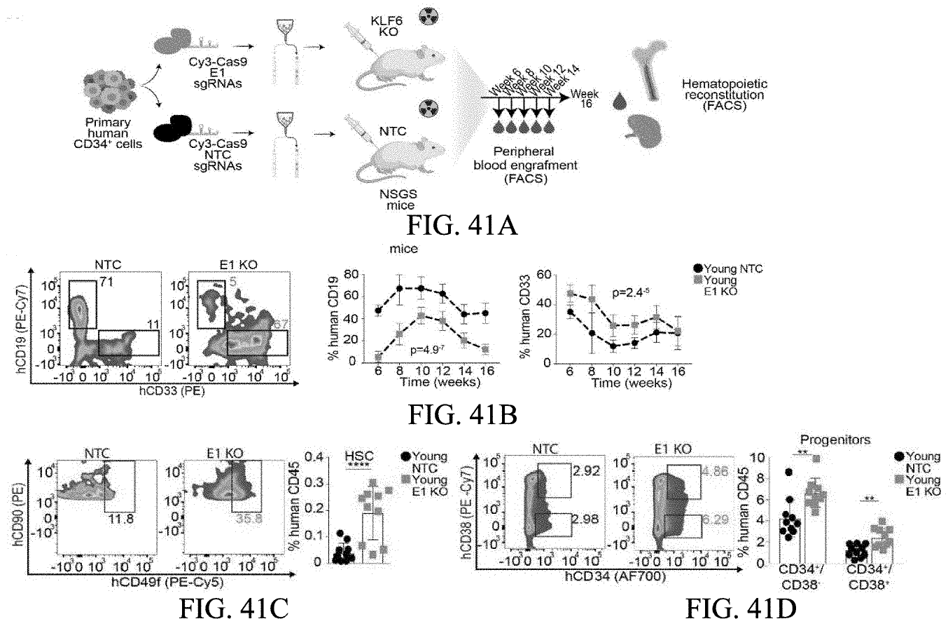

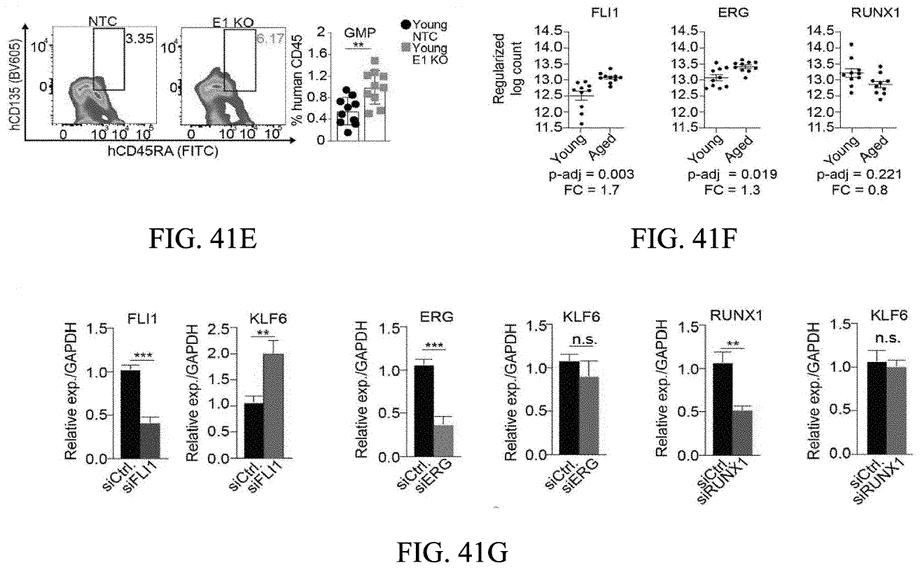

After confirming that disruption of the E1 enhancer phenocopies KLF6 disruption in vitro, we next wanted to determine if targeting this region could recapitulate the phenotypic consequences observed upon KLF6 disruption in vivo. For this purpose, we performed CRISPR-Cas9 genome editing at the E1-20 Kb region in primary human CD34+ cells isolated from young healthy donors (26-28 yrs.) and KLF6 depletion was confirmed (FIG. 39A-C). CD34+ cells with either disruption at the KLF6 E1 enhancer or NTC were injected into immunodeficient NSG-SGM3 mice (FIG. 40A). As before, we examined peripheral blood engraftment continuously throughout the experiment, observing >1% human cells were present in both the test and the control groups (FIG. 39D). Peripheral blood lineage reconstitution analysis of both groups revealed a significant reduction in the human lymphoid fraction (p<1e-5, Linear mixed-effects model) with an associated increment in the human myeloid output (p<1.14e-10) in the mice injected with E1 KO cells relative to the NTC group between weeks 6 and 16 post-injection with a concomitant increase in the myeloid to lymphoid ratio (p<1.62e-7) (FIG. 41B and FIG. 39E). Bone marrow was isolated for analysis at 16 weeks after transplant, at the end of the experiment. Deletion of the −20 Kb enhancer resulted in an expansion of the HSC compartment as compared to the NTC (p=0.0003) as well as an overall increase in the CD34+/CD38+ and CD34+/CD38− progenitor compartments (p=0.0022 and p=0.003, respectively) (FIG. 41C-D), and a specific expansion of the GMP (CD34+CD38+CD10−FLT3+CD45RA+) fraction (p=0.0029) (FIG. 41E. In addition, a trend to an increase in the CMPs, MEPs, MPPs and lymphoid progenitor compartments was observed, though these changes did not reach statistical significance (FIG. 40F-I).

Induction of KLF6 Expression Ameliorates Phenotypic Changes Associated with HSC Aging

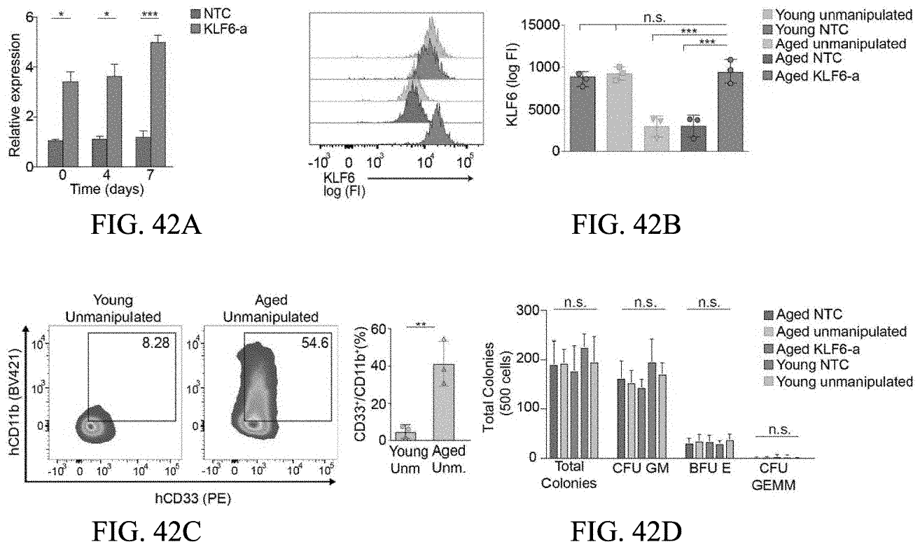

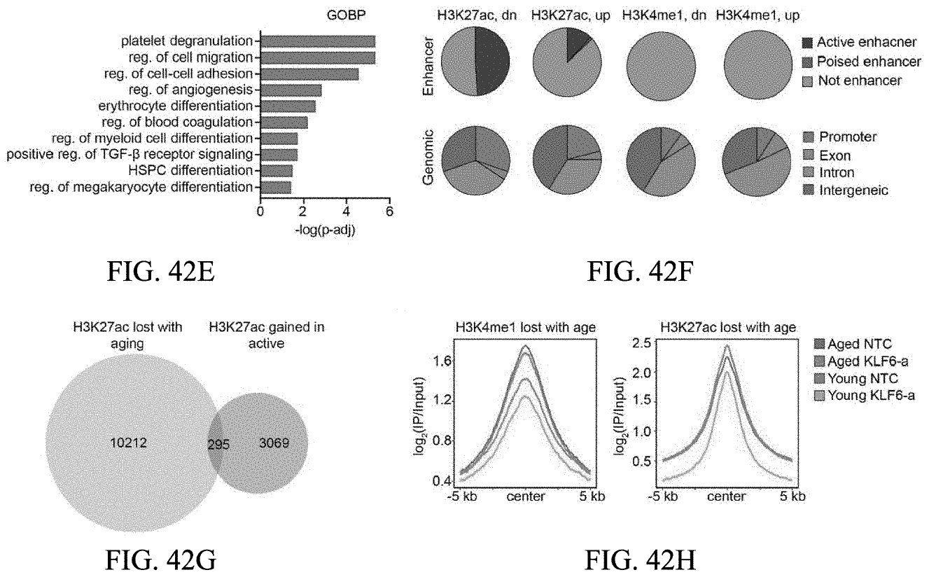

We next sought to test the hypothesis that re-establishment of KLF6 levels in aged HSPCs could revert features of HSC aging. For this purpose, we used a system containing a catalytically deactivated Cas9 (dCas9) fused to the VP64 transcriptional activator. CD34+ cells were isolated from the bone marrow of healthy individuals aged either 65-75 yr. or 18-35 yr. After lentiviral transduction with the pLV hUbC-dCas9 VP64-T2A-GFP (herein, dCas9-VP64) [42], the KLF6 promoter was targeted for activation through transfection of validated sgRNAs (FIG. 40A). Aged KLF6-activated (KLF6-a) CD34+ cells were compared to both non-targeting control sgRNA (NTC) or unmanipulated CD34+ cells from the same donor, as well as to young CD34+ cells targeted with KLF6 activation sgRNAs, NTC or left unmanipulated. Activation with dCas9-VP64 resulted in persistent KLF6 re-expression, even after 7 days in liquid culture, demonstrating that we have established a robust method for in vitro manipulation of KLF6 levels (p=0.0005) (FIG. 40B-C and FIG. 42A-B).

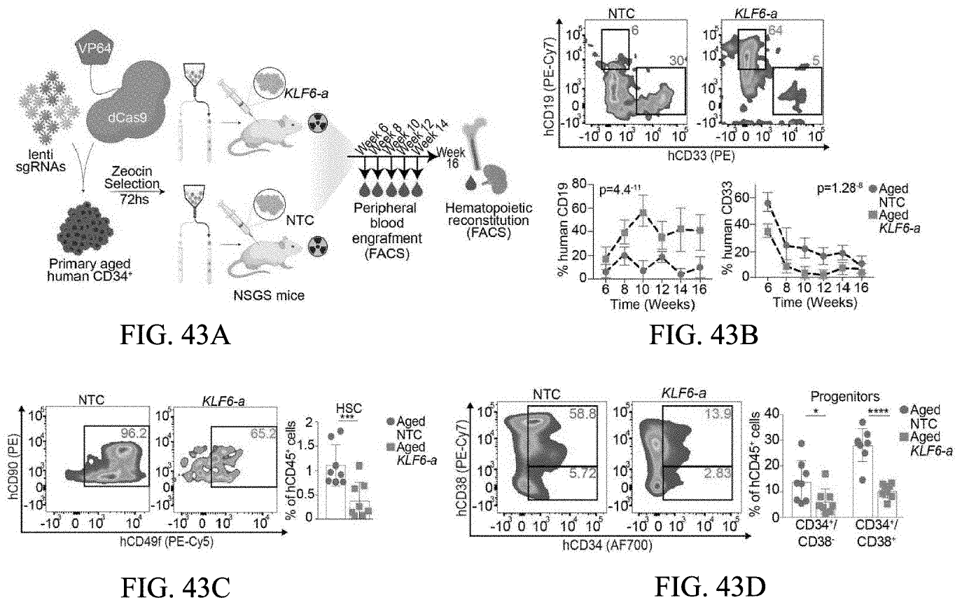

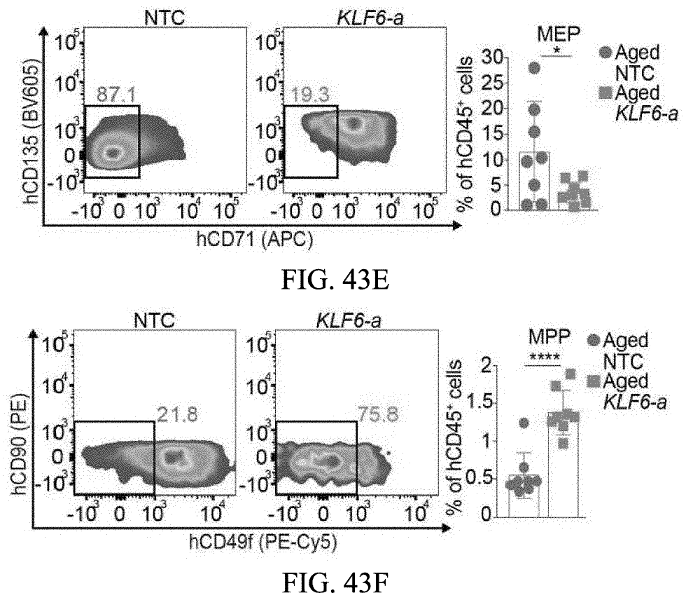

In order to determine whether KLF6 induction in aged HSPC had a revitalizing effect in these cells, we analyzed their myeloid differentiation potential. We first established a baseline differentiation behavior for normal aged CD34+ cells under myeloid differentiation conditions by comparing unmanipulated CD34+ cells from both young and aged healthy donors. Aged unmanipulated cells differentiated in vitro more efficiently into myeloid cells compared to their young counterparts, resulting in a greater proportion of CD33+ and CD11b+ cells (p<0.0041), while NTC had no impact in either age group. By contrast, when aged cells were induced to express KLF6, and compared to their NTC, they were found to display a myeloid differentiation potential that was closer to that of young control cells than to that of control aged cells (p<0.0035). Young KLF6-a cells, on the other hand, did not display any changes in differentiation compared to NTC, suggesting that KLF6-a has age specific effects (FIG. 40D and 43C).

Similarly to what was reported by Pang and colleagues, [8], when we compared the colony forming potential of CD34+ cells between young and aged healthy donors, no significant differences were detected between the two groups. In this context, KLF6 restoration in aged CD34+ cells had no impact on colony forming potential compared to either aged controls or their young counterparts (FIG. 42D).

We next performed RNA-Seq in aged NTC or KLF6-a HSPC, as well as young KLF6 induced cells in order to determine if the effects of KLF6 induction on gene expression are age-dependent (n=3 biological replicates per condition). Differential analysis identified 1,938 differentially expressed genes (DEG) in aged KLF6-a compared to aged control (FDR <0.05, absolute fold-change >2), with only 20% of these also being differentially expressed in their younger counterparts (n=761 DEG in young). Genes uniquely downregulated upon KLF6-a in aged but not young HSPC, included those associated with senescence and the senescence associated secretory phenotype (SASP) such as IGFBP3, ICAM3, PLA2R1, and TNFSF15, as well as the transcription factors ERG and GFI1, while GATA1/2, BCL6B, HES1/2, ETS1, and GFI1B were upregulated in KLF6-a HSPC (FIG. 40E). Gene set enrichment analysis revealed downregulation of genes involved in functions that are commonly altered with aging, such as translation and protein synthesis, DNA damage, and immune response [3, 9, 12, 43-45]. In contrast, KLF6-a resulted in upregulation of genes associated with the megakaryocytic lineage and hematopoiesis (GSEA, FDR <0.05) (FIG. 40F). Furthermore, comparison of gene sets to those that change with normal human HSCe aging, revealed that 652 of the gene sets that are significantly deregulated (FDR <0.1) with normal HSCe aging are enriched upon KLF6 activation in aged but not young HSPC. Notably, the majority of these genes sets, which are downregulated during normal aging, become upregulated with KLF6-a, including genes associated with DNA damage, cell-cycle, histone acetylation, and WNT and NOTCH signaling, indicating that KLF6 activation can revert the transcriptional program of aged HSPCs (FIG. 40G).

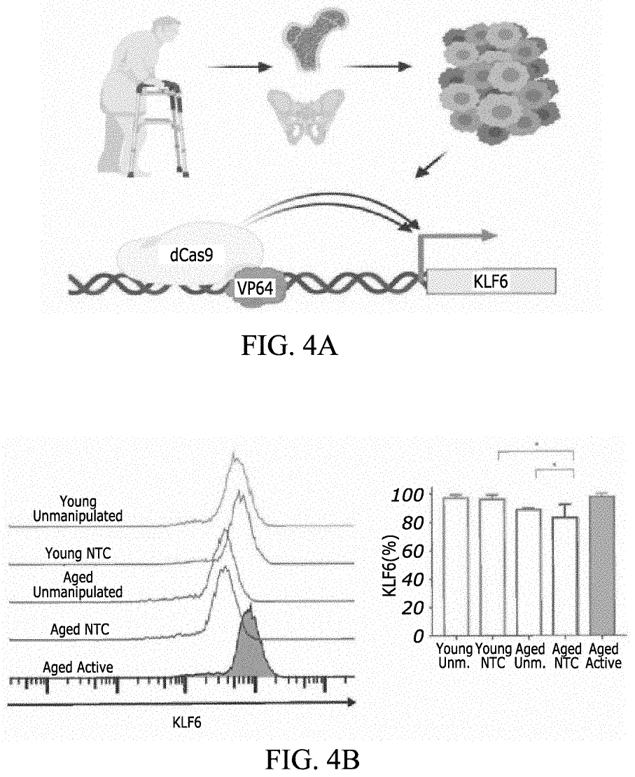

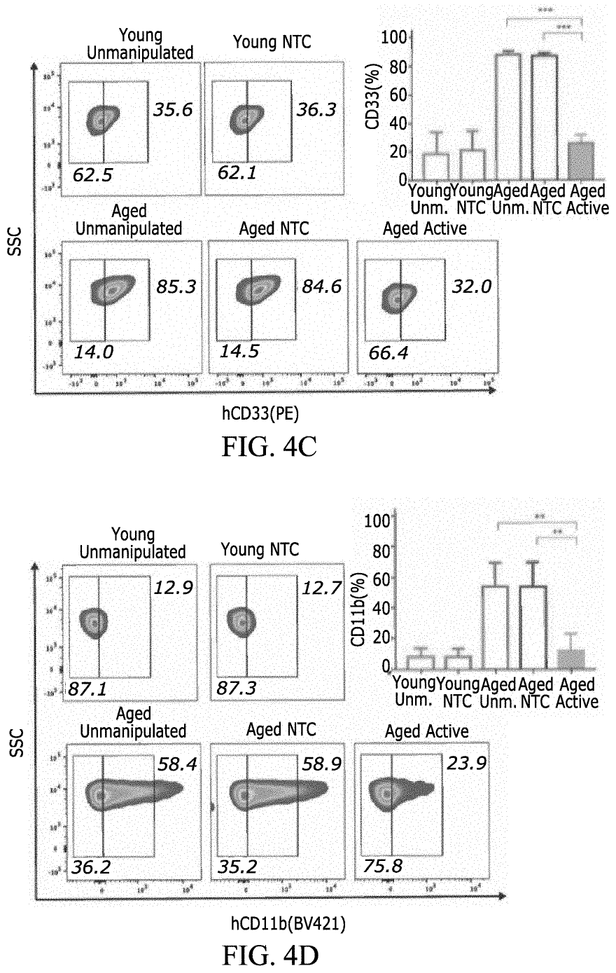

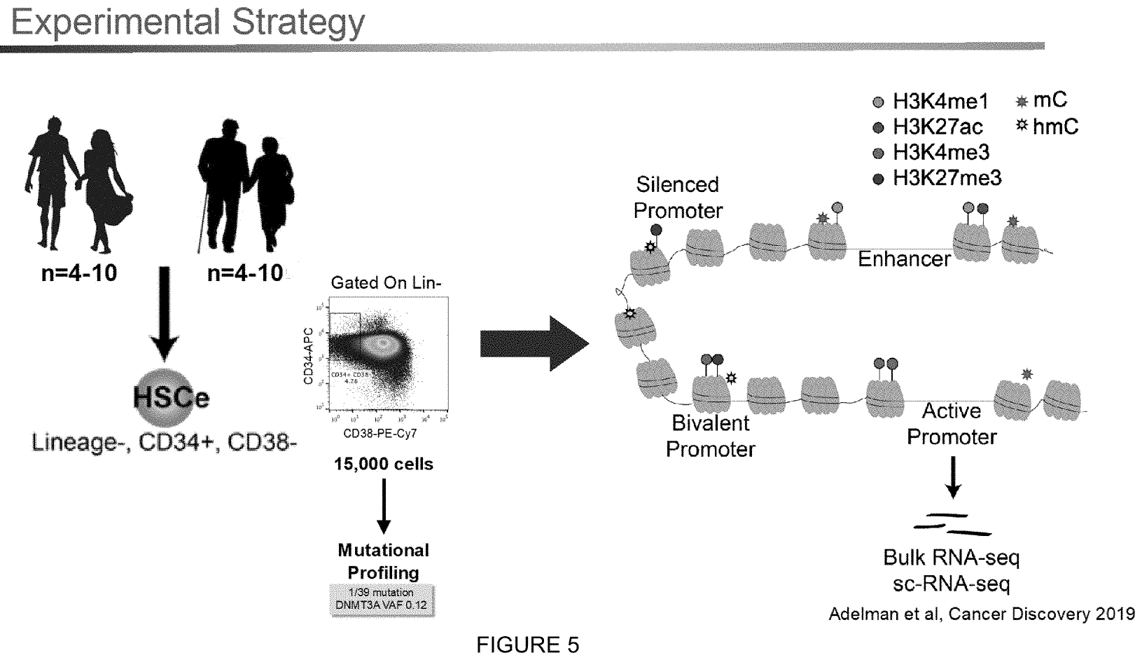

After identifying genes regulated by KLF6-a in aged cells, we then sought to distinguish the core set of KLF6 regulated genes. We found 645 genes that are deregulated with KLF6 KO and have rescued gene expression upon KLF6 activation in aged cells. These genes are associated with ribosomes, immune response, semaphorins, and include a number of other transcription factors and regulators of hematopoiesis, such as GATA1/2, HOXAS/6/7/9, HES1, and MEF2C (FIG. 4H). Importantly, EnrichR pathway analysis showed that the core KLF6 gene signature is associated with platelets and coagulation, adhesion, and myeloid and megakaryocytic differentiation, supporting what we had observed in the liquid culture differentiation assays (FIG. 42E).

Given the transcriptional changes we observed upon reactivation of KLF6 in aged HSPC, we next asked whether KLF6-a is accompanied by epigenetic reprogramming. We performed ChIP-seq for H3K4me1, H3K4me3, and H3K27ac, three activating histone marks we had previously observed are altered with aging [21], in young and aged KLF6-a or NTC CD34+ cells (n=2 biological replicates per condition). While KLF6-a had very no effect on H3K4me3 in either young or aged cells (n=1 differential peaks in aged and n=0 in young, FDR <0.05), there was a marked decrease of H3K4me1 upon KLF6 activation in aged cells (n=3,273 peaks) with relatively few regions with increased H3K4me1 (n=602) (diffbind, FDR <0.05). In contrast, we observed an increase in H3K27ac (n=3,361 peaks) and relatively little loss (n=71 peaks) in aged KLF6-a compared to aged NTC (FDR <0.05). While regions with decreased H3K4me1 in aged HSPC also showed visible, but not significant differences in young KLF6-a cells, no significant differential H3K27ac peaks were identified in young KLF6-a cells compared to NTC, suggesting that the effects of KLF6 activation on this histone modification are indeed age dependent (FIG. 40I).

Genomic annotation showed that regions with H3K27ac loss or increase were mostly distributed throughout promoters, introns, and intergenic regions, with 12% and 49% occurring at active enhancers, respectively. In contrast, the majority of H3K4me1 differential peaks were located at introns and intergenic regions (Figure S5F). In accordance with what we observed at the transcriptional level, regions that gained H3K27ac after KLF6 induction were associated with platelet activation and aggregation, cell junction and cell adhesion as well as coagulation process, whereas the few regions that lost H3K27ac were associated with differentiation of lymphoid progenitor, myeloid, and erythroid cells. Regions with increased H3K4me1 were also associated with coagulation, suggesting it is a key function regulated by KLF6 in aged cells. Moreover, sites with reduced H3K4me1 in aged KLF6-a HSPC were linked to genes involved in granulocyte, neutrophil, B- and T-cell activation, as well as immune signaling, pathways frequently altered with aging (FIG. 40J) (FDR <0.05, Benjamini-Hochberg). To investigate which transcription factors may be contributing to these epigenetic changes, we performed HOMER motif analysis, and found that both regions that gain H3K27ac or lose H3K4me1 are significantly enriched for ERG, ETS1, and FLI1 motifs, suggesting that these are part of a core KLF6 dependent regulatory program (FIG. 40K).

Next, we compared the aged KLF6-a ChIP-seq profiles to those previously generated in aged HSCe, to determine if activation of KLF6 mitigates age-associated loss of H3K4me1 or H3K27ac. We observed an overall trend towards increased H3K27ac in aged, but not young, KLF6-a compared to NTC at regions that normally lose H3K27ac with aging, and found that 8.7% of peaks that gain H3K27ac in aged KLF6-a occur at these age-affected regions, including RXRA, FOXP1,FOSL1, BCOR, SIRT5, KAT6B, SRSF2 and LMNA. In contrast, regions that lose H3K4me1 with HSCe aging tend to have increased H3K4me1 upon KLF6a, both in young and aged HSPC, supporting that indeed, H3K27ac, but not H3K4me1 is specifically influenced by KLF6 in an age-dependent manner (FIG. 42G-H).

KLF6 Induction Reverses Features of HSC Aging In Vivo

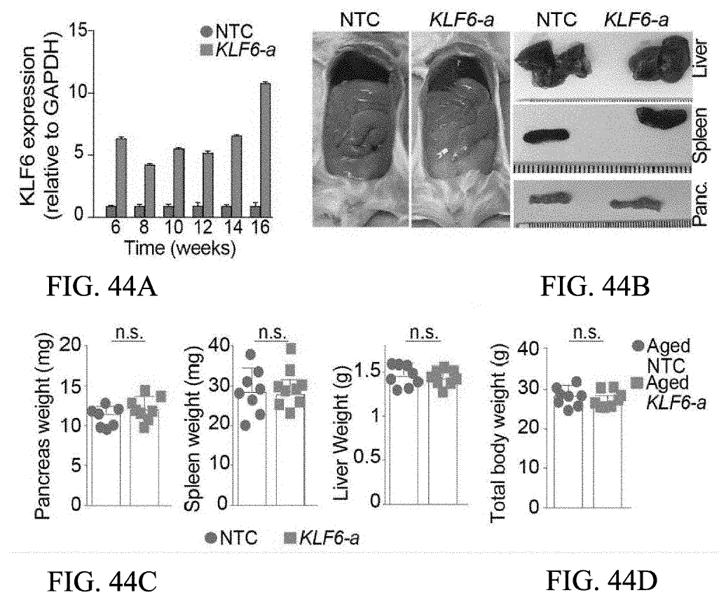

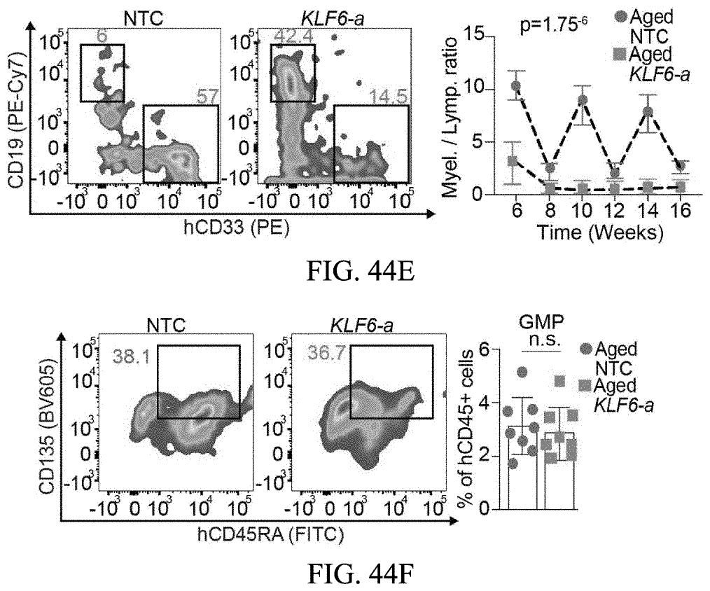

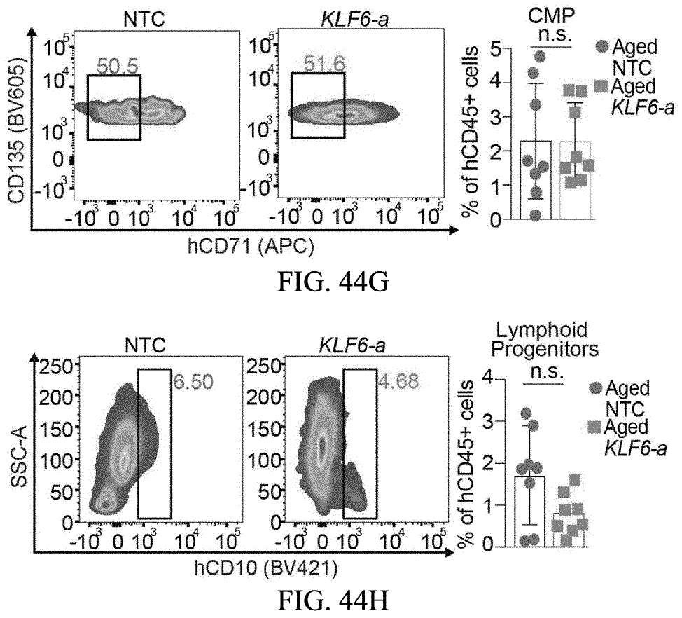

Given the efficiency of KLF6 induction in restoring age-related differentiation defects in vitro, we next tested its ability to reset HSC function in vivo. To test this, we used lentiviral delivery of sgRNAs to stably activate KLF6 (KLF6-a) in human CD34+ cells. Our previously validated sgRNA targeting KLF6 promoter region or NTC sgRNA were cloned into a lenti sgRNA(MS2)_zeo backbone ([46], addgene 61427) and transduced along with dCas9-VP64 (addgene #53192). Following selection, cells were injected into immunodeficient NSG-SGM3 (FIG. 43A). Although previous studies have shown that reprogramming through different strategies can lead to tumor development [47-49] we observed that even after 16 weeks of constant expression (FIG. 44A), KLF6 induction was well tolerated by the animals and did not result in any morphologic abnormalities or malignant transformation (FIG. 44B-D). KLF6 induction in aged CD34+ resulted in a significant reduction in the myeloid output compared to NTC (p<1.2e-8, Linear mixed-effects model), with a concomitant expansion in the lymphoid compartment (p<4.4e-11) and a reduction in the myeloid to lymphoid proportion (p<1.75e-6) (FIG. 43B and FIG. 44E). Analysis of the HSC compartment at week 16 showed a significant reduction in HSC number in KLF6-a cells compared to NTC (p=0.0029) (FIG. 43C). Moreover, KLF6-a resulted in a reduction of the CD34+CD38− and CD34+CD38+ fractions (p=0.0361 and p<0.0001, respectively) (FIG. 43D). In addition, KLF6-a led to a reduction in the MEP population compared to NTC (p=0.041) and a concomitant significant expansion in the multipotent progenitors (p<0.0001) (FIG. 43E-F). Finally, analysis of the GMP, CMP and lymphoid fraction in the bone marrow did not reveal any major changes after KLF6 induction (FIG. 44F-H).

KLF6 Activity Contributes to the Development of Additional HSPC Aging Features

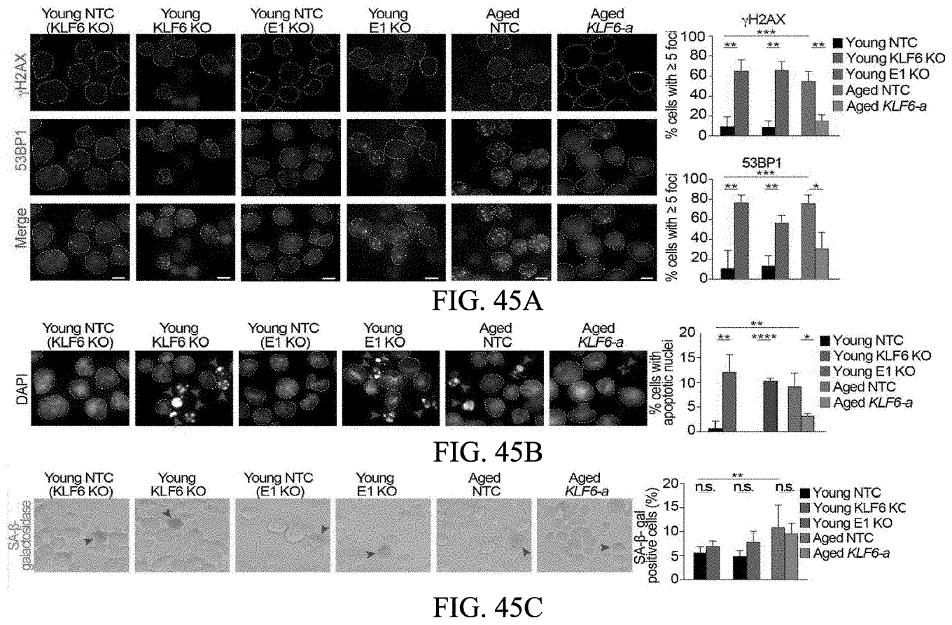

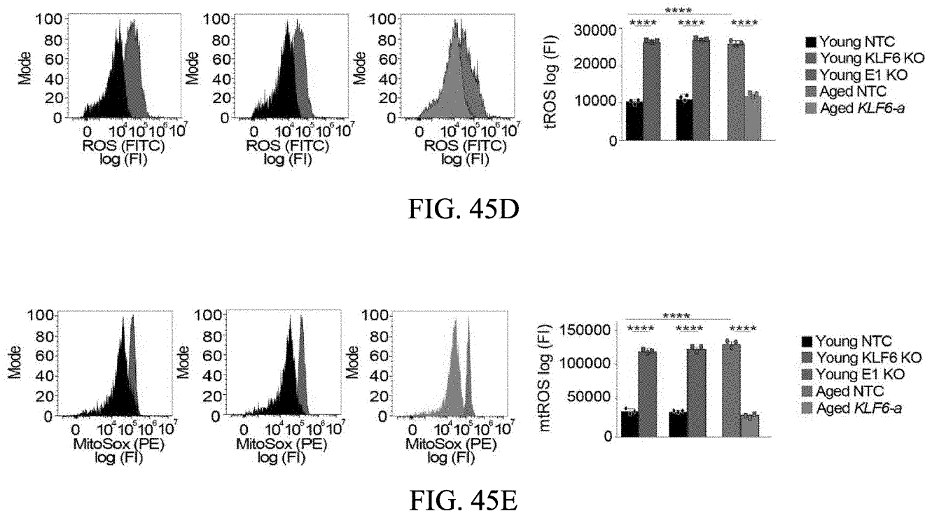

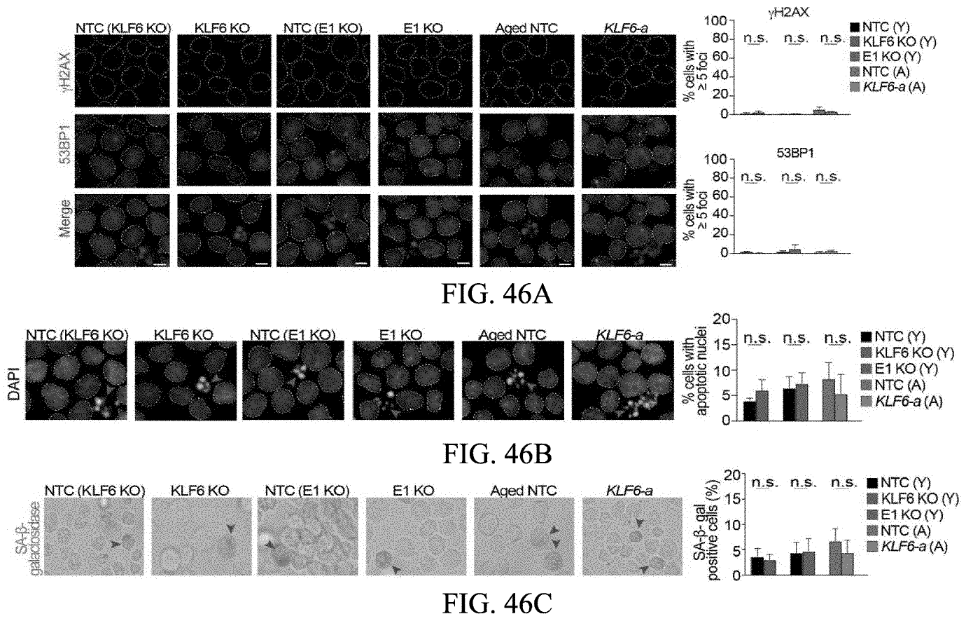

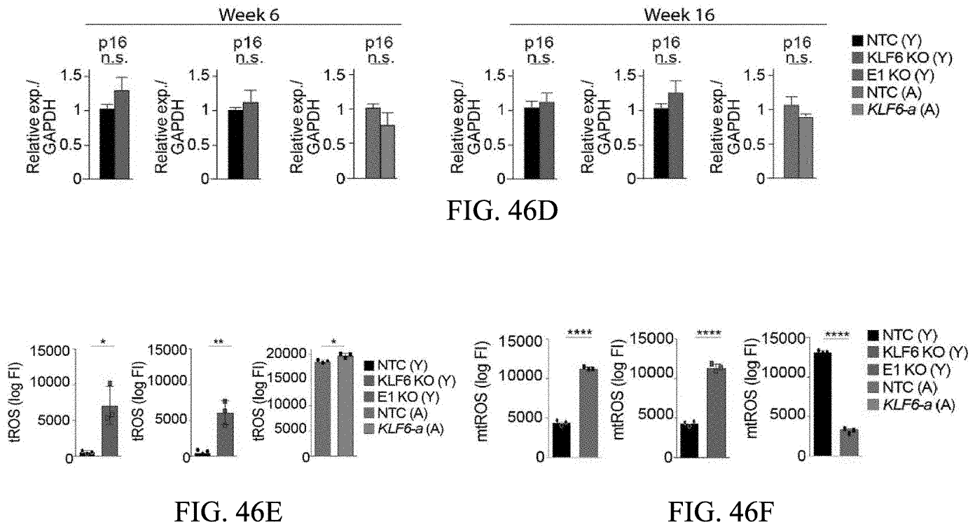

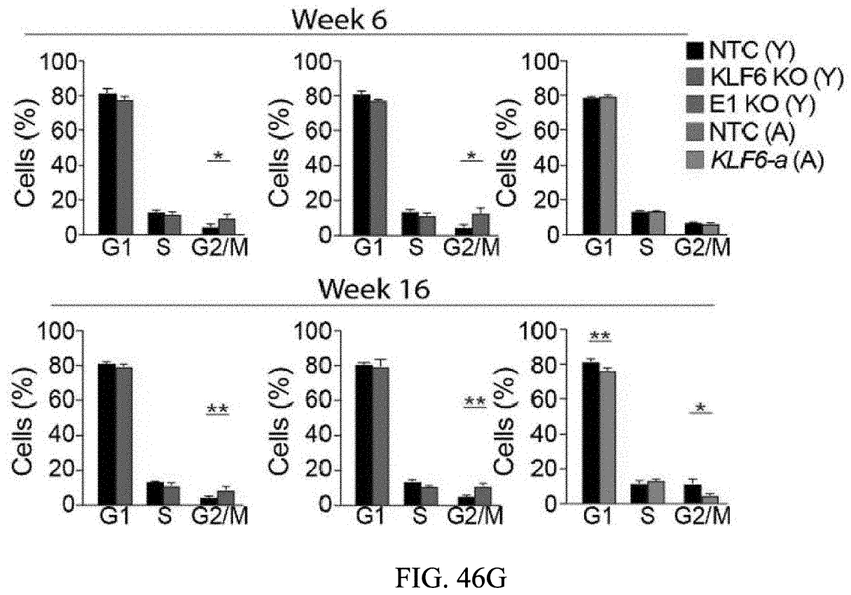

Having validated the role of KLF6 in modulating the expression and epigenetic programs as well as the bone marrow and peripheral blood composition, we next sought to expand these observations by analyzing the impact of KLF6 modulation on additional features of aging. Previous studies have shown that the number of DNA-damage foci accumulates in most tissues during aging [10, 13]. Using the S139 phosphorylated histone H2AX (γH2AX) and 53BP1 as double-strand break (DSB) markers, we first observed that baseline levels of these DBS markers were higher in age NTC in comparison with young controls (p=0.0001 and p>0.0001, respectively) (FIG. 45A). We then tested how KLF6 modulation could affect these DSB markers and observed that young human KLF6 KO or E1 KO cells did not display major differences in comparison to NTC cells 6 weeks post-injection into immunodeficient mice (FIG. 46A). However, when we analyzed these features at 16 weeks, we detected a significant increase in [symbol] H2AX and 53BP1 in both KLF6 KO or E1 KO cells (p>0.0001) (FIG. 45A), suggesting that KLF6 disruption contributes to DSB accumulation over time. Notably, induction of KLF6 in aged human cells reduced the number of γH2AX and 53BP1 foci in comparison with aged NTC cells, as measured at 16 weeks PI (p>0.0001) (FIG. 45A and FIG. 46A).

As increased apoptosis is common feature of HSC aging [10, 12], we next assessed the reference apoptotic levels during normal aging and detected that younger cells displayed a lower apoptotic frequency than aged NTC cells (p=0.008) (FIG. 45B). In addition, we were able to detect that rate of apoptotic levels and found that KLF6 KO or E1 KO cells at a late, but not early timepoint during reconstitution, contributed to an enhanced apoptotic rate in comparison with NTC cells (p=0.0061 and p>0.0001, respectively) (FIG. 45B and FIG. 46B). We then asked whether KLF6 induction in aged HSC could be sufficient to reverse the apoptotic levels observed in an aged HSC context. Indeed, we were able to detect that at 16, but not 6 weeks PI, KLF6 induced aged HSPCs exhibit a reduction in the incidence of apoptotic cells (p=0.0188) (FIG. 45B and FIG. 46B). Although we were able to distinguish that aged controls display higher levels of senescence-associated b-galactosidase (SA-13-Gal) than young controls at baseline, no major distinctions with regard to senescence related makers such as SA-β-Gal or p16 expression, were observed after KLF6 KO, E1 KO or KLF6 induction (FIG. 45C, FIG. 46C-D).

Since oxidative stress and mitochondrial dysfunction are common features of aging and KLF6 has been implicated as a major mitochondrial regulator [50-54], we wondered whether these functions were also impaired in young human CD34+ cells after KLF6 or E1 depletion. After validating that aged controls displayed higher baseline levels of total and mitochondrial reactive oxygen species (tROS and mtROS, respectively) levels than young controls (p<0.0001, for both), we then found that disruption of KLF6 or E1 region results in increased levels of total reactive oxygen species (tROS) at week 16 (p=0.0008 and p=0.0054, respectively) (FIG. 45D and FIG. 46E). Furthermore, this feature appears to be rescued upon KLF6 induction in aged cells upon challenge with xenotransplantation (p=0.0002) (FIG. 45D and FIG. 46E). Since approximately 90% of cellular ROS are generated in the mitochondria [55], we next evaluated mitochondrial ROS (mtROS) from KLF6 KO or E1 KO cells and observed a significant increase in the mtROS levels generated after KLF6 or E1 disruption at 6 (p<0.0001 and p=0.0009, respectively) (FIG. 46F) as well as 16 weeks PI (p=0.0001 and p=0.0005, respectively) (FIG. 44E), suggesting that the tROS increase observed, is a consequence of mtROS accumulation after KLF6 depletion. Conversely, mtROS assessment in an aged context after KLF6 induction revealed a significant decline in the mtROS levels at both, 6- and 16-weeks PI (p<0.0001) (FIG. 45E and FIG. 46F).

HSC cell cycle regulation is carefully controlled by a complex interaction between cell intrinsic and extrinsic regulatory network that becomes altered with age. Previous studies have found a wide spectrum of findings regarding aged HSCs proliferation [10, 13, 56]. Cell cycle distribution revealed an increased frequency of cells in G2/M phase of the cell cycle at 6- and 16-weeks after KLF6 (p≤0.0294 and p=0.0064, respectively) or E1 depletion in young HSC (p≤0.0125 and p=0.0034, respectively) whereas induction of KLF6 in aged cells resulted in a decreased proportion of these cells, compared to non-activated aged controls (p=0.0120) (FIG. 46G). Furthermore, after KLF6 induction in aged HSPC, we noticed a reduced proportion of cells circulating through the G1 phase of the cell cycle 16 weeks PI (p=0.0094) (FIG. 46G), consistent with previous reports that found it to be increased in aged normal donors [10]. Together these results provide evidence that KLF6 is critical in modulating normal HSPC function and that loss of KLF6 results in accumulation of both tROS and mtROS, which triggers DNA damage accumulation compromising HSPC dynamics over time. Moreover, we observed that restoring KLF6 function reverses tROS and mtROS production, which in turn leads to a decrease in the frequency of DSB in an aged context.

Discussion

Aging is a well-established risk factor for most disorders in contemporary societies [57]. Still, the intricacy of cellular aging has limited our knowledge of this critical biological process. In the hematopoietic system, aging is associated with abnormalities in the HSC compartment including changes in clonal composition and lineage contribution. The hematopoietic system relies on a reduced fraction of HSCs resident in the bone marrow to generate ˜1011 new cells every day. HSCs have the ability to self-renew and differentiate through a complex cascade of gradually committed and lineage restricted progenitors to finally generate all mature circulating myeloid and lymphoid cell types [58]. In this context, epigenetic deregulation has emerged as one of the hallmarks of aging [21, 59, 60]. Likewise, cellular reprogramming to pluripotency follows a widespread epigenetic remodeling [59, 61, 62]. Previous studies into cellular rejuvenation have implicated epigenetic reprogramming [61, 63-65] but this process remains poorly understood in human HSC.

Having previously found evidence that HSC aging results in epigenomic reprogramming and that KLF6 is one of the top deregulated genes with age [21, 34, 66], here we demonstrate that KLF6 is an essential factor, required for normal HSC commitment and function. Aged HSC are characterized by increased self-renewal and diminished homing capacity, reduced lymphoid output producing a myeloid differentiation predisposition as well as an increased risk of malignant transformation [5-8]. Here, to the best of our knowledge, we demonstrate that KLF6-deprived HSC display a preferential reconstitution increase, both in vitro and in vivo, of the myeloid lineage over that of the lymphoid. Moreover, we observed that KLF6-depleted HSPCs exhibit an in vivo expansion of the HSC and progenitor compartments and, at the transcriptional level, display aging-like profiles as well as a strong enrichment of several leukemias associated gene signatures. Together, these results show that KLF6 is a fundamental regulator of HSPC biology and is required for normal HSPC function and commitment.

While It has been previously demonstrated that successfully reprogrammed cells can lead to cancer development and teratomas formation in vivo [47, 49], others have shown that ectopic administration of reprograming factors can be achieved safely short and long-term, without the occurrence of neoplastic lesions or tumor formation [48, 67, 68]. Our studies demonstrate that KLF6 induction in an aged normal context has the ability to reprogram and rejuvenate HSC by reversing key hallmarks of aging while avoiding malignant transformation.

Hematopoietic cell transplantation (HCT) remains a potentially curative treatment for life-threatening hematological and non-hematological disorders and donor and recipient ages are key aspects that impact transplantation outcomes. Over the last years, the total number of HCTs completed worldwide has exceeded 60,000 a year [69, 70]. Nearly 70% of allogeneic HSC (allo-HSC) transplants use hematopoietic progenitor cells (HPCs) from unrelated donors with a permanent annual increase of about 10% [69, 71]. Aged HSCs, while increasing in number, display a decline in functionality in comparison to their younger counterparts [6, 8, 9]. In the clinical setting this translates to inferior outcomes in HCTs from older donors, with lower engraftment levels and increased transplant related mortality [72, 73]. The recent development of CRISPR-based technologies along with our observations of the role of KLF6 in modulating features of aging in HSC opens the door for the possibility of ex-vivo reprogramming of aged HSC prior to re-infusion as a mode of enhancing engraftment from aged donors, thus expanding the available donor pool. Epigenome editing therapeutic approaches provide the novelty of engineering cells to increase their potential and reset their functions without resulting in permanent sequence changes to the genome. This may prove to be helpful not only in the context of HCT donors, but also in the context of physiological human aging, where HSC reprogramming may help improve clinically relevant cytopenias secondary to HSC dysfunction.

This study provides a proof of concept for developing a new approach for HSC rejuvenation. Our observations demonstrate that reprogramming through KLF6 can ameliorate HSPC aging and set the ground to better recognize the molecular mechanisms behind normal biological aging. We speculate that KLF6 may regulate stem cells in other tissues, since this is a ubiquitously expressed factor. Further studies will determine the effect of KLF6 on lifespan. These results will also be necessary if we are to develop efficient rejuvenation strategies towards maximizing the beneficial effects of age reprogramming while avoiding potential risks associated with the expression of other reprogramming strategies in vivo.

Sequences:

UbC-dCas9 VP64-T2A-GFP (addgene #53192):
(SEQ ID NO: 21)
GTCGACGGATCGGGAGATCTCCCGATCCCCTATGGTGCACTCTCAGTACAATCTGCTCTGATGCCGCATAGTTAA
GCCAGTATCTGCTCCCTGCTTGTGTGTTGGAGGTCGCTGAGTAGTGCGCGAGCAAAATTTAAGCTACAACAAGGCAAGGCTT
GACCGACAATTGCATGAAGAATCTGCTTAGGGTTAGGCGTTTTGCGCTGCTTCGCGATGTACGGGCCAGATATACGCGTTGA
CATTGATTATTGACTAGTTATTAATAGTAATCAATTACGGGGTCATTAGTTCATAGCCCATATATGGAGTTCCGCGTTACATAA
CTTACGGTAAATGGCCCGCCTGGCTGACCGCCCAACGACCCCCGCCCATTGACGTCAATAATGACGTATGTTCCCATAGTAA
CGCCAATAGGGACTTTCCATTGACGTCAATGGGTGGAGTATTTACGGTAAACTGCCCACTTGGCAGTACATCAAGTGTATCA
TATGCCAAGTACGCCCCCTATTGACGTCAATGACGGTAAATGGCCCGCCTGGCATTATGCCCAGTACATGACCTTATGGGAC
TTTCCTACTTGGCAGTACATCTACGTATTAGTCATCGCTATTACCATGGTGATGCGGTTTTGGCAGTACATCAATGGGCGTGG
ATAGCGGTTTGACTCACGGGGATTTCCAAGTCTCCACCCCATTGACGTCAATGGGAGTTTGTTTTGGCACCAAAATCAACGG
GACTTTCCAAAATGTCGTAACAACTCCGCCCCATTGACGCAAATGGGCGGTAGGCGTGTACGGTGGGAGGTCTATATAAGC
AGCGCGTTTTGCCTGTACTGGGTCTCTCTGGTTAGACCAGATCTGAGCCTGGGAGCTCTCTGGCTAACTAGGGAACCCACT
GCTTAAGCCTCAATAAAGCTTGCCTTGAGTGCTTCAAGTAGTGTGTGCCCGTCTGTTGTGTGACTCTGGTAACTAGAGATCC
CTCAGACCCTTTTAGTCAGTGTGGAAAATCTCTAGCAGTGGCGCCCGAACAGGGACTTGAAAGCGAAAGGGAAACCAGAGG
AGCTCTCTCGACGCAGGACTCGGCTTGCTGAAGCGCGCACGGCAAGAGGCGAGGGGCGGCGACTGGTGAGTACGCCAAA
AATTTTGACTAGCGGAGGCTAGAAGGAGAGAGATGGGTGCGAGAGCGTCAGTATTAAGCGGGGGAGAATTAGATCGCGATG
GGAAAAAATTCGGTTAAGGCCAGGGGGAAAGAAAAAATATAAATTAAAACATATAGTATGGGCAAGCAGGGAGCTAGAACGA
TTCGCAGTTAATCCTGGCCTGTTAGAAACATCAGAAGGCTGTAGACAAATACTGGGACAGCTACAACCATCCCTTCAGACAG
GATCAGAAGAACTTAGATCATTATATAATACAGTAGCAACCCTCTATTGTGTGCATCAAAGGATAGAGATAAAAGACACCAAG
GAAGCTTTAGACAAGATAGAGGAAGAGCAAAACAAAAGTAAGACCACCGCACAGCAAGCGGCCGCTGATCTTCAGACCTGG
AGGAGGAGATATGAGGGACAATTGGAGAAGTGAATTATATAAATATAAAGTAGTAAAAATTGAACCATTAGGAGTAGCCCCAC
CAAGGCAAAGAGAAGAGTGGTGCAGAGAGAAAAAAGAGCAGTGGGAATAGGAGCTTTGTTCCTTGGGTTCTTGGGAGCAGC
AGGAAGCACTATGGGCGCAGCGTCAATGACGCTGACGGTACAGGCCAGACAATTATTGTCTGGTATAGTGCAGCAGCAGAA
CAATTTGCTGAGGGCTATTGAGGCGCAACAGCATCTGTTGCAACTCACAGTCTGGGGCATCAAGCAGCTCCAGGCAAGAAT
CCTGGCTGTGGAAAGATACCTAAAGGATCAACAGCTCCTGGGGATTTGGGGTTGCTCTGGAAAACTCATTTGCACCACTGCT
GTGCCTTGGAATGCTAGTTGGAGTAATAAATCTCTGGAACAGATTTGGAATCACACGACCTGGATGGAGTGGGACAGAGAAA
TTAACAATTACACAAGCTTAATACACTCCTTAATTGAAGAATCGCAAAACCAGCAAGAAAAGAATGAACAAGAATTATTGGAAT
TAGATAAATGGGCAAGTTTGTGGAATTGGTTTAACATAACAAATTGGCTGTGGTATATAAAATTATTCATAATGATAGTAGGAG
GCTTGGTAGGTTTAAGAATAGTTTTTGCTGTACTTTCTATAGTGAATAGAGTTAGGCAGGGATATTCACCATTATCGTTTCAGA
CCCACCTCCCAACCCCGAGGGGACCCGACAGGCCCGAAGGAATAGAAGAAGAAGGTGGAGAGAGAGACAGAGACAGATCC
ATTCGATTAGTGAACGGATCGGCACTGCGTGCGCCAATTCTGCAGACAAATGGCAGTATTCATCCACAATTTTAAAAGAAAAG
GGGGGATTGGGGGGTACAGTGCAGGGGAAAGAATAGTAGACATAATAGCAACAGACATACAAACTAAAGAATTACAAAAACA
AATTACAAAAATTCAAAATTTTCGGGTTTATTACAGGGACAGCAGAGATCCAGTTTGGTTAatTAAATAACTTCGTATAGCATAC
ATTATACGAAGTTATGATAAGAGACGGTGGTGgcgccgctacagggcgcgtcccattcgccattcaggctgcgcaactgttgggaagggcgatcggtgcgggc
ctcttcgctattacgccagctggcgaaagggggatgtgctgcaaggcgattaagttgggtaacgccagggttttcccagtcacgacgttgtaaaacgacggcc
agtgagcgcgcgtaatacgactcactatagggcgaattgggtaccgggccccccctcgaggtcctccagcttttgttccctttagtgagggttaattgcgcgc
ttggcgtaatcatggtcatagctgtttcctgtgtgaaattgttatccgctcacaattccacacaacatacgagccggaagcataaagtgtaaagcctggggtg
cctaatgagtgagctaactcacattaattgcgttgcgctcactgcccgctttccaCTGCATGACGTCTCCACAATTAatTAAgggtgcagcggcctccgcgcc
gggttttggcgcctcccgcgggcgcccccctcctcacggcgagcgctgccacgtcagacgaagggcgcaggagcgttcctgatccttccgcccggacgctcag
gacagcggcccgctgctcataagactcggccttagaaccccagtatcagcagaaggacattttaggacgggacttgggtgactctagggcactggttttcttt
ccagagagcggaacaggcgaggaaaagtagtcccttctcggcgattctgcggagggatctccgtggggcggtgaacgccgatgattatataaggacgcgccgg
gtgtggcacagctagttccgtcgcagccgggatttgggtcgcggttcttgtttgtggatcgctgtgatcgtcacttggtgagttgcgggctgctgggctggcc
ggggctttcgtggccgccgggccgctcggtgggacggaagcgtgtggagagaccgccaagggctgtagtctgggtccgcgagcaaggttgccctgaactgggg
gttggggggagcgcacaaaatggcggctgttcccgagtcttgaatggaagacgcttgtaaggcgggctgtgaggtcgttgaaacaaggtggggggcatggtgg
gcggcaagaacccaaggtcttgaggccttcgctaatgcgggaaagctcttattcgggtgagatgggctggggcaccatctggggaccctgacgtgaagtttgt
cactgactggagaactcgggtttgtcgtctggttgcgggggcggcagttatgcggtgccgttgggcagtgcacccgtacctttgggagcgcgcgcctcgtcgt
gtcgtgacgtcacccgttctgttggcttataatgcagggtggggccacctgccggtaggtgtgcggtaggcttttctccgtcgcaggacgcagggttcgggcc
tagggtaggctctcctgaatcgacaggcgccggacctctggtgaggggagggataagtgaggcgtcagtttctttggtcggttttatgtacctatcttcttaa
gtagctgaagctccggttttgaactatgcgctcggggttggcgagtgtgttttgtgaagttttttaggcaccttttgaaatgtaatcatttgggtcaatatgt
aattttcagtgttagactagTaaattgtccgctaaattctggccgtttttggcttttttgttagacGAAGCTTGGGCTGCAGGTCGACTctagAgccaccATG
GACTACAAAGACCATGACGGTGATTATAAAGATCATGACATCGATTACAAGGATGACGATGACAAGatggcccccaagaagaagaggaaggtgggccgc
ggaATGGACAAGAAGTACTCCATTGGGCTCGCCATCGGCACAAACAGCGTCGGCTGGGCCGTCATTACGGACGAGTACAAG
GTGCCGAGCAAAAAATTCAAAGTTCTGGGCAATACCGATCGCCACAGCATAAAGAAGAACCTCATTGGCGCCCTCCTGTTCG
ACTCCGGGGAAACCGCCGAAGCCACGCGGCTCAAAAGAACAGCACGGCGCAGATATACCCGCAGAAAGAATCGGATCTGC
TACCtgcaGGAGATCTTTAGTAATGAGATGGCTAAGGTGGATGACTCTTTCTTCCATAGGCTGGAGGAGTCCTTTTTGGTGGAG
GAGGATAAAAAGCACGAGCGCCACCCAATCTTTGGCAATATCGTGGACGAGGTGGCGTACCATGAAAAGTACCCAACCATAT
ATCATCTGAGGAAGAAGCTTGTAGACAGTACTGATAAGGCTGACTTGCGGTTGATCTATCTCGCGCTGGCGCATATGATCAA
ATTTCGGGGACACTTCCTCATCGAGGGGGACCTGAACCCAGACAACAGCGATGTCGACAAACTCTTTATCCAACTGGTTCAG
ACTTACAATCAGCTTTTCGAAGAGAACCCGATCAACGCATCCGGAGTTGACGCCAAAGCAATCCTGAGCGCTAGGCTGTCCA
AATCCCGGCGGCTCGAAAACCTCATCGCACAGCTCCCTGGGGAGAAGAAGAACGGCCTGTTTGGTAATCTTATCGCCCTGT
CACTCGGGCTGACCCCCAACTTTAAATCTAACTTCGACCTGGCCGAAGATGCCAAGCTTCAACTGAGCAAAGACACCTACGA
TGATGATCTCGACAATCTGCTGGCCCAGATCGGCGACCAGTACGCAGACCTTTTTTTGGCGGCAAAGAACCTGTCAGACGC
CATTCTGCTGAGTGATATTCTGCGAGTGAACACGGAGATCACCAAAGCTCCGCTGAGCGCTAGTATGATCAAGCGCTATGAT
GAGCACCACCAAGACTTGACTTTGCTGAAGGCCCTTGTCAGACAGCAACTGCCTGAGAAGTACAAGGAAATTTTCTTCGATC
AGTCTAAAAATGGCTACGCCGGATACATTGACGGCGGAGCAAGCCAGGAGGAATTTTACAAATTTATTAAGCCCATCTTGGA
AAAAATGGACGGCACCGAGGAGCTGCTGGTAAAGCTTAACAGAGAAGATCTGTTGCGCAAACAGCGCACTTTCGACAATGG
AAGCATCCCCCACCAGATTCACCTGGGCGAACTGCACGCTATCCTCAGGCGGCAAGAGGATTTCTACCCCTTTTTGAAAGAT
AACAGGGAAAAGATTGAGAAAATCCTCACATTTCGGATACCCTACTATGTAGGCCCCCTCGCCCGGGGAAATTCCAGATTCG
CGTGGATGACTCGCAAATCAGAAGAGACCATCACTCCCTGGAACTTCGAGGAAGTCGTGGATAAGGGGGCCTCTGCCCAGT
CCTTCATCGAAAGGATGACTAACTTTGATAAAAATCTGCCTAACGAAAAGGTGCTTCCTAAACACTCTCTGCTGTACGAGTAC
TTCACAGTTTATAACGAGCTCACCAAGGTCAAATACGTCACAGAAGGGATGAGAAAGCCAGCATTCCTGTCTGGAGAGCAGA
AGAAAGCTATCGTGGACCTCCTCTTCAAGACGAACCGGAAAGTTACCGTGAAACAGCTCAAAGAAGACTATTTCAAAAAGATT
GAATGTTTCGACTCTGTTGAAATCAGCGGAGTGGAGGATCGCTTCAACGCATCCCTGGGAACGTATCACGATCTCCTGAAAA
TCATTAAAGACAAGGACTTCCTGGACAATGAGGAGAACGAGGACATTCTTGAGGACATTGTCCTCACCCTTACGTTGTTTGAA
GATAGGGAGATGATTGAAGAACGCTTGAAAACTTACGCTCATCTCTTCGACGACAAAGTCATGAAACAGCTCAAGAGGCGCC
GATATACAGGATGGGGGCGGCTGTCAAGAAAACTGATCAATGGgatcCGAGACAAGCAGAGTGGAAAGACAATCCTGGATTTT
CTTAAGTCCGATGGATTTGCCAACCGGAACTTCATGCAGTTGATCCATGATGACTCTCTCACCTTTAAGGAGGACATCCAGAA
AGCACAAGTTTCTGGCCAGGGGGACAGTCTTCACGAGCACATCGCTAATCTTGCAGGTAGCCCAGCTATCAAAAAGGGAATA
CTGCAGACCGTTAAGGTCGTGGATGAACTCGTCAAAGTAATGGGAAGGCATAAGCCCGAGAATATCGTTATCGAGATGGCC
CGAGAGAACCAAACTACCCAGAAGGGACAGAAGAACAGTAGGGAAAGGATGAAGAGGATTGAAGAGGGTATAAAAGAACTG
GGGTCCCAAATCCTTAAGGAACACCCAGTTGAAAACACCCAGCTTCAGAATGAGAAGCTCTACCTGTACTACCTGCAGAACG
GCAGGGACATGTACGTGGATCAGGAACTGGACATCAATCGGCTCTCCGACTACGACGTGGATGCCATCGTGCCCCAGTCTT
TTCTCAAAGATGATTCTATTGATAATAAAGTGTTGACAAGATCCGATAAAAATAGAGGGAAGAGTGATAACGTCCCCTCAGAA
GAAGTTGTCAAGAAAATGAAAAATTATTGGCGGCAGCTGCTGAACGCCAAACTGATCACACAACGGAAGTTCGATAATCTGA
CTAAGGCTGAACGAGGTGGCCTGTCTGAGTTGGATAAAGCCGGCTTCATCAAAAGGCAGCTTGTTGAGACACGCCAGATCA
CCAAgcacGTGGCCCAAATTCTCGATTCACGCATGAACACCAAGTACGATGAAAATGACAAACTGATTCGAGAGGTGAAAGTT
ATTACTCTGAAGTCTAAGCTGGTCTCAGATTTCAGAAAGGACTTTCAGTTTTATAAGGTGAGAGAGATCAACAATTACCACCAT
GCGCATGATGCCTACCTGAATGCAGTGGTAGGCACTGCACTTATCAAAAAATATCCCAAGCTTGAATCTGAATTTGTTTACGG
AGACTATAAAGTGTACGATGTTAGGAAAATGATCGCAAAGTCTGAGCAGGAAATAGGCAAGGCCACCGCTAAGTACTTCTTTT
ACAGCAATATTATGAATTTTTTCAAGACCGAGATTACACTGGCCAATGGAGAGATTCGGAAGCGACCACTTATCGAAACAAAC
GGAGAAACAGGAGAAATCGTGTGGGACAAGGGTAGGGATTTCGCGACAGTCCGGAAGGTCCTGTCCATGCCGCAGGTGAA
CATCGTTAAAAAGACCGAAGTACAGACCGGAGGCTTCTCCAAGGAAAGTATCCTCCCGAAAAGGAACAGCGACAAGCTGAT
CGCACGCAAAAAAGATTGGGACCCCAAGAAATACGGCGGATTCGATTCTCCTACAGTCGCTTACAGTGTACTGGTTGTGGCC
AAAGTGGAGAAAGGGAAGTCTAAAAAACTCAAAAGCGTCAAGGAACTGCTGGGCATCACAATCATGGAGCGATCAAGCTTCG
AAAAAAACCCCATCGACTTTCTCGAGGCGAAAGGATATAAAGAGGTCAAAAAAGACCTCATCATTAAGCTTCCCAAGTACTCT
CTCTTTGAGCTTGAAAACGGCCGGAAACGAATGCTCGCTAGTGCGGGCGAGCTGCAGAAAGGTAACGAGCTGGCACTGCC
CTCTAAATACGTTAATTTCTTGTATCTGGCCAGCCACTATGAAAAGCTCAAAGGGTCTCCCGAAGATAATGAGCAGAAGCAGC
TGTTCGTGGAACAACACAAACACTACCTTGATGAGATCATCGAGCAAATAAGCGAATTCTCCAAAAGAGTGATCCTCGCCGA
CGCTAACCTCGATAAGGTGCTTTCTGCTTACAATAAGCACAGGGATAAGCCCATCAGGGAGCAGGCAGAAAACATTATCCAC
TTGTTTACTCTGACCAACTTGGGCGCGCCTGCAGCCTTCAAGTACTTCGACACCACCATAGACAGAAAGCGGTACACCTCTA
CAAAGGAGGTCCTGGACGCCACACTGATTCATCAGTCAATTACGGGGCTCTATGAAACAAGAATCGACCTCTCTCAGCTCGG
TGGAGACAGCAGGGCTGACCCCAAGAAGAAGAGGAAGGTGGctagCCGCGCCGACGCGCTGGACGATTTCGATCTCGACAT
GCTGGGTTCTGATGCCCTCGATGACTTTGACCTGGATATGTTGGGAAGCGACGCATTGGATGACTTTGATTGGACATGCTCG
GCTCCGATGCTCTGGACGATTTCGATCTCGATATGTTAATCGctagCGAGGGCAGAGGAAGTCTTCTAACATGCGGTGACGTG
GAGGAGAATCCCGGCCCTGgtacCgtgagcaagggcgaggagctgttcaccggggtggtgcccatcctggtcgagctggacggcgacgtaaacggccacaagt
tcagcgtgtccggcgagggcgagggcgatgccacctacggcaagctgaccctgaagttcatctgcaccaccggcaagctgcccgtgccctggcccaccctcgt
gaccaccctgacctacggcgtgcagtgcttcagccgctaccccgaccacatgaagcagcacgacttcttcaagtccgccatgcccgaaggctacgtccaggag
cgcaccatcttcttcaaggacgacggcaactacaagacccgcgccgaggtgaagttcgagggcgacaccctggtgaaccgcatcgagctgaagggcatcgact
tcaaggaggacggcaacatcctggggcacaagctggagtacaactacaacagccacaacgtctatatcatggccgacaagcagaagaacggcatcaaggtgaa
cttcaagatccgccacaacatcgaggacggcagcgtgcagctcgccgaccactaccagcagaacacccccatcggcgacggccccgtgctgctgcccgacaac
cactacctgagcacccagtccgccctgagcaaagaccccaacgagaagcgcgatcacatggtcctgctggagttcgtgaccgccgccgggatcactctcggca
tggacgagctgtacaagAccggTTGATAATAGATAACTTCGTATAGCATACATTATACG
AAGTTATGaattCGATATCAAGCTTATCGATAATCAACCTCTGGATTACAAAATTTGTGAAAGATTGACTGGTATTCTTAACTATG
TTGCTCCTTTTACGCTATGTGGATACGCTGCTTTAATGCCTTTGTATCATGCTATTGCTTCCCGTATGGCTTTCATTTTCTCCT
CCTTGTATAAATCCTGGTTGCTGTCTCTTTATGAGGAGTTGTGGCCCGTTGTCAGGCAACGTGGCGTGGTGTGCACTGTGTT
TGCTGACGCAACCCCCACTGGTTGGGGCATTGCCACCACCTGTCAGCTCCTTTCCGGGACTTTCGCTTTCCCCCTCCCTATT
GCCACGGCGGAACTCATCGCCGCCTGCCTTGCCCGCTGCTGGACAGGGGCTCGGCTGTTGGGCACTGACAATTCCGTGGT
GTTGTCGGGGAAATCATCGTCCTTTCCTTGGCTGCTCGCCTGTGTTGCCACCTGGATTCTGCGCGGGACGTCCTTCTGCTAC
GTCCCTTCGGCCCTCAATCCAGCGGACCTTCCTTCCCGCGGCCTGCTGCCGGCTCTGCGGCCTCTTCCGCGTCTTCGCCTT
CGCCCTCAGACGAGTCGGATCTCCCTTTGGGCCGCCTCCCCGCATCGATACCGTCGACCTCGAGACCTAGAAAAACATGGA
GCAATCACAAGTAGCAATACAGCAGCTACCAATGCTGATTGTGCCTGGCTAGAAGCACAAGAGGAGGAGGAGGTGGGTTTT
CCAGTCACACCTCAGGTACCTTTAAGACCAATGACTTACAAGGCAGCTGTAGATCTTAGCCACTTTTTAAAAGAAAAGGGGG
GACTGGAAGGGCTAATTCACTCCCAACGAAGACAAGATATCCTTGATCTGTGGATCTACCACACACAAGGCTACTTCCCTGA
TTGGCAGAACTACACACCAGGGCCAGGGATCAGATATCCACTGACCTTTGGATGGTGCTACAAGCTAGTACCAGTTGAGCAA
GAGAAGGTAGAAGAAGCCAATGAAGGAGAGAACACCCGCTTGTTACACCCTGTGAGCCTGCATGGGATGGATGACCCGGA
GAGAGAAGTATTAGAGTGGAGGTTTGACAGCCGCCTAGCATTTCATCACATGGCCCGAGAGCTGCATCCGGACTGTACTGG
GTCTCTCTGGTTAGACCAGATCTGAGCCTGGGAGCTCTCTGGCTAACTAGGGAACCCACTGCTTAAGCCTCAATAAAGCTTG
CCTTGAGTGCTTCAAGTAGTGTGTGCCCGTCTGTTGTGTGACTCTGGTAACTAGAGATCCCTCAGACCCTTTTAGTCAGTGT
GGAAAATCTCTAGCAGGGCCCGTTTAAACCCGCTGATCAGCCTCGACTGTGCCTTCTAGTTGCCAGCCATCTGTTGTTTGCC
CCTCCCCCGTGCCTTCCTTGACCCTGGAAGGTGCCACTCCCACTGTCCTTTCCTAATAAAATGAGGAAATTGCATCGCATTG
TCTGAGTAGGTGTCATTCTATTCTGGGGGGTGGGGTGGGGCAGGACAGCAAGGGGGAGGATTGGGAAGACAATAGCAGGC
ATGCTGGGGATGCGGTGGGCTCTATGGCTTCTGAGGCGGAAAGAACCAGCTGGGGCTCTAGGGGGTATCCCCACGCGCCC
TGTAGCGGCGCATTAAGCGCGGCGGGTGTGGTGGTTACGCGCAGCGTGACCGCTACACTTGCCAGCGCCCTAGCGCCCGC
TCCTTTCGCTTTCTTCCCTTCCTTTCTCGCCACGTTCGCCGGCTTTCCCCGTCAAGCTCTAAATCGGGGGCTCCCTTTAGGGT
TCCGATTTAGTGCTTTACGGCACCTCGACCCCAAAAAACTTGATTAGGGTGATGGTTCACGTAGTGGGCCATCGCCCTGATA
GACGGTTTTTCGCCCTTTGACGTTGGAGTCCACGTTCTTTAATAGTGGACTCTTGTTCCAAACTGGAACAACACTCAACCCTA
TCTCGGTCTATTCTTTTGATTTATAAGGGATTTTGCCGATTTCGGCCTATTGGTTAAAAAATGAGCTGATTTAACAAAAATTTAA
CGCGAATTAATTCTGTGGAATGTGTGTCAGTTAGGGTGTGGAAAGTCCCCAGGCTCCCCAGCAGGCAGAAGTATGCAAAGC
ATGCATCTCAATTAGTCAGCAACCAGGTGTGGAAAGTCCCCAGGCTCCCCAGCAGGCAGAAGTATGCAAAGCATGCATCTCA
ATTAGTCAGCAACCATAGTCCCGCCCCTAACTCCGCCCATCCCGCCCCTAACTCCGCCCAGTTCCGCCCATTCTCCGCCCC
ATGGCTGACTAATTTTTTTTATTTATGCAGAGGCCGAGGCCGCCTCTGCCTCTGAGCTATTCCAGAAGTAGTGAGGAGGCTTT
TTTGGAGGCCTAGGCTTTTGCAAAAAGCTCCCGGGAGCTTGTATATCCATTTTCGGATCTGATCAGCACGTGTTGACAATTAA
TCATCGGCATAGTATATCGGCATAGTATAATACGACAAGGTGAGGAACTAAACCATGGCCAAGTTGACCAGTGCCGTTCCGG
TGCTCACCGCGCGCGACGTCGCCGGAGCGGTCGAGTTCTGGACCGACCGGCTCGGGTTCTCCCGGGACTTCGTGGAGGA
CGACTTCGCCGGTGTGGTCCGGGACGACGTGACCCTGTTCATCAGCGCGGTCCAGGACCAGGTGGTGCCGGACAACACCC
TGGCCTGGGTGTGGGTGCGCGGCCTGGACGAGCTGTACGCCGAGTGGTCGGAGGTCGTGTCCACGAACTTCCGGGACGC
CTCCGGGCCGGCCATGACCGAGATCGGCGAGCAGCCGTGGGGGCGGGAGTTCGCCCTGCGCGACCCGGCCGGCAACTG
CGTGCACTTCGTGGCCGAGGAGCAGGACTGACACGTGCTACGAGATTTCGATTCCACCGCCGCCTTCTATGAAAGGTTGGG
CTTCGGAATCGTTTTCCGGGACGCCGGCTGGATGATCCTCCAGCGCGGGGATCTCATGCTGGAGTTCTTCGCCCACCCCAA
CTTGTTTATTGCAGCTTATAATGGTTACAAATAAAGCAATAGCATCACAAATTTCACAAATAAAGCATTTTTTTCACTGCATTCT
AGTTGTGGTTTGTCCAAACTCATCAATGTATCTTATCATGTCTGTATACCGTCGACCTCTAGCTAGAGCTTGGCGTAATCATG
GTCATAGCTGTTTCCTGTGTGAAATTGTTATCCGCTCACAATTCCACACAACATACGAGCCGGAAGCATAAAGTGTAAAGCCT
GGGGTGCCTAATGAGTGAGCTAACTCACATTAATTGCGTTGCGCTCACTGCCCGCTTTCCAGTCGGGAAACCTGTCGTGCCA
GCTGCATTAATGAATCGGCCAACGCGCGGGGAGAGGCGGTTTGCGTATTGGGCGCTCTTCCGCTTCCTCGCTCACTGACTC
GCTGCGCTCGGTCGTTCGGCTGCGGCGAGCGGTATCAGCTCACTCAAAGGCGGTAATACGGTTATCCACAGAATCAGGGG
ATAACGCAGGAAAGAACATGTGAGCAAAAGGCCAGCAAAAGGCCAGGAACCGTAAAAAGGCCGCGTTGCTGGCGTTTTTCC
ATAGGCTCCGCCCCCCTGACGAGCATCACAAAAATCGACGCTCAAGTCAGAGGTGGCGAAACCCGACAGGACTATAAAGAT
ACCAGGCGTTTCCCCCTGGAAGCTCCCTCGTGCGCTCTCCTGTTCCGACCCTGCCGCTTACCGGATACCTGTCCGCCTTTC
TCCCTTCGGGAAGCGTGGCGCTTTCTCATAGCTCACGCTGTAGGTATCTCAGTTCGGTGTAGGTCGTTCGCTCCAAGCTGG
GCTGTGTGCACGAACCCCCCGTTCAGCCCGACCGCTGCGCCTTATCCGGTAACTATCGTCTTGAGTCCAACCCGGTAAGAC
ACGACTTATCGCCACTGGCAGCAGCCACTGGTAACAGGATTAGCAGAGCGAGGTATGTAGGCGGTGCTACAGAGTTCTTGA
AGTGGTGGCCTAACTACGGCTACACTAGAAGAACAGTATTTGGTATCTGCGCTCTGCTGAAGCCAGTTACCTTCGGAAAAAG
AGTTGGTAGCTCTTGATCCGGCAAACAAACCACCGCTGGTAGCGGTGGTTTTTTTGTTTGCAAGCAGCAGATTACGCGCAGA
AAAAAAGGATCTCAAGAAGATCCTTTGATCTTTTCTACGGGGTCTGACGCTCAGTGGAACGAAAACTCACGTTAAGGGATTTT
GGTCATGAGATTATCAAAAAGGATCTTCACCTAGATCCTTTTAAATTAAAAATGAAGTTTTAAATCAATCTAAAGTATATATGAG
TAAACTTGGTCTGACAGTTACCAATGCTTAATCAGTGAGGCACCTATCTCAGCGATCTGTCTATTTCGTTCATCCATAGTTGC
CTGACTCCCCGTCGTGTAGATAACTACGATACGGGAGGGCTTACCATCTGGCCCCAGTGCTGCAATGATACCGCGAGACCC
ACGCTCACCGGCTCCAGATTTATCAGCAATAAACCAGCCAGCCGGAAGGGCCGAGCGCAGAAGTGGTCCTGCAACTTTATC
CGCCTCCATCCAGTCTATTAATTGTTGCCGGGAAGCTAGAGTAAGTAGTTCGCCAGTTAATAGTTTGCGCAACGTTGTTGCCA
TTGCTACAGGCATCGTGGTGTCACGCTCGTCGTTTGGTATGGCTTCATTCAGCTCCGGTTCCCAACGATCAAGGCGAGTTAC
ATGATCCCCCATGTTGTGCAAAAAAGCGGTTAGCTCCTTCGGTCCTCCGATCGTTGTCAGAAGTAAGTTGGCCGCAGTGTTA
TCACTCATGGTTATGGCAGCACTGCATAATTCTCTTACTGTCATGCCATCCGTAAGATGCTTTTCTGTGACTGGTGAGTACTC
AACCAAGTCATTCTGAGAATAGTGTATGCGGCGACCGAGTTGCTCTTGCCCGGCGTCAATACGGGATAATACCGCGCCACAT
AGCAGAACTTTAAAAGTGCTCATCATTGGAAAACGTTCTTCGGGGCGAAAACTCTCAAGGATCTTACCGCTGTTGAGATCCA
GTTCGATGTAACCCACTCGTGCACCCAACTGATCTTCAGCATCTTTTACTTTCACCAGCGTTTCTGGGTGAGCAAAAACAGGA
AGGCAAAATGCCGCAAAAAAGGGAATAAGGGCGACACGGAAATGTTGAATACTCATACTCTTCCTTTTTCAATATTATTGAAG
CATTTATCAGGGTTATTGTCTCATGAGCGGATACATATTTGAATGTATTTAGAAAAATAAACAAATAGGGGTTCCGCGCACATT
TCCCCGAAAAGTGCCACCTGAC

### EXAMPLE 1 REFERENCES

Guralnik, J. M., et al., Prevalence of anemia in persons 65 years and older in the United States: evidence for a high rate of unexplained anemia. Blood, 2004. 104(8): p. 2263-2268.
Aul, C., N. Gattermann, and W. Schneider, Age-related incidence and other epidemiological aspects of myelodysplastic syndromes. Br J Haematol, 1992. 82(2): p. 358-67.
Montecino-Rodriguez, E., B. Berent-Maoz, and K. Dorshkind, Causes, consequences, and reversal of immune system aging. The Journal of Clinical Investigation, 2013. 123(3): p. 958-965.
Kwok, B., et al., MDS-associated somatic mutations and clonal hematopoiesis are common in idiopathic cytopenias of undetermined significance. Blood, 2015. 126(21): p. 2355-2361.
Chambers, S. M., et al., Aging hematopoietic stem cells decline in function and exhibit epigenetic dysregulation. PLoS Biol, 2007. 5(8): p. e201.
Rossi, D. J., et al., Cell intrinsic alterations underlie hematopoietic stem cell aging. Proc Natl Acad Sci USA, 2005. 102(26): p. 9194-9.
Morrison, S. J., et al., The aging of hematopoietic stem cells. Nat Med, 1996. 2(9): p. 1011-6.
Dykstra, B., et al., Clonal analysis reveals multiple functional defects of aged murine hematopoietic stem cells. J Exp Med, 2011. 208(13): p. 2691-703.
Pang, W. W., et al., Human bone marrow hematopoietic stem cells are increased in frequency and myeloid-biased with age. Proc Natl Acad Sci USA, 2011. 108(50): p. 20012-7.
Flach, J., et al., Replication stress is a potent driver of functional decline in ageing haematopoietic stem cells. Nature, 2014. 512(7513): p. 198-202.
Beerman, I., et al., Quiescent hematopoietic stem cells accumulate DNA damage during aging that is repaired upon entry into cell cycle. Cell Stem Cell, 2014. 15(1): p. 37-50.
Xie, M., et al., Age-related mutations associated with clonal hematopoietic expansion and malignancies. Nat Med, 2014.
Busque, L., et al., Recurrent somatic TET2 mutations in normal elderly individuals with clonal hematopoiesis. Nature genetics, 2012. 44(11): p. 1179-81.
Jaiswal, S., et al., Age-related clonal hematopoiesis associated with adverse outcomes. N Engl J Med, 2014. 371(26): p. 2488-98.
Genovese, G., et al., Clonal Hematopoiesis and Blood-Cancer Risk Inferred from Blood DNA Sequence. New England Journal of Medicine, 2014. 371(26): p. 2477-2487.
Adelman, E. R., et al., Aging Human Hematopoietic Stem Cells Manifest Profound Epigenetic Reprogramming of Enhancers That May Predispose to Leukemia. Cancer Discov, 2019.
Adelman, E. R., J. Shi, and M. E. Figueroa, Aging Human Hematopoietic Stem Cells Manifest Massive Epigenetic Reprogramming and Altered Gene Splicing of Key Hematopoietic Gene Sets. Blood, 2016. 128(22): p. 885.
Broske, A. M., et al., DNA methylation protects hematopoietic stem cell multipotency from myeloerythroid restriction. Nat Genet, 2009. 41(11): p. 1207-15.
Trowbridge, J. J., et al., DNA methyltransferase 1 is essential for and uniquely regulates hematopoietic stem and progenitor cells. Cell Stem Cell, 2009. 5(4): p. 442-9.
Challen, G. A., et al., Dnmt3a is essential for hematopoietic stem cell differentiation. Nat Genet, 2012. 44(1): p. 23-31.
Moran-Crusio, K., et al., Tet2 loss leads to increased hematopoietic stem cell self-renewal and myeloid transformation. Cancer Cell, 2011. 20(1): p. 11-24.
Quivoron, C., et al., TET2 inactivation results in pleiotropic hematopoietic abnormalities in mouse and is a recurrent event during human lymphomagenesis. Cancer Cell, 2011. 20(1): p. 25-38.
Ko, M., et al., Ten-Eleven-Translocation 2 (TET2) negatively regulates homeostasis and differentiation of hematopoietic stem cells in mice. Proc Natl Acad Sci USA, 2011. 108(35): p. 14566-71.
Abdel-Wahab, O., et al., Deletion of Asxl1 results in myelodysplasia and severe developmental defects in vivo. J Exp Med, 2013. 210(12): p. 2641-59.
Kim, E., et al., SRSF2 Mutations Contribute to Myelodysplasia by Mutant-Specific Effects on Exon Recognition. Cancer Cell, 2015. 27(5): p. 617-30.
Ma, X., et al., Myelodysplastic syndromes: incidence and survival in the United States. Cancer, 2007. 109(8): p. 1536-42.
Cogle, C. R., et al., Incidence of the myelodysplastic syndromes using a novel claims-based algorithm: high number of uncaptured cases by cancer registries. Blood, 2011. 117(26): p. 7121-5.
Figueroa, M. E., et al., MDS and secondary AML display unique patterns and abundance of aberrant DNA methylation. Blood, 2009. 114(16): p. 3448-58.
Figueroa, M. E., et al., DNA methylation signatures identify biologically distinct subtypes in acute myeloid leukemia. Cancer Cell, 2010. 17(1): p. 13-27.
Prebet, T., et al., Prolonged Administration of Azacitidine With or Without Entinostat for Myelodysplastic Syndrome and Acute Myeloid Leukemia With Myelodysplasia-Related Changes: Results of the US Leukemia Intergroup Trial E1905. Journal of Clinical Oncology, 2014. 32(12): p. 1242-1248.
Jiang, Y., et al., Aberrant DNA methylation is a dominant mechanism in MDS progression to AML. Blood, 2008. 113(6): p. 1315-1325.
Papaemmanuil, E., et al., Clinical and biological implications of driver mutations in myelodysplastic syndromes. Blood, 2013. 122(22): p. 3616-27; quiz 3699.
Jiang, J., et al., A core Klf circuitry regulates self-renewal of embryonic stem cells. Nat Cell Biol, 2008. 10(3): p. 353-60.
McConnell, B. B. and V. W. Yang, Mammalian Kruppel-like factors in health and diseases. Physiol Rev, 2010. 90(4): p. 1337-81.
Yamanaka, S., Strategies and new developments in the generation of patient-specific pluripotent stem cells. Cell Stem Cell, 2007. 1(1): p. 39-49.
Wu, J. and J. B. Lingrel, KLF2 inhibits Jurkat T leukemia cell growth via upregulation of cyclin-dependent kinase inhibitor p21WAF1/CIP1. Oncogene, 2004. 23(49): p. 8088-96.
Turner, J. and M. Crossley, Basic Kruppel-like factor functions within a network of interacting haematopoietic transcription factors. Int J Biochem Cell Biol, 1999. 31(10): p. 1169-74.
Lang, U. E., et al., GSK3beta phosphorylation of the KLF6 tumor suppressor promotes its transactivation of p21. Oncogene, 2013. 32(38): p. 4557-64.
Li, D., et al., Regulation of Kruppel-like factor 6 tumor suppressor activity by acetylation. Cancer Res, 2005. 65(20): p. 9216-25.
Narla, G., et al., A germline DNA polymorphism enhances alternative splicing of the KLF6 tumor suppressor gene and is associated with increased prostate cancer risk. Cancer Res, 2005. 65(4): p. 121322.
Sangodkar, J., et al., Targeted reduction of KLF6-SV1 restores chemotherapy sensitivity in resistant lung adenocarcinoma. Lung Cancer, 2009. 66(3): p. 292-7.
Tahara, E., et al., Activation of protein kinase C by phorbol 12-myristate 13-acetate suppresses the growth of lung cancer cells through KLF6 induction. Cancer Biol Ther, 2009. 8(9): p. 801-7.
Teixeira, M. S., et al., KLF6 allelic loss is associated with tumor recurrence and markedly decreased survival in head and neck squamous cell carcinoma. Int J Cancer, 2007. 121(9): p. 1976-83.
Humbert, M., et al., Deregulated expression of Kruppel-like factors in acute myeloid leukemia. Leuk Res, 2011. 35(7): p. 909-13.
Matsumoto, N., et al., Developmental regulation of yolk sac hematopoiesis by Kruppel-like factor 6. Blood, 2006. 107(4): p. 1357-65.
Tarocchi, M., et al., Carcinogen-induced hepatic tumors in KLF6+/−mice recapitulate aggressive human hepatocellular carcinoma associated with p53 pathway deregulation. Hepatology, 2011. 54(2): p. 52231.
Canver, M. C., et al., BCL11A enhancer dissection by Cas9-mediated in situ saturating mutagenesis. Nature, 2015. 527(7577): p. 192-7.
Gundry, M. C., et al., Highly Efficient Genome Editing of Murine and Human Hematopoietic Progenitor Cells by CRISPR/Cas9. Cell Rep, 2016. 17(5): p. 1453-1461.
Tothova, Z., et al., Multiplex CRISPR/Cas9-Based Genome Editing in Human Hematopoietic Stem Cells Models Clonal Hematopoiesis and Myeloid Neoplasia. Cell Stem Cell, 2017. 21(4): p. 547-555.e8.
Hilton, I. B., et al., Epigenome editing by a CRISPR-Cas9-based acetyltransferase activates genes from promoters and enhancers. Nat Biotechnol, 2015. 33(5): p. 510-7.
Kabadi, A. M., et al., Multiplex CRISPR/Cas9-based genome engineering from a single lentiviral vector. Nucleic Acids Res, 2014. 42(19): p. e147.
Coughlan, A. M., et al., Myeloid Engraftment in Humanized Mice: Impact of Granulocyte-Colony Stimulating Factor Treatment and Transgenic Mouse Strain. Stem Cells Dev, 2016. 25(7): p. 530-41.
Wunderlich, M., et al., AML xenograft efficiency is significantly improved in NOD/SCID-IL2RG mice constitutively expressing human SCF, GM-CSF and IL-3. Leukemia, 2010. 24(10): p. 1785-8.
Nicolini, F. E., et al., NOD/SCID mice engineered to express human IL-3, GM-CSF and Steel factor constitutively mobilize engrafted human progenitors and compromise human stem cell regeneration. Leukemia, 2004. 18(2): p. 341-347.
Sydor, S., et al., Kruppel-like factor 6 is a transcriptional activator of autophagy in acute liver injury. Sci Rep, 2017. 7(1): p. 8119.
Zhang, Y., et al., Model-based analysis of ChIP-Seq (MACS). Genome Biology, 2008. 9(9): p. R137.
Qunhua Li, J. B. B., Haiyan Huang, Peter J. Bickel, Measuring reproducibility of high-throughput experiments. The Annals of Applied Statistics, 2011. 5(3): p. 1752-1779.
Ross-Innes, C. S., et al., Differential oestrogen receptor binding is associated with clinical outcome in breast cancer. Nature, 2012. 481(7381): p. 389-93.
Welch, R. P., et al., ChIP-Enrich: gene set enrichment testing for ChIP-seq data. Nucleic Acids Res, 2014. 42(13): p. e105.
Beck, D., et al., Genome-wide analysis of transcriptional regulators in human HSPCs reveals a densely interconnected network of coding and noncoding genes. Blood, 2013. 122(14): p. e12-e22.
Li, D., et al., Kruppel-like factor-6 promotes preadipocyte differentiation through histone deacetylase 3-dependent repression of DLK1. J Biol Chem, 2005. 280(29): p. 26941-52.
Li, P., et al., BiolD: A Proximity-Dependent Labeling Approach in Proteomics Study. Methods Mol Biol, 2019. 1871: p. 143-151.
Jiang, N., et al., CBX4 transcriptionally suppresses KLF6 via interaction with HDAC1 to exert oncogenic activities in clear cell renal cell carcinoma. EBioMedicine, 2020. 53: p. 102692.
Chen, S. G., et al., LncRNA CASC11 promotes the development of esophageal carcinoma by regulating KLF6. Eur Rev Med Pharmacol Sci, 2019. 23(20): p. 8878-8887.
Han, X. T., et al., Circular RNA circ-ABCB10 promotes the proliferation and invasion of thyroid cancer by targeting KLF6. Eur Rev Med Pharmacol Sci, 2020. 24(3): p. 1271-1277.
Yang, H., et al., Gain of function of ASXL1 truncating protein in the pathogenesis of myeloid malignancies. Blood, 2018. 131(3): p. 328-341.
Nakahara, F., et al., Engineering a haematopoietic stem cell niche by revitalizing mesenchymal stromal cells. Nat Cell Biol, 2019. 21(5): p. 560-567.
Zalatan, J. G., et al., Engineering complex synthetic transcriptional programs with CRISPR RNA scaffolds. Cell, 2015. 160(1-2): p. 339-50.

### EXAMPLE 2 REFERENCES

1. Busque, L., et al., Recurrent somatic TET2 mutations in normal elderly individuals with clonal hematopoiesis. Nat Genet, 2012. 44(11): p. 1179-81.
2. Genovese, G., et al., Clonal hematopoiesis and blood-cancer risk inferred from blood DNA sequence. N Engl J Med, 2014. 371(26): p. 2477-87.
3. Janssens, G. E., et al., Protein biogenesis machinery is a driver of replicative aging in yeast. Elife, 2015. 4: p. e08527.
4. Xie, M., et al., Age-related mutations associated with clonal hematopoietic expansion and malignancies. Nat Med, 2014. 20(12): p. 1472-8.
5. Chambers, S. M., et al., Aging hematopoietic stem cells decline in function and exhibit epigenetic dysregulation. PLoS Biol, 2007. 5(8): p. e201.
6. Dykstra, B., et al., Clonal analysis reveals multiple functional defects of aged murine hematopoietic stem cells. J Exp Med, 2011. 208(13): p. 2691-703.
7. Morrison, S. J., et al., The aging of hematopoietic stem cells. Nat Med, 1996. 2(9): p. 1011-6.
8. Pang, W. W., et al., Human bone marrow hematopoietic stem cells are increased in frequency and myeloid-biased with age. Proc Natl Acad Sci USA, 2011. 108(50): p. 20012-7.
9. Beerman, I., et al., Quiescent hematopoietic stem cells accumulate DNA damage during aging that is repaired upon entry into cell cycle. Cell Stem Cell, 2014. 15(1): p. 37-50.
10. Flach, J., et al., Replication stress is a potent driver of functional decline in ageing haematopoietic stem cells. Nature, 2014. 512(7513): p. 198-202.
11. Mohrin, M., et al., Hematopoietic stem cell quiescence promotes error-prone DNA repair and mutagenesis. Cell Stem Cell, 2010. 7(2): p. 174-85.
12. Rossi, D. J., et al., Deficiencies in DNA damage repair limit the function of haematopoietic stem cells with age. Nature, 2007. 447(7145): p. 725-9.
13. Rube, C. E., et al., Accumulation of DNA damage in hematopoietic stem and progenitor cells during human aging. PLoS One, 2011. 6(3): p. e17487.
14. Ito, K., et al., Regulation of oxidative stress by ATM is required for self-renewal of haematopoietic stem cells. Nature, 2004. 431(7011): p. 997-1002.
15. Ito, K., et al., Reactive oxygen species act through p38 MAPK to limit the lifespan of hematopoietic stem cells. Nat Med, 2006. 12(4): p. 446-51.
16. Florian, M. C., et al., A canonical to non-canonical Wnt signalling switch in haematopoietic stem-cell ageing. Nature, 2013. 503(7476): p. 392-6.
17. Ho, T. T., et al., Autophagy maintains the metabolism and function of young and old stem cells. Nature, 2017. 543(7644): p. 205-210.
18. Papaemmanuil, E., et al., Clinical and biological implications of driver mutations in myelodysplastic syndromes. Blood, 2013. 122(22): p. 3616-27; quiz 3699.
19. Butler, J. M., H. Kobayashi, and S. Rafii, Instructive role of the vascular niche in promoting tumour growth and tissue repair by angiocrine factors. Nat Rev Cancer, 2010. 10(2): p. 138-46.
20. Ramalingam, P., et al., Chronic activation of endothelial MAPK disrupts hematopoiesis via NFKB dependent inflammatory stress reversible by SCGF. Nat Commun, 2020. 11(1): p. 666.
21. Adelman, E. R., et al., Aging Human Hematopoietic Stem Cells Manifest Profound Epigenetic Reprogramming of Enhancers That May Predispose to Leukemia. Cancer Discov, 2019. 9(8): p. 1080-1101.
22. Jiang, J., et al., A core Klf circuitry regulates self-renewal of embryonic stem cells. Nat Cell Biol, 2008. 10(3): p. 353-60.
23. McConnell, B. B. and V. W. Yang, Mammalian Kruppel-like factors in health and diseases. Physiol Rev, 2010. 90(4): p. 1337-81.
24. Takahashi, K., et al., Induction of pluripotent stem cells from adult human fibroblasts by defined factors. Cell, 2007. 131(5): p. 861-72.
25. Brey, C. W., et al., Kruppel-like family of transcription factors: an emerging new frontier in fat biology. Int J Biol Sci, 2009. 5(6): p. 622-36.
26. Matsumura, T., et al., The deacetylase HDAC1 negatively regulates the cardiovascular transcription factor Kruppel-like factor 5 through direct interaction. J Biol Chem, 2005. 280(13): p. 12123-9.
27. Miyamoto, S., et al., Positive and negative regulation of the cardiovascular transcription factor KLF5 by p300 and the oncogenic regulator SET through interaction and acetylation on the DNA-binding domain. Mol Cell Biol, 2003. 23(23): p. 8528-41.
28. Rosen, E. D. and B. M. Spiegelman, Adipocytes as regulators of energy balance and glucose homeostasis. Nature, 2006. 444(7121): p. 847-53.
29. Rosen, E. D., et al., Transcriptional regulation of adipogenesis. Genes Dev, 2000. 14(11): p. 1293-307.
30. Shindo, T., et al., Kruppel-like zinc-finger transcription factor KLF5/BTEB2 is a target for angiotensin II signaling and an essential regulator of cardiovascular remodeling. Nat Med, 2002. 8(8): p. 856-63.
31. Teixeira, M. S., et al., KLF6 allelic loss is associated with tumor recurrence and markedly decreased survival in head and neck squamous cell carcinoma. Int J Cancer, 2007. 121(9): p. 1976-83.
32. Tong, D., et al., Expression of KLF5 is a prognostic factor for disease-free survival and overall survival in patients with breast cancer. Clin Cancer Res, 2006. 12(8): p. 2442-8.
33. Wang, B., et al., The Kruppel-like factor KLF15 inhibits connective tissue growth factor (CTGF) expression in cardiac fibroblasts. J Mol Cell Cardiol, 2008. 45(2): p. 193-7.
34. Sun, D., et al., Epigenomic Profiling of Young and Aged HSCs Reveals Concerted Changes during Aging that Reinforce Self-Renewal. Cell Stem Cell, 2014. 14(5): p. 673-88.
35. Matsumoto, N., et al., Developmental regulation of yolk sac hematopoiesis by Kruppel-like factor 6. Blood, 2006. 107(4): p. 1357-65.
36. Xue, Y., et al., The Vascular Niche Regulates Hematopoietic Stem and Progenitor Cell Lodgment and Expansion via klf6a-ccl25b. Dev Cell, 2017. 42(4): p. 349-362.e4.
37. Lin, S., et al., Enhanced homology-directed human genome engineering by controlled timing of CRISPR/Cas9 delivery. Elife, 2014. 3: p. e04766.
38. Schumann, K., et al., Generation of knock-in primary human T cells using Cas9 ribonucleoproteins. Proc Natl Acad Sci USA, 2015. 112(33): p. 10437-42.
39. Notta, F., et al., Isolation of single human hematopoietic stem cells capable of long-term multilineage engraftment. Science, 2011. 333(6039): p. 218-21.
40. Beck, D., et al., Genome-wide analysis of transcriptional regulators in human HSPCs reveals a densely interconnected network of coding and noncoding genes. Blood, 2013. 122(14): p. e12-22.
41. Kurita, R., et al., Establishment of immortalized human erythroid progenitor cell lines able to produce enucleated red blood cells. PLoS One, 2013. 8(3): p. e59890.
42. Kabadi, A. M., et al., Multiplex CRISPR/Cas9-based genome engineering from a single lentiviral vector. Nucleic Acids Res, 2014. 42(19): p. e147.
43. Buchwalter, A. and M. W. Hetzer, Nucleolar expansion and elevated protein translation in premature aging. Nat Commun, 2017. 8(1): p. 328.
44. Larson, K., et al., Heterochromatin formation promotes longevity and represses ribosomal RNA synthesis. PLoS Genet, 2012. 8(1): p. e1002473.
45. Montecino-Rodriguez, E., B. Berent-Maoz, and K. Dorshkind, Causes, consequences, and reversal of immune system aging. J Clin Invest, 2013. 123(3): p. 958-65.
46. Konermann, S., et al., Genome-scale transcriptional activation by an engineered CRISPR-Cas9 complex. Nature, 2015. 517(7536): p. 583-8.
47. Abad, M., et al., Reprogramming in vivo produces teratomas and iPS cells with totipotency features. Nature, 2013. 502(7471): p. 340-5.
48. Ocampo, A., et al., In Vivo Amelioration of Age-Associated Hallmarks by Partial Reprogramming. Cell, 2016. 167(7): p. 1719-1733.e12.
49. Ohnishi, K., et al., Premature termination of reprogramming in vivo leads to cancer development through altered epigenetic regulation. Cell, 2014. 156(4): p. 663-77.
50. Brown, K., et al., SI RT3 reverses aging-associated degeneration. Cell Rep, 2013. 3(2): p. 319-27.
51. Mohrin, M., et al., Stem cell aging. A mitochondrial UPR-mediated metabolic checkpoint regulates hematopoietic stem cell aging. Science, 2015. 347(6228): p. 1374-7.
52. Moiseeva, O., et al., Mitochondrial dysfunction contributes to oncogene-induced senescence. Mol Cell Biol, 2009. 29(16): p. 4495-507.
53. Passos, J. F., G. Saretzki, and T. von Zglinicki, DNA damage in telomeres and mitochondria during cellular senescence: is there a connection? Nucleic Acids Res, 2007. 35(22): p. 7505-13.
54. Mallipattu, S. K., et al., Kruppel-like factor 6 regulates mitochondrial function in the kidney. J Clin Invest, 2015. 125(3): p. 1347-61.
55. Balaban, R. S., S. Nemoto, and T. Finkel, Mitochondria, oxidants, and aging. Cell, 2005. 120(4): p. 483-95.
56. Pietras, E. M., M. R. Warr, and E. Passegué, Cell cycle regulation in hematopoietic stem cells. J Cell Biol, 2011. 195(5): p. 709-20.
57. Partridge, L., Intervening in ageing to prevent the diseases of ageing. Trends Endocrinol Metab, 2014. 25(11): p. 555-7.
58. Bryder, D., D. J. Rossi, and I. L. Weissman, Hematopoietic stem cells: the paradigmatic tissue-specific stem cell. Am J Pathol, 2006. 169(2): p. 338-46.
59. Benayoun, B. A., E. A. Pollina, and A. Brunet, Epigenetic regulation of ageing: linking environmental inputs to genomic stability. Nat Rev Mol Cell Biol, 2015. 16(10): p. 593-610.
60. Sen, P., et al., Epigenetic Mechanisms of Longevity and Aging. Cell, 2016. 166(4): p. 822-839.
61. Liu, G. H., et al., Recapitulation of premature ageing with iPSCs from Hutchinson-Gilford progeria syndrome. Nature, 2011. 472(7342): p. 221-5.
62. Polo, J. M., et al., A molecular roadmap of reprogramming somatic cells into iPS cells. Cell, 2012. 151(7): p. 1617-32.
63. Lapasset, L., et al., Rejuvenating senescent and centenarian human cells by reprogramming through the pluripotent state. Genes Dev, 2011. 25(21): p. 2248-53.
64. Mahmoudi, S. and A. Brunet, Aging and reprogramming: a two-way street. Curr Opin Cell Biol, 2012. 24(6): p. 744-56.
65. Rando, T. A. and H. Y. Chang, Aging, rejuvenation, and epigenetic reprogramming: resetting the aging clock. Cell, 2012. 148(1-2): p. 46-57.
66. Zou, Z., et al., A Single-Cell Transcriptomic Atlas of Human Skin Aging. Dev Cell, 2021. 56(3): p. 383-397.e8.
67. Lu, Y., et al., Reprogramming to recover youthful epigenetic information and restore vision. Nature, 2020. 588(7836): p. 124-129.
68. Sarkar, T. J., et al., Transient non-integrative expression of nuclear reprogramming factors promotes multifaceted amelioration of aging in human cells. Nat Commun, 2020. 11(1): p. 1545.
69. Gratwohl, A., et al., Hematopoietic stem cell transplantation: a global perspective. Jama, 2010. 303(16): p. 1617-24.
70. Niederwieser, D., et al., Hematopoietic stem cell transplantation activity worldwide in 2012 and a SWOT analysis of the Worldwide Network for Blood and Marrow Transplantation Group including the global survey. Bone Marrow Transplant, 2016. 51(6): p. 778-85.
71. Gratwohl, A., H. Baldomero, and J. Passweg, Hematopoietic stem cell transplantation activity in Europe. Curr Opin Hematol, 2013. 20(6): p. 485-93.
72. Finke, J., et al., Prognostic factors affecting outcome after allogeneic transplantation for hematological malignancies from unrelated donors: results from a randomized trial. Biol Blood Marrow Transplant, 2012. 18(11): p. 1716-26.
73. Kollman, C., et al., The effect of donor characteristics on survival after unrelated donor transplantation for hematologic malignancy. Blood, 2016. 127(2): p. 260-7.
74. Bak, R. O., D. P. Dever, and M. H. Porteus, CRISPR/Cas9 genome editing in human hematopoietic stem cells. Nat Protoc, 2018. 13(2): p. 358-376.
75. Danet, G. H., et al., Expansion of human SCID-repopulating cells under hypoxic conditions. J Clin Invest, 2003. 112(1): p. 126-35.
76. Koller, M. R., et al., Expansion of primitive human hematopoietic progenitors in a perfusion bioreactor system with IL-3, IL-6, and stem cell factor. Biotechnology (N Y), 1993. 11(3): p. 358-63.
77. Vinjamur, D. S. and D. E. Bauer, Growing and Genetically Manipulating Human Umbilical Cord Blood-Derived Erythroid Progenitor (HUDEP) Cell Lines. Methods Mol Biol, 2018. 1698: p. 275-284.
78. Moreno-Mateos, M. A., et al., CRISPRscan: designing highly efficient sgRNAs for CRISPR-Cas9 targeting in vivo. Nat Methods, 2015. 12(10): p. 982-8.
79. Gundry, M. C., et al., Highly Efficient Genome Editing of Murine and Human Hematopoietic Progenitor Cells by CRISPR/Cas9. Cell Rep, 2016. 17(5): p. 1453-1461.
80. Ran, F. A., et al., Genome engineering using the CRISPR-Cas9 system. Nat Protoc, 2013. 8(11): p. 2281-2308.
81. Korshunova, I., et al., Genetic modification increases the survival and the neuroregenerative properties of transplanted neural stem cells. JCI Insight, 2020. 5(4).
82. Martin, M., Cutadapt removes adapter sequences from high-throughput sequencing reads. EMBnet.journal; Vol 17, No 1: Next Generation Sequencing Data Analysis, 2011.
83. Dobin, A., et al., STAR: ultrafast universal RNA-seq aligner. Bioinformatics, 2013. 29(1): p. 15-21.
84. Hartley, S. W. and J. C. Mullikin, QoRTs: a comprehensive toolset for quality control and data processing of RNA-Seq experiments. BMC Bioinformatics, 2015. 16(1): p. 224.
85. Anders, S. and W. Huber, Differential expression analysis for sequence count data. Genome Biology, 2010. 11(10): p. R106.
86. Stephens, M., False discovery rates: a new deal. Biostatistics, 2017. 18(2): p. 275-294.
87. Subramanian, A., et al., Gene set enrichment analysis: a knowledge-based approach for interpreting genome-wide expression profiles. Proc Natl Acad Sci USA, 2005. 102(43): p. 15545-50.
88. Andrews, S. FastQC: a quality control tool for high throughput sequence data. 2010; Available from:
www.bioinformatics.babraham.ac.uk/projects/fastqc.
89. Martin, M., Cutadapt removes adapter sequences from high-throughput sequencing reads. 2011, 2011. 17(1).
90. Langmead, B., et al., Ultrafast and memory-efficient alignment of short DNA sequences to the human genome. Genome Biology, 2009. 10(3): p. R25.
91. Li, H., et al., The Sequence Alignment/Map format and SAMtools. Bioinformatics, 2009. 25(16): p. 2078-9.
92. Zhang, Y., et al., Model-based analysis of ChIP-Seq (MACS). Genome Biol, 2008. 9(9): p. R137.
93. Stark, R. and G. Brown, DiffBind differential binding analysis of ChIP-Seq peak data. In R package version, 2011. 100.
94. Welch, R. P., et al., ChIP-Enrich: gene set enrichment testing for ChIP-seq data. Nucleic Acids Res, 2014. 42(13): p. e105.
95. Akalin, A., et al., Genomation: a toolkit to summarize, annotate and visualize genomic intervals. Bioinformatics, 2015. 31(7): p. 1127-9.
96. Ramirez, F., et al., deepTools2: a next generation web server for deep-sequencing data analysis. Nucleic Acids Research, 2016. 44(W1): p. W160-W165.
97. Zhou, R., et al., A role for mitochondria in NLRP3 inflammasome activation. Nature, 2011. 469(7329): p. 221-5.

What is claimed is:

1. A method of preparing HSPC for bone marrow transplantation, the method comprising (a) obtaining donor hematopoietic stem and progenitor cells and (b) upregulating expression of Kruppel-like factor 6.

2. The method of claim 1, further comprising (c) administering the HSPC of step (b) to a subject in need thereof.

3. The method of claim 1, wherein step (b) comprises contacting the HSPC with CRISPR/dCas9 fused to an activation domain.

4. The method of claim 2, wherein step (b) comprises contacting the HSPC with CRISPR/dCas9 fused to an activation domain.
